(19) United States
(12) Patent Application Publication
(10) Pub. No.: US 2023/0146829 A1
(43) Pub. Date: May 11, 2023

(54) SELECTIVE ANDROGEN RECEPTOR DEGRADER (SARD) LIGANDS AND METHODS OF USE THEREOF

(71) Applicant: University of Tennessee Research Foundation, Knoxville, TN (US)

(72) Inventors: Ramesh Narayanan, Cordova, TN (US); Thamarai Ponnusamy, Mamphis, TN (US); Duane D. Miller, Collierville, TN (US); Yali He, Germantown, TN (US); Dong-Jin Hwang, Arlington, TN (US)

(21) Appl. No.: 17/894,482

(22) Filed: Aug. 24, 2022

Related U.S. Application Data
(63) Continuation of application No. PCT/US2021/019477, filed on Feb. 24, 2021.
(60) Provisional application No. 62/981,520, filed on Feb. 25, 2020.

(51) Int. Cl. A61K 31/404; A61K 31/4245; A61K 31/416; A61K 31/4439; A61K 31/422; A61K 31/4188; A61K 31/415; A61K 31/4192

(52) U.S. Cl. CPC A61K 31/404; A61K 31/4245; A61K 31/416; A61K 31/4439; A61K 31/422; A61K 31/4188; A61K 31/415; A61K 31/4192

(57) ABSTRACT

This invention is directed to selective androgen receptor degrader (SARD) compounds including heterocyclic rings and pharmaceutical compositions and uses thereof in treating prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor-full length (AR-FL) including pathogenic or resistance mutations, AR-splice variants (AR-SV), and pathogenic polyglutamine (polyQ) polymorphisms of AR in a subject.

### CROSS REFERENCE TO RELATED APPLICATIONS

This application is a continuation of PCT Application No. PCT/US2021/019477, filed Feb. 24, 2021, and claims the benefit of U.S. Provisional Application No. 62/981520, filed Feb. 25, 2020, which are incorporated in their entirety herein by reference.

### GOVERNMENT INTEREST STATEMENT

This invention was made with government support under RO1 CA229164, awarded by the National Cancer Institute. The government has certain rights in the invention.

### FIELD OF THE INVENTION

This invention is directed to selective androgen receptor degrader (SARD) compounds including heterocyclic rings and pharmaceutical compositions and uses thereof in treating prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor—full length (AR-FL) including pathogenic or resistance mutations, AR-splice variants (AR-SV), and pathogenic polyglutamine (polyQ) polymorphisms of AR in a subject.

### BACKGROUND OF THE INVENTION

Prostate cancer (PCa) is one of the most frequently diagnosed noncutaneous cancers among men in the US and is the second most common cause of cancer deaths with more than 200,000 new cases and over 30,000 deaths each year in the United States. PCa therapeutics market is growing at an annual rate of 15-20% globally.

Androgen-deprivation therapy (ADT) is the standard of treatment for advanced PCa. Patients with advanced prostate cancer undergo ADT, either by luteinizing hormone releasing hormone (LHRH) agonists, LHRH antagonists or by bilateral orchiectomy. Despite initial response to ADT, disease progression is inevitable and the cancer emerges as castration-resistant prostate cancer (CRPC). Up to 30% of patients with prostate cancer that undergo primary treatment by radiation or surgery will develop metastatic disease within 10 years of the primary treatment. Approximately 50,000 patients a year will develop metastatic disease, which is termed metastatic CRPC (mCRPC).

Patients with CRPC have a median survival of 12-18 months. Though castration-resistant, CRPC is still dependent on the androgen receptor (AR) signaling axis for continued growth. The primary reason for CRPC re-emergence is re-activation of AR by alternate mechanisms such as: 1) intracrine androgen synthesis, 2) AR splice variants (AR-SV), e.g., that lack ligand binding domain (LBD), 3) AR-LBD mutations with potential to resist AR antagonists (i.e., mutants that are not sensitive to inhibition by AR antagonists, and in some cases AR antagonists act as agonists of the AR bearing these LBD mutations), and 4) amplifications of the AR gene within the tumor. A critical barrier to progress in treating CRPC is that AR signaling inhibitors such as darolutamide, enzalutamide, apalutamide, bicalutamide, and abiraterone, acting through the LBD, fail to inhibit growth driven by the N-terminal domain (NTD)-dependent constitutively active AR-SV such as AR-V7, the most prominent AR-SV. Recent high-impact clinical trials with enzalutamide and abiraterone in CRPC patients demonstrated that just 13.9% of AR-V7-positive patients among 202 patients starting treatment with enzalutamide (Xtandi) or abiraterone acetate (Zytiga) had PSA responses to either of the treatments (Antonarakis ES, et al. J. Clin. Oncol. 2017 Apr. 6. doi: 10.1200/JCOC.2016.70.1961), indicating the requirement for next generation AR antagonists that target AR-SVs. In addition, a significant number of CRPC patients are becoming refractory to abiraterone or enzalutamide and apalutamide, and darolutamide, emphasizing the need for next generation AR antagonists.

Current evidences demonstrate that CRPC growth is dependent on constitutively active AR including AR-SV's that lack the LBD such as AR-V7 and therefore cannot be inhibited by conventional antagonists. AR inhibition and degradation through binding to a domain that is distinct from the AR LBD provides alternate strategies to manage CRPC.

Molecules that degrade the AR prevent any inadvertent AR activation through growth factors or signaling pathways, or promiscuous ligand-dependent activation. In addition, molecules that inhibit the constitutive activation of AR-SVs are extremely important to provide extended benefit to CRPC patients.

Currently only a few chemotypes are known to degrade AR which include the SARDs ARN-509, AZD-3514, and ASC-J9. However, these molecules degrade AR indirectly at much higher concentrations than their binding coefficient and they fail to degrade the AR-SVs that have become in recent years the primary reason for resurgence of treatment-resistant CRPC.

This invention describes novel AR antagonists with unique pharmacology that strongly (high potency and full efficacy) and selectively bind AR (better than known antagonists in some cases; bind to LBD and/or NTD), antagonize AR, and degrade AR full length (AR-FL) and AR-SV. Selective androgen receptor degrader (SARD) compounds possess dual degradation and AR-SV inhibitory functions and hence are distinct from any available CRPC therapeutics. These novel selective androgen receptor degrader (SARD) compounds inhibit the growth of PCa cells and tumors that are dependent on AR-FL and AR-SV for proliferation.

SARDs have the potential to evolve as new therapeutics to treat CRPCs that are untreatable with any other antagonists. This unique property of degrading AR-SV has extremely important health consequences for prostate cancer. Till date only one series of synthetic molecules (EPI-001, EPI-506, etc.) and some marine natural products such as the sinkotamides and glycerol ether Naphetenone B, are reported to bind to AR-NTD and inhibit AR function and PCa cell growth, albeit at lower affinity and inability to degrade the receptor. The SARDs reported herein also bind to AR-NTD and inhibit NTD-driven (e.g., ligand independent) AR activity.

The positive correlation between AR and PCa and the lack of an infallible AR antagonist, emphasizes the need for molecules that inhibit AR function through novel or alternate mechanisms and/or binding sites, and that can elicit antagonistic activities within an altered cellular environment.

Although traditional antiandrogens such as darolutamide, enzalutamide, apalutamide, bicalutamide and flutamide and androgen deprivation therapies (ADT) were approved for use in prostate cancer, there is significant evidence that antiandrogens could also be used in a variety of other hormone dependent and hormone independent cancers. For example, antiandrogens have been tested in breast cancer (enzalutamide; Breast Cancer Res. (2014) 16(1): R7; darolutamide in ClinicalTrials.gov Identifier: NCT03004534), non-small cell lung cancer (shRNAi AR), renal cell carcinoma (ASC-J9), partial androgen insensitivity syndrome (PAIS) associated malignancies such as gonadal tumors and seminoma, advanced pancreatic cancer (World J. Gastroenterology 20(29), 9229), cancer of the ovary, fallopian tubes, or peritoneum, cancer of the salivary gland (Head and Neck (2016) 38, 724-731; ADT was tested in AR-expressing recurrent/metastatic salivary gland cancers and was confirmed to have benefit on progression free survival and overall survival endpoints), bladder cancer (Oncotarget 6(30), 29860-29876); Int J. Endocrinol (2015), Article ID 384860), pancreatic cancer, lymphoma (including mantle cell), and hepatocellular carcinoma. Use of a more potent antiandrogen such as a SARD in these cancers may more efficaciously treat the progression of these and other cancers. Other AR-expressing cancers may also benefit from SARD treatment such as breast cancer (e.g., triple negative breast cancer (TNBC)), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer.

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, we have evidence that certain TNBC's may be supported by androgen independent activation of AR-SVs lacking the LBD or androgen-dependent activation of AR full length. As such, darolutamide, enzalutamide, apalutamide, and other LBD-directed traditional AR antagonists would not be able to antagonize AR-SVs in these TNBC's. However, SARDs of this invention which are capable of destroying AR-SVs (see Table 1 and Example 2) through a binding site in the NTD of AR (see Example 9 of US2017-0368003) would be able to antagonize AR including AR-SV observed in TNBC patient derived xenograpfts and provide an anti-tumor effect, as shown in Example 8 of US2017-0368003.

Traditional antiandrogens such as bicalutamide and flutamide were approved for use in prostate cancer. Subsequent studies have demonstrated the utility of antiandrogens (e.g., flutamide, spironolactone, cyproterone acetate, finasteride and chlormadinone acetate) in androgen-dependent dermatological conditions such as androgenic alopecia (male pattern baldness), acne vulgaris, and hirsutism (e.g., in female facial hair). Prepubertal castration prevents sebum production and androgenic alopecia but this can be reversed by use of testosterone, suggesting its androgen-dependence.

The AR gene has a polymorphism of glutamine repeats (polyQ) within exon 1 which when shortened may augment AR transactivation (i.e., hyperandrogenism). It has been found that shortened polyQ polymorphisms are more common in people with alopecia, hirsutism, and acne. Classic antiandrogens are undesirable for these purposes because they are ineffective through dermal dosing and their long-term systemic use raises the risks of untoward sexual effects such as gynecomastia and impotence. Further, similar to CPRC discussed above, inhibition of ligand-dependent AR activity alone may not be sufficient as AR can be activated by various cellular factors other than the endogeneous androgens testosterone (T) and dihydrotestosterone (DHT), such as growth factors, kinases, co-activator overexpression and/or promiscuous activation by other hormones (e.g., estrogens or glucocorticoids). Consequently, blocking the binding of T and DHT to AR with a classical antiandrogen may not be sufficient to have the desired efficacy.

An emerging concept is the topical application of a SARD to destroy the AR locally to the affected areas of the skin or other tissue without exerting any systemic antiandrogenism. For this use, a SARD that does not penetrate the skin or is rapidly metabolized would be preferrable.

Supporting this approach is the observation that cutaneous wound healing has been demonstrated to be suppressed by androgens. Castration of mice accelerates cutaneous wound healing while attenuating the inflammation in the wounds. The negative correlation between androgen levels and cutaneous healing and inflammation, in part, explains another mechanism by which high levels of endogenous androgens exacerbate hyperandrogenic dermatological conditions. Further, it provides a rationale for the treatment of wounds such as diabetic ulcers or even trauma, or skin disorders with an inflammatory component such as acne or psoriasis, with a topical SARD.

Androgenic alopecia occurs in ˜50% of Caucasian males by midlife and up to 90% by 80 years old. Minoxidil (a topical vasodilator) and finasteride (a systemic 5alpha reductase type II inhibitor) are FDA approved for alopecia but require 4-12 months of treatment to produce a therapeutic effect and only arrest hair loss in most with mild to moderate hair regrowth in 30-60%. Since currently available treatments have slow and limited efficacy that varies widely between individuals, and produce unwanted sexual side effects, it is important to find a novel approach to treat androgenic alopecia and other hyperandrogenic dermatologic diseases.

Amyotrophic lateral sclerosis (ALS) is a fatal neurodegenerative disease characterized by selective loss of upper and lower motor neurons and skeletal muscle atrophy. Epidemiologic and experimental evidence suggest the involvement of androgens in ALS pathogenesis (“Anabolic/androgenic steroid nandrolone exacerbates gene expression modifications induced by mutant SOD1 in muscles of mice models of amyotrophic lateral sclerosis.” Galbiati M, et al. Pharmacol. Res. 2012, 65(2), 221-230), but the mechanism through which androgens modify the ALS phenotype is unknown. A transgenic animal model of ALS demonstrated improved survival upon surgical castration (i.e., androgen ablation). Treatment of these castrated animals with the androgen agonist nandrolone decanoate worsened disease manifestations. Castration reduces the AR level, which may be the reason for extended survival. The survival benefit is reversed by androgen agonist (“Androgens affect muscle, motor neuron, and survival in a mouse model of SOD1-related amyotrophic lateral sclerosis.” Aggarwal T, et al. Neurobiol. Aging. 2014 35(8), 1929-1938). Notably, stimulation with nandrolone decanoate promoted the recruitment of endogenous androgen receptor into biochemical complexes that were insoluble in sodium dodecyl sulfate, a finding consistent with protein aggregation. Overall, these results shed light on the role of androgens as modifiers of ALS pathogenesis via dysregulation of androgen receptor homeostasis. Antiandrogens should block the effects of nandrolone undecanoate or endogeneous androgens and reverse the toxicities due to AR aggregation. Further, an antiandrogen that can block action of LBD-dependent AR agonists and concomitantly lower AR protein levels, such as the SARDs of this invention, would be therapeutic in ALS. Riluzole is an available drug for ALS treatment, however, it only provides short-term effects. There is an urgent need for drugs that extend the survival of ALS patients.

Androgen receptor action promotes uterine proliferation. Hyperandrogenicity of the short polyQ AR has been associated with increased leiomyoma or uterine fibroids. (Hsieh Y Y, et al. J. Assist. Reprod. Genet. 2004, 21(12), 453-457). A separate study of Brazilian women found that shorter and longer [CAG](n) repeat alleles of AR were exclusive to the leiomyoma group in their study (Rosa F E, et al. Clin. Chem. Lab. Med. 2008, 46(6), 814-823). Similarly, in Asian Indian women long polyQ AR was associated with endometriosis and leiomyoma and can be regarded as high-risk markers. SARDs could be used in women with uterine fibroids, especially those expressing shorter and longer [CAG](n) repeat alleles, to treat existing uterine fibroids, prevent worsening of fibroids and/or ameliorate carcinogenicity associated with fibroids.

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, et al. J Vasc Surg (2016) 63(6):1602-1612) showed that flutamide (50 mg/kg) or ketoconazole (150 mg/kg) attenuated porcine pancreatic elastase (0.35 U/mL) induced AAA by 84.2% and 91.5% compared to vehicle (121%). Further AR −/− mice showed attenuated AAA growth (64.4%) compared to wildtype (both treated with elastase). Correspondingly, administration of a SARD to a patient suffering from an AAA may help reverse, treat or delay progression of AAA to the point where surgery is needed.

X-linked spinal-bulbar muscular atrophy (SBMA-also known as Kennedy's disease) is a muscular atrophy that arises from a defect in the androgen receptor gene on the X chromosome. Proximal limb and bulbar muscle weakness results in physical limitations including dependence on a wheelchair in some cases. The mutation results in a protracted polyglutamine tract added to the N-terminal domain of the androgen receptor (polyQ AR). Binding and activation of this lengthened polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci.201658(3), 343-347). These steps are required for pathogenesis and result in partial loss of transactivation function (i.e., an androgen insensitivity) and a poorly understood neuromuscular degeneration. Support of the use of antiandrogens comes in a report in which the antiandrogen flutamide protects male mice from androgen-dependent toxicity in three models of spinal bulbar muscular atrophy (Renier K J, et al. Endocrinology 2014, 155(7), 2624-2634). Currently there are no disease-modifying treatments, but rather only symptom directed treatments. Efforts to target the polyQ AR of Kennedy's disease as the proximal mediator of toxicity by harnessing cellular machinery to promote its degradation, i.e., through the use of a SARD, hold promise for therapeutic intervention. Selective androgen receptor degraders such as those reported herein bind to and degrade all androgen receptors tested (full length, splice variant, antiandrogen resistance mutants, etc.) so degradation of polyQ AR polymorphism is also expected, indicating that they are promising leads for treatment of SBMA.

Here selective androgen receptor degrader (SARD) compounds are described that may bind to the LBD and/or an alternate binding and degradation domain (BDD) located in the NTD, antagonize AR, and degrade AR thereby blocking ligand-dependent and ligand-independent AR activities. This novel mechanism produces improved efficacy when dosed systemically (e.g., for prostate cancer) or topically (e.g., dermatological diseases).

### SUMMARY OF THE INVENTION

One embodiment of the invention encompasses a selective androgen receptor degrader (SARD) compound, or its isomer, optical isomer, or any mixture of optical isomers, pharmaceutically acceptable salt, pharmaceutical product, polymorph, hydrate or any combination thereof, wherein said SARD compound is represented by a compound of the following structures:

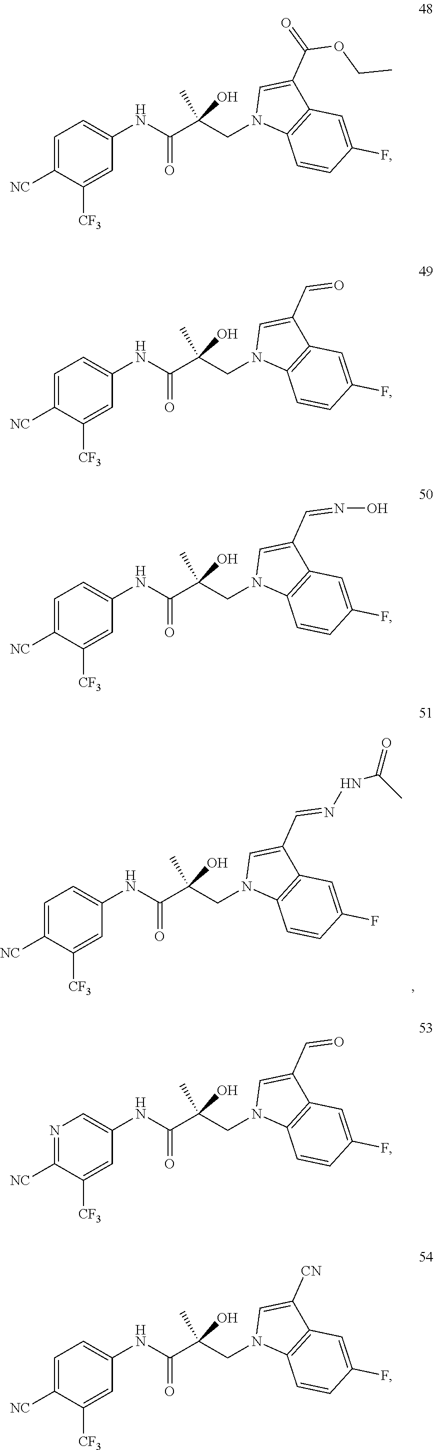 [SMILES: CCOC(=O)c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1ccc(C#N)c(C(F)(F)F)c1 ; C[C@](O)(Cn1cc(/C=N\O)c2cc(F)ccc21)C(=O)Nc1ccc(C#N)c(C(F)(F)F)c1 ; CC(=O)N/N=C/c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1cnc(C#N)c(C(F)(F)F)c1 ; C[C@](O)(Cn1cc(C#N)c2cc(F)ccc21)C(=O)Nc1ccc(C#N)c(C(F)(F)F)c1]

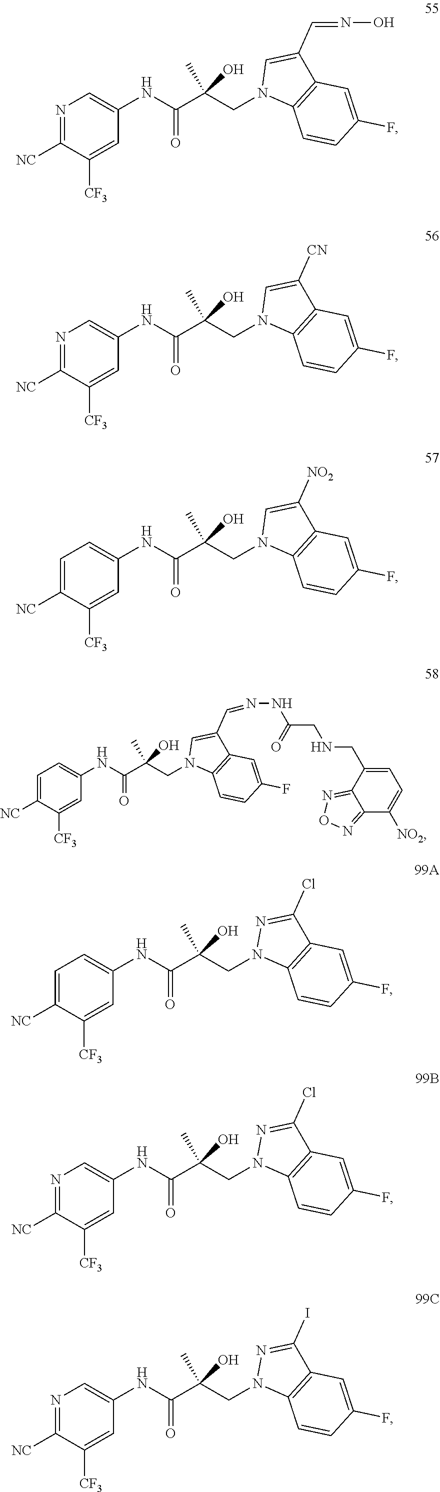

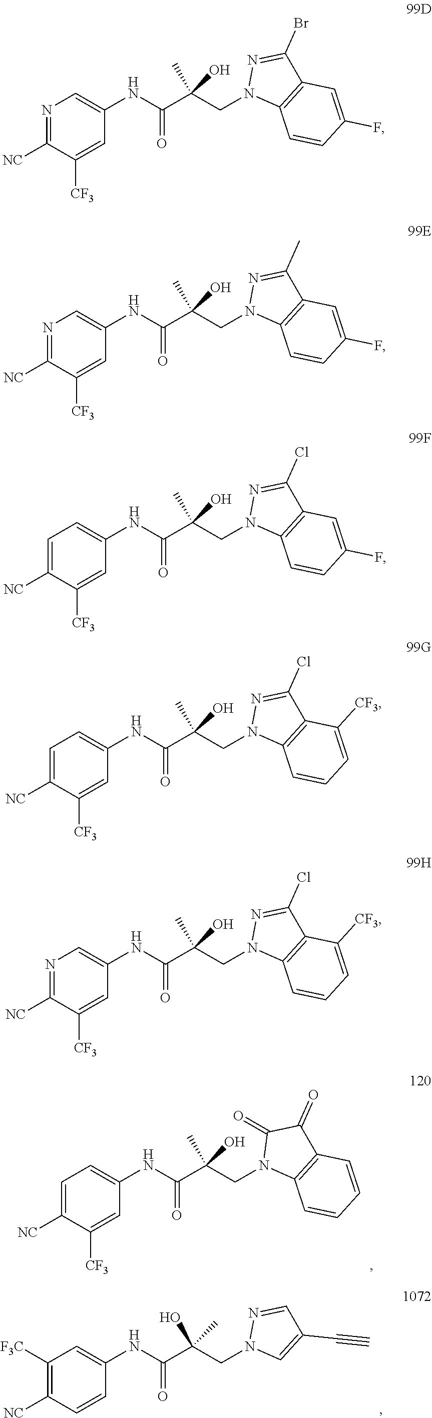

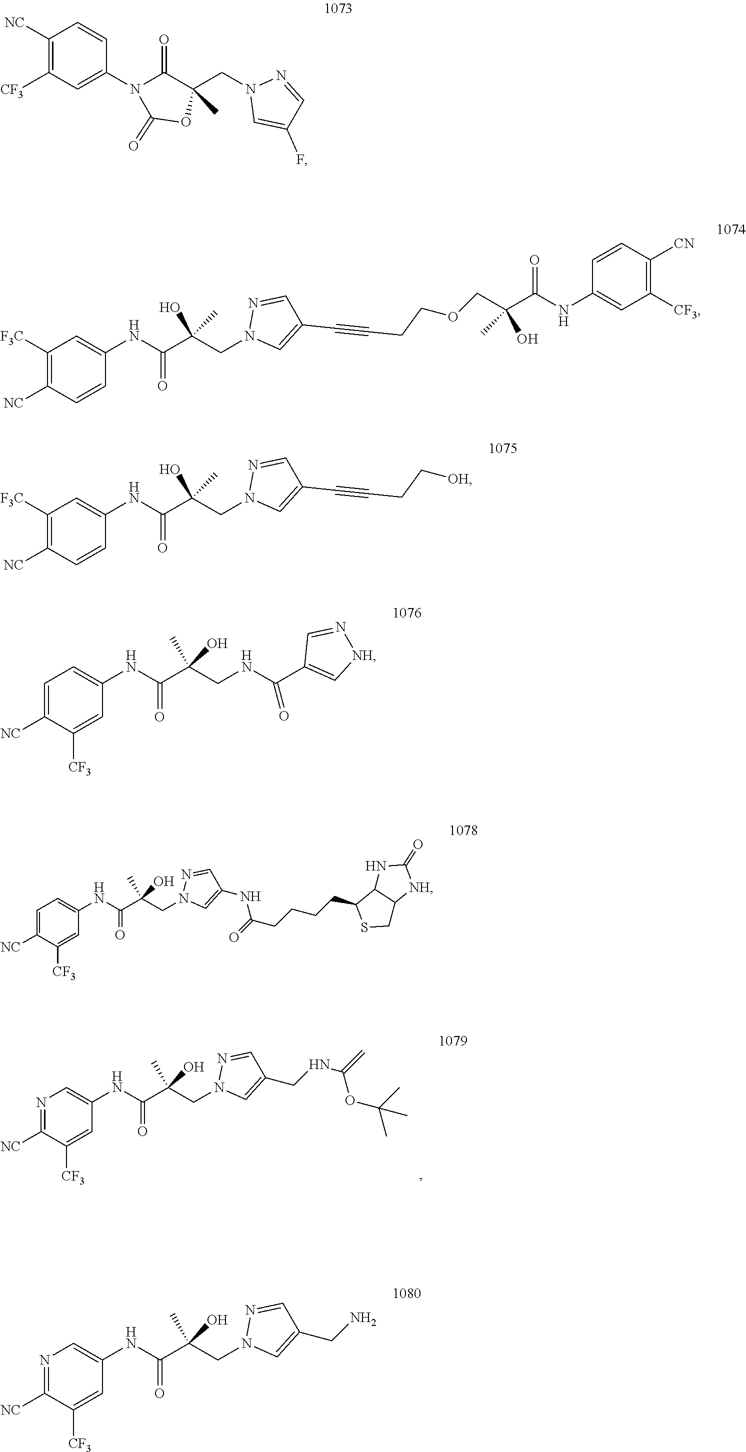

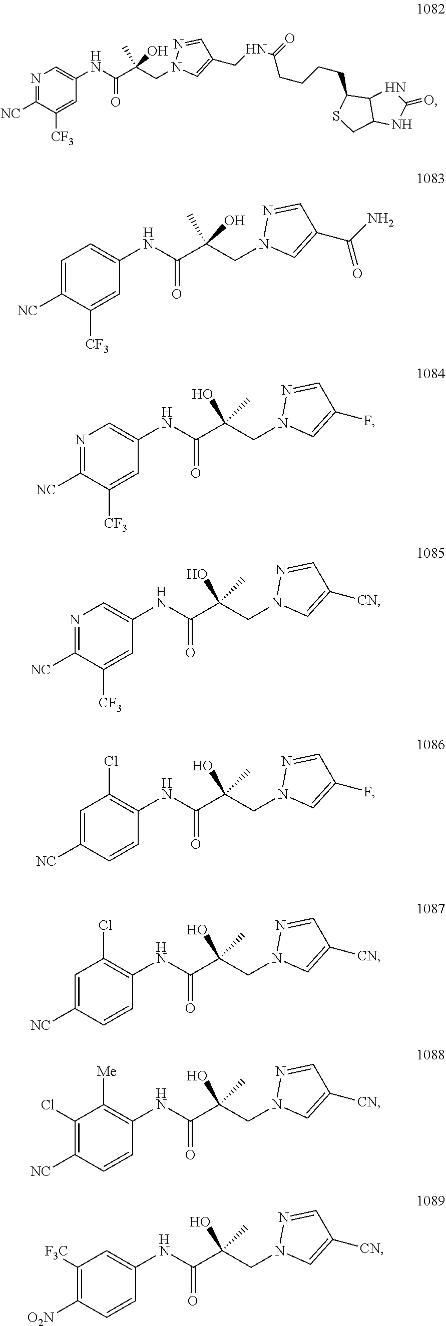

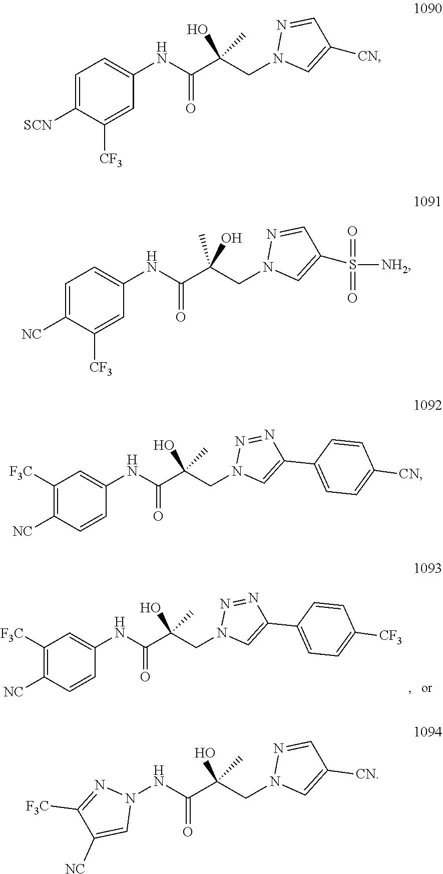

One embodiment of the invention encompasses the SARD compound having at least one of the following properties: binding to the AR through an alternate binding domain in the NTD; binds to the AR through the AR ligand binding domain (LBD); exhibits AR-splice variant (AR-SV) degradation activity; exhibits AR-full length (AR-FL) degradation activity including pathogenic mutations thereof; exhibits AR-SV inhibitory activity (i.e., is an AR-SV antagonist); exhibits AR-FL inhibitory activity (i.e., is an AR-FL antagonist) including pathogenic mutations thereof; possesses dual AR-SV degradation and AR-SV inhibitory functions; dual AR-FL degradation and AR-FL inhibitory functions and/or AR antagonism in vivo of an AR target organ.

Another embodiment of the invention encompasses pharmaceutical compositions comprising a SARD compound according to this invention, or its isomer, optical isomer, or any mixture of optical isomers, pharmaceutically acceptable salt, pharmaceutical product, polymorph, hydrate or any combination thereof, and a pharmaceutically acceptable carrier. The pharmaceutical composition may be formulated for topical use. The topical pharmaceutical composition may be a solution, lotion, salve, cream, ointment, liposome, spray, gel, foam, roller stick, cleansing soap or bar, emulsion, mousse, aerosol, or shampoo. The pharmaceutical composition may be formulated for oral use.

In another aspect, the invention provides a method of treating an androgen receptor dependent disease or condition or an androgen dependent disease or condition in a subject in need thereof, comprising administering to the subject a therapeutically effective amount of a compound of the invention as described herein. In one embodiment, an “androgen receptor dependent disease or condition” is a medical condition that is, in part or in full, dependent on, or is sensitive to, the presence of androgenic activity or activation of the AR-axis in the body. In one embodiment, an androgen dependent disease or condition is used interchangeably with an androgen receptor dependent disease or condition.

In one embodiment, the androgen receptor dependent disease or condition responds to at least one of AR-splice variant (AR-SV) degradation activity, full length (AR-FL) degradation activity, AR-SV inhibitory, or AR-FL inhibitory activity.

The invention encompasses a method of treating prostate cancer (PCa) or increasing survival in a male subject in need of treatment comprising administering to the subject a therapeutically effective amount of a compound defined by formulas 48-51, 53-58, 99A-99H, 120, 1072-1076, 1078-1080, and 1082-1094. The prostate cancer includes, but is not limited to, advanced prostate cancer, castration resistant prostate cancer (CRPC), metastatic CRPC (mCRPC), non-metastatic CRPC (nmCRPC), high-risk nmCRPC or any combination thereof. Another embodiment of the invention encompasses the method further comprising administering androgen deprivation therapy (ADT). Alternatively, the method may treat a prostate or other cancer that is resistant to treatment with known androgen receptor antagonist(s) or ADT. In another embodiment, the method may treat enzalutamide resistant prostate cancer. In another embodiment, the method may treat apalutamide resistant prostate cancer. In another embodiment, the method may treat abiraterone resistant prostate cancer. In another embodiment, the method may treat darolutamide resistant prostate cancer. Yet another embodiment of the invention encompasses a method of treating prostate or other AR antagonist resistant cancer with a SARD compound of the invention wherein the androgen receptor antagonist(s) is at least one of darolutamide, apalutamide, enzalutamide, bicalutamide, abiraterone, EPI-001, EPI-506, AZD-3514, galeterone, ASC-J9, flutamide, hydroxyflutamide, nilutamide, cyproterone acetate, ketoconazole, or spironolactone.

In some embodiments, the prostate cancer is AR antagonist resistant prostate cancer which overexpresses the glucocorticoid receptor (GR). In some embodiments, activation of the GR provides support for growth of the prostate cancer and/or confers antiandrogen resistance to the prostate cancer. In some embodiments, SARDs of this invention can be used to treat GR-dependent or GR-overexpressing prostate cancers, whether antiandrogen resistant or not. In some embodiments, SARDs of this invention can be used to treat PR-dependent or PR-overexpressing prostate cancers, whether antiandrogen resistant or not. In some embodiments, activation of GR and/or PR leads to reactivation of the AR-axis, which is preventable or treatable by use of the SARDs of this invention.

Yet another embodiment of the invention encompasses a method of treating prostate or other AR-expressing cancers using a SARD compound of the invention wherein the other cancers are selected from breast cancer such as triple negative breast cancer (TNBC), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer. In another embodiment, the breast cancer is triple negative breast cancer (TNBC).

The invention encompasses a method of reducing the levels of AR-splice variants in a subject comprising administering to the subject a therapeutically effective amount of a compound of this invention, or its isomer, optical isomer, or any mixture of optical isomers, pharmaceutically acceptable salt, pharmaceutical product, polymorph, hydrate or any combination thereof. The method may comprise further reducing the levels of AR-full length in the subject.

Another embodiment of the invention encompasses a method of treating Kennedy's disease in a subject comprising administering to the subject a compound of formulas 48-51, 53-58, 99A-99H, 120, 1072-1076, 1078-1080, and 1082-1094.

Yet another embodiment of the invention encompasses a method of: (a) treating acne in a subject, e.g., acne vulgaris; (b) decreasing sebum production in a subject, e.g., treats sehorrhea, seborrheic dermatitis, or acne; (c) treating hirsutism in a subject, e.g., female facial hair; (d) treating alopecia in a subject, e.g., androgenic alopecia, alopecia areata, alopecia secondary to chemotherapy, alopecia secondary to radiation therapy, alopecia induced by scarring, or alopecia induced by stress; (e) treating a hormonal condition in female, e.g., precocious puberty, early puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, or vaginal dryness; (f) treating sexual perversion, hypersexuality, or paraphilias in a subject; (g) treating androgen psychosis in a subject; (h) treating virilization in a subject; (i) treating complete or partial androgen insensitivity syndrome in a subject; (j) increasing or modulating ovulation in an animal; (k) treating of cancer in a subject; or any combination thereof, by administering a compound of this invention or a pharmaceutical composition thereof.

One embodiment of the invention encompasses methods of reducing the levels of polyglutamine (polyQ) AR polymorphs in a subject comprising administering a compound according to this invention. The method may inhibit, degrade, or both the function of the polyglutamine (polyQ) AR polymorphs (polyQ-AR). The polyQ-AR may be a short polyQ polymorph or a long polyQ polymorph. When the polyQ-AR is a short polyQ polymorph, the method further treats dermal disease. When the polyQ-AR is a long polyQ polymorph, the method further treats Kennedy's disease.

Another embodiment of the invention encompasses methods of treating amyotrophic lateral sclerosis (ALS) in a subject by administering a therapeutically effective amount of the compound of the invention, or its isomer, optical isomer, or any mixture of optical isomers, pharmaceutically acceptable salt, pharmaceutical product, polymorph, hydrate or any combination thereof; or a pharmaceutical composition thereof.

Another embodiment of the invention encompasses methods of treating abdominal aortic aneurysm (AAA) in a subject by administering a therapeutically effective amount of the compound of the invention, or its isomer, optical isomer, or any mixture of optical isomers, pharmaceutically acceptable salt, pharmaceutical product, polymorph, hydrate or any combination thereof; or a pharmaceutical composition thereof.

Yet another embodiment of the invention encompasses methods of treating uterine fibroids in a subject by administering a therapeutically effective amount of the compound of this invention, or its isomer, optical isomer, or any mixture of optical isomers, pharmaceutically acceptable salt, pharmaceutical product, polymorph, hydrate or any combination thereof; or a pharmaceutical composition thereof.

In yet another aspect, the invention provides a method of treating, suppressing, reducing the incidence, reducing the severity, or inhibiting the progression of a hormonal condition in a male in need thereof, comprising administering to the subject a therapeutically effective amount of a selective androgen receptor degrader (SARD) compound of the invention. In one embodiment, the condition in the method of the invention is hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 depicts that compound 48 exhibited 2- to 4-fold unexpectedly improved wtAR inhibition potency compared to 30 and 15.

FIG. 2 depicts that the wtAR inhibitory potency of 49 (3-formyl) was improved relative to 3-methyl (31), 3-carboxylic acid (15), and 3-COOEt (30).

FIG. 3 depicts that introduction of the oxime into 11 or 31 to produce 50 unexpectedly enhanced potency by 3- and 15-fold, respectively.

FIG. 4 depicts that unexpectedly, replacement of the 3-F (44) and 3-Cl (45) groups with a 3-CN (54) enhanced the in vitro potency by 10- and 15-fold, respectively.

FIG. 5 depicts that introduction of the oxime into 47 to produce 55 was tolerated, producing equipotent in vitro potency.

FIG. 6 depicts that replacement of the 3-COOH (15), 3-F (44), 3-COOH (15), and 3-COOEt (30) with a 3-NO2 (57) increased in vitro potency by at least 5-fold.

FIGS. 7A-7C depict the compounds of the invention inhibited R1881-induced wtAR transactivation. FIG. 7A was a positive control with agonist R1881; 56 and 54 were compared to enzalutamide (a standard LBD targeted agent) (FIG. 7B); and 56 had no agonist activity (assay in the absence of R1881) (FIG. 7C). AR transactivation method: COST cells were plated in 24 well plates at 40,000 cells/well in DME+5% csFBS without phenol red. Twenty-four hours after plating, the cells were transfected with 0.25 μg GRE-LUC, 0.01 μg CMV-LUC, 0.025 μg CMV-hAR using Lipofectamine reagents in optiMEM medium. Twenty-four hours after transfection, the cells were treated with a dose-response of the compounds in the presence of 0.1 nM R1881. Twenty-four hours after treatment, the cells were harvested, and luciferase assay was performed using Dual-luciferase reagent. Firefly values were divided by Renilla numbers and the values are represented as relative light units (RLU). The inhibition values are expressed as IC₅₀ values. For agonist activity, the cells were treated in the absence of R1881.

FIG. 8 depicts the IC₅₀ values for 54, 50, and 55 relative to enzalutamide.

FIGS. 9A and 9B depict the IC₅₀ values of compounds 54, 50, and 55 relative to enzalutamide (FIG. 9A) and demonstrated the absence of agonist activity for all three compounds (FIG. 9B).

FIG. 10 depicts the IC₅₀ values for 49, 50, and 53 relative to enzalutamide.

FIG. 11 depicts that 49 had a half life of ˜36 min in an in vitro stability in an in vitro rat liver microsomes (RLM) study.

FIG. 12 depicts that 50 was stable with a half-life that is in excess of 60 min in an in vitro RLM stability study.

FIG. 13 depicts that 49 was more stable in mouse liver microsomes (MLM) than RLM, with an MLM phase II half-life of about 55 minutes in an in vitro stability in MLM study.

FIG. 14 depicts the inhibition of AR transactivation and IC₅₀ values of 51, and 1082 compared to 1065.

FIG. 15 depicts the inhibition of transactivation and IC₅₀ values in a separate experiment for 1082 and 51 compared with 1065.

FIG. 16 depicts that 1074 and 1075 inhibited wtAR.

FIGS. 17A and 17B depict significant SARD activity of 1074, 1075, 1072, and 1076. FIG. 17A demonstrated that AR FL was degraded in LNCaP cells and FIG. 17B: demonstrated that AR SV was degraded in 22RV1 cells expressing both AR FL and AR SV.

FIG. 18 depicts AR transactivation results of 99D.

FIG. 19 depicts AR transactivation results of 99C.

FIG. 20 depicts AR transactivation results of 99A and 99B.

FIG. 21 depicts AR transactivation results of 57.

FIG. 22 depicts AR transactivation results of 99E.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Androgens act in cells by binding to the AR, a member of the steroid receptor superfamily of transcription factors. As the growth and maintenance of prostate cancer (PCa) is largely controlled by circulating androgens, treatment of PCa heavily relies on therapies that target AR. Treatment with AR antagonists such as darolutamide, enzalutamide, abiraterone (an indirect AR antagonist; others are LBD binding direct AR antagonists), apalutamide, bicalutamide or hydroxyflutamide to disrupt receptor activation has been successfully used in the past to reduce PCa growth. All currently available direct AR antagonists competitively bind AR and recruit corepressors such as NCoR and SMRT to repress transcription of target genes. However, altered intracellular signaling, AR mutations, and increased expression of coactivators lead to functional impairment of antagonists or even transformation of antagonists into agonists.

Studies have demonstrated that mutation of W741 and T877 within AR converts bicalutamide and hydroxyflutamide, respectively, to agonists. Similarly, increased intracellular cytokines recruit coactivators instead of corepressors to AR-responsive promoters subsequently converting bicalutamide to an agonist. Similarly, mutations that have been linked to enzalutamide, apalutamide, and abiraterone resistance include F876, H874, T877, and di-mutants T877/5888, T877/D890, F876/T877 (i.e., MR49 cells), and H874/T877 (Genome Biol. (2016) 17:10 (doi: 10.1186/s13059-015-0864-1)).

Abiraterone resistance mutations include L702H mutations which results in activation of the AR by glucocorticoids such as prednisone, causing resistance to abiraterone because abiraterone is usually prescribed in combination with prednisone. If resistance develops to enzalutamide or apalutamide then often the patient is refractory to abiraterone also and vice versa; or the duration of response is very short. Darolutamide also has limited efficacy and duration of action in CRPC. This situation highlights the need for a definitive androgen ablation therapy to prevent AR reactivation in advanced prostate cancers. Arora et al. in Cell 155, 1309-1322 reported the induction of glucocorticoid receptor (GR) expression as a common feature of drug-resistant tumors from prostate cancer cell lines (LNCaP/AR) and clinical samples. GR substituted for the AR to activate a similar but distinguishable set of target genes and was necessary for maintenance of the resistant phenotype. The GR agonist dexamethasone was sufficient to confer enzalutamide (or apalutamide) resistance, whereas a GR antagonist restored sensitivity. Acute AR inhibition resulted in GR upregulation in a subset of prostate cancer cells due to relief of AR-mediated feedback repression of GR expression. These findings establish a mechanism of escape from AR blockade through expansion of cells primed to drive AR target genes via an alternative nuclear receptor upon drug exposure. In some cases, the SARDs of this invention are potent GR antagonists in addition to potent AR antagonists. As such, they would possibly prevent the emergence of GR-dependent antiandrogen resistance or treat antiandrogen resistant prostate cancers which are dependent on GR. Though specific AR mutations or AR bypass mechanisms for conferring resistance to darolutamide have not yet been reported, darolutamide binds to the same LBD target on AR and resistance mutations are likely to develop that will be sensitive to the SARDs of this invention,

Despite initial response to androgen deprivation therapy (ADT), PCa disease progression is inevitable and the cancer emerges as castration-resistant prostate cancer (CRPC). The primary reason for castration resistant prostate cancer (CRPC) re-emergence is re-activation of androgen receptor (AR) by alternate mechanisms such as:
(a) intracrine androgen synthesis;
(b) expression of AR splice variants (AR-SV), e.g., that lack ligand binding domain (LBD);
(c) AR-LBD mutations with potential to resist antagonists;
(d) hyper-sensitization of AR to low androgen levels, e.g., due to AR gene amplification or AR mutation;
(e) amplification of the AR gene within the tumor; and
(f) over expression of coactivators and/or altered intracellular signal transduction.

The invention encompasses novel selective androgen receptor degrader (SARD) compounds encompassed by 48-51, 53-58, 99A-99H, 120, 1072-1076, 1078-1080, and 1082-1094, which inhibit the growth of prostate cancer (PCa) cells and tumors that are dependent on AR full length (AR-FL) including pathogenic and resistance mutations and wildtype, and/or AR splice variants (AR-SV) for proliferation.

As used herein, unless otherwise defined, a “selective androgen receptor degrader” (SARD) compound is an androgen receptor antagonist capable of inhibiting the growth of PCa cells and tumors that are dependent on AR-full length (AR-FL) and/or AR splice variants (AR-SV) for proliferation. The SARD compound may not bind to ligand binding domain (LBD). Alternatively, a “selective androgen receptor degrader” (SARD) compound is an androgen receptor antagonist capable of causing degradation of a variety of pathogenic mutant variant AR's and wildtype AR and hence are capable of exerting anti-androgenism is a wide variety of pathogenic altered cellular environments found in the disease states embodied in this invention. In one embodiment, the SARD is orally active. In another embodiment, the SARD is applied topically to the site of action.

The SARD compound may bind to the N-terminal domain (NTD) of the AR; to an alternate binding and degradation domain (BDD) of the AR; to both the AR ligand binding domain (LBD) and to an alternate binding and degradation domain (BDD); or to both the N-terminal domain (NTD) and to the ligand binding domain (LBD) of the AR. In one embodiment, the BDD may be located in the NTD. In one embodiment, the BDD is located in the AF-1 region of the NTD. Alternatively, the SARD compound may be capable of: inhibiting growth driven by the N-terminal domain (NTD)-dependent constitutively active AR-SV; or inhibiting the AR through binding to a domain that is distinct from the AR LBD. Also, the SARD compound may be a strong (i.e., highly potent and highly efficacious) selective androgen receptor antagonist, which antagonizes the AR stronger than other known AR antagonists (e.g., darolutamide, enzalutamide, apalutamide, bicalutamide and abiraterone).

The SARD compound may be a selective androgen receptor antagonist, which targets AR-SVs, which cannot be inhibited by conventional antagonists. The SARD compound may exhibit any one of several activities including, but not limited to: AR-SV degradation activity; AR-FL degradation activity; AR-SV inhibitory activity (i.e., is an AR-SV antagonist); AR-FL inhibitory activity (i.e., is an AR-FL antagonist); inhibition of the constitutive activation of AR-SVs; or inhibition of the constitutive activation of AR-FLs. Alternatively, the SARD compound may possess dual AR-SV degradation and AR-SV inhibitory functions, and/or dual AR-FL degradation and AR-FL inhibitory functions; or alternatively possess all four of these activities.

The SARD compound may also degrade AR-FL and AR-SV. The SARD compound may degrade the AR through binding to a domain that is distinct from the AR LBD. The SARD compound may possess dual degradation and AR-SV inhibitory functions that are distinct from any available CRPC therapeutics. The SARD compound may inhibit the re-activation of the AR by alternate mechanisms such as: intracrine androgen synthesis, expression of AR-SV that lack ligand binding domain (LBD) and AR-LBD mutations with potential to resist antagonists, or inhibit re-activated androgen receptors present in pathogenic altered cellular environments.

Examples of AR-splice variants include, but are not limited to, AR-V7 and ARv567es (a.k.a. AR-V12; S. Sun, et al. Castration resistance in human prostate cancer is conferred by a frequently occurring androgen receptor splice variant. J Clin Invest. (2010) 120(8), 2715-2730). Nonlimiting examples of AR mutations conferring antiandrogen resistance are: W741L, T877A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029) mutations. Many other LBD resistance conferring mutations are known in the art and will continue to be discovered. AR-V7 is a splice variant of AR that lacks the LBD (A. H. Bryce & E. S. Antonarakis. Androgen receptor splice variant 7 in castration-resistant prostate cancer: Clinical considerations. Int J Urol. (2016 Jun. 3) 23(8), 646-53. doi: 10.1111/iju.13134). It is constitutively active and has been demonstrated to be responsible for aggressive PCa and resistance to endocrine therapy.

The invention encompasses novel selective androgen receptor degrader (SARD) compounds of formulas 48-51, 53-58, 99A-99H, 120, 1072-1076, 1078-1080, and 1082-1094 which bind to the AR through an alternate binding and degradation domain (BDD), e.g., the NTD or AF-1. The SARDs may further bind the AR ligand binding domain (LBD).

The SARD compounds may be used in treating CRPC that cannot be treated with any other antagonist. The SARD compounds may treat CRPC by degrading AR-SVs. The SARD compounds may maintain their antagonistic activity in AR mutants that normally convert AR antagonists to agonists. For instance, the SARD compounds maintain their antagonistic activity to AR mutants W741L, T877A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029). Alternatively, the SARD compounds elicit antagonistic activity within an altered cellular environment in which LBD-targeted agents are not effective or in which NTD-dependent AR activity is constitutively active. Alternatively, SARD compounds can be co-antagonists of AR and GR and thereby overcome or prevent antiandrogen resistant CRPC in which GR is overexpressed and/or GR is activating the AR axis. Alternatively, SARD compounds are co-antagonists of AR and PR and thereby overcome or prevent antiandrogen resistant CRPC in which PR is overexpressed and/or PR is activating the AR axis.

### Selective Androgen Receptor Degrader (SARD) Compounds

The invention encompasses selective androgen receptor degrader (SARD) compounds selected from any one of the following structures:

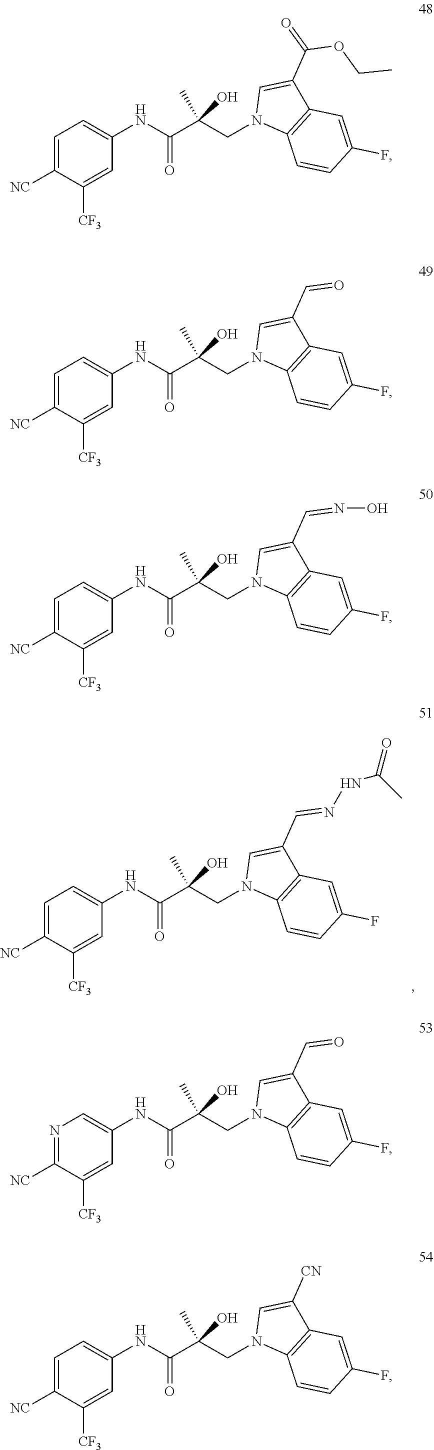 [SMILES: CCOC(=O)c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1ccc(C#N)c(C(F)(F)F)c1 ; C[C@](O)(Cn1cc(/C=N\O)c2cc(F)ccc21)C(=O)Nc1ccc(C#N)c(C(F)(F)F)c1 ; CC(=O)N/N=C/c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1cnc(C#N)c(C(F)(F)F)c1 ; C[C@](O)(Cn1cc(C#N)c2cc(F)ccc21)C(=O)Nc1ccc(C#N)c(C(F)(F)F)c1]

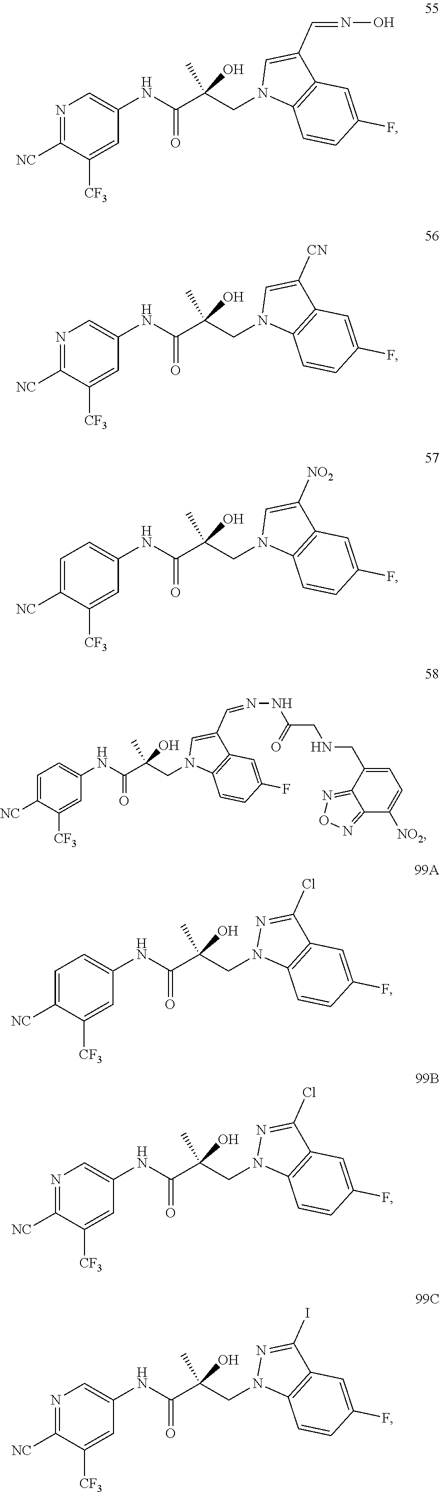

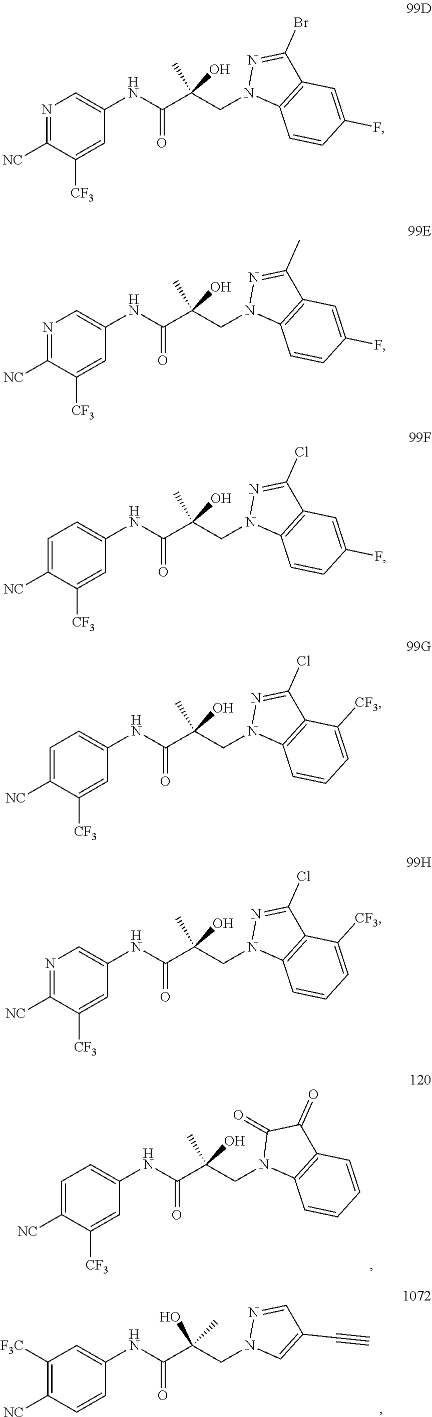

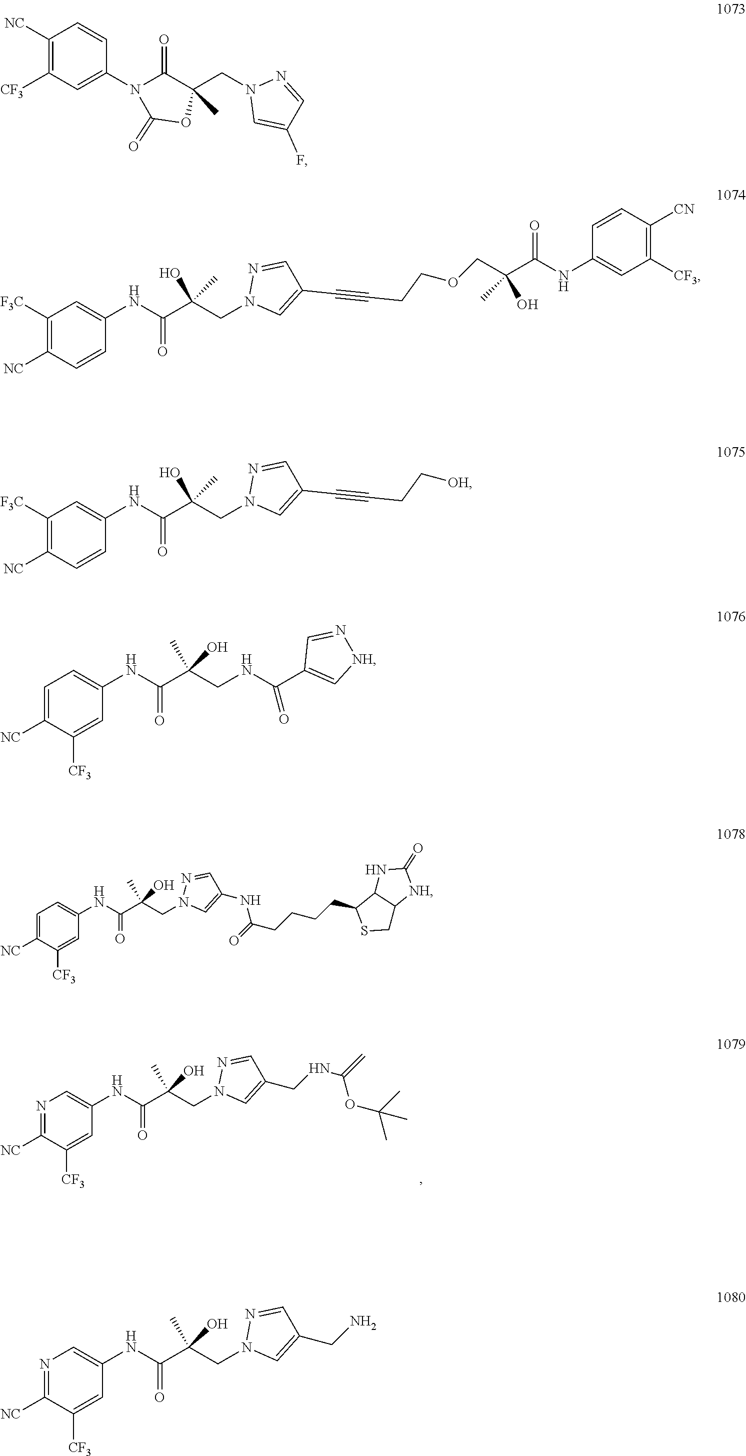

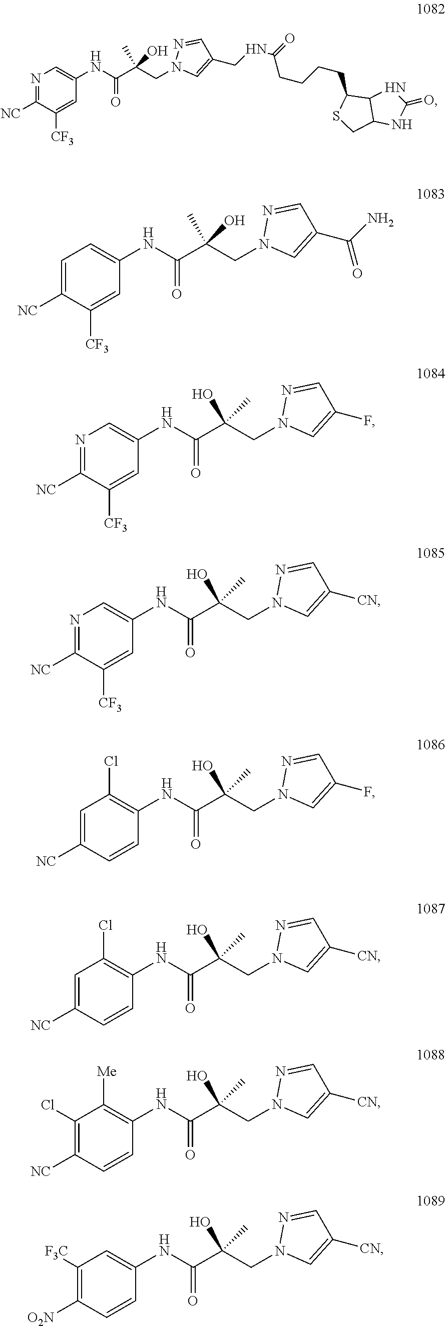

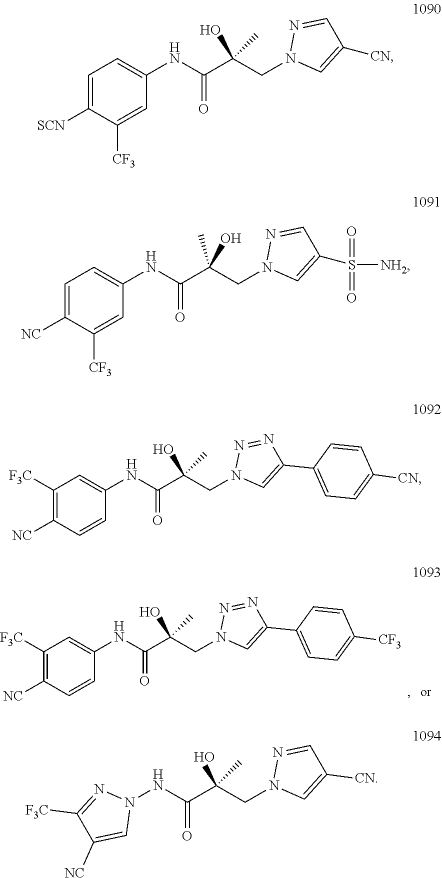

In one embodiment, this invention provides the compounds and/or its use and/or its derivative, optical isomer, mixtures of optical isomers including racemates, isomer, metabolite, pharmaceutically acceptable salt, pharmaceutical product, hydrate, N-oxide, prodrug, polymorph, crystal or combinations thereof.

In one embodiment, the methods of this invention make use of “pharmaceutically acceptable salts” of the compounds, which may be produced, by reaction of a compound of this invention with an acid or base.

The compounds of the invention may be converted into pharmaceutically acceptable salts. A pharmaceutically acceptable salt may be produced by reaction of a compound with an acid or base.

Suitable pharmaceutically acceptable salts of amines may be prepared from an inorganic acid or from an organic acid. Examples of inorganic salts of amines include, but are not limited to, bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates, or thiocyanates.

Examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxylates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates, gluconates, glutamates, glycolates, glucorates, glucoheptanoates, glycerophosphates, gluceptates, glycollylarsanilates, glutarates, glutamates, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, nitrates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, pectinates, phenylpropionates, palmitates, pantothenates, polygalacturates, pyruvates, quinates, salicylates, succinates, stearates, sulfanilates, subacetates, tartarates, theophyllineacetates, p-toluenesulfonates (tosylates), trifluoroacetates, terephthalates, tannates, teoclates, trihaloacetates, triethiodide, tricarboxylates, undecanoates and valerates. Examples of inorganic salts of carboxylic acids or phenols may be selected from ammonium, alkali metals, and alkaline earth metals. Alkali metals include, but are not limited to, lithium, sodium, potassium, or cesium. Alkaline earth metals include, but are not limited to, calcium, magnesium, aluminium; zinc, barium, cholines, or quaternary ammoniums. Examples of organic salts of carboxylic acids or phenols may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglumines, N-methyl-D-glucamines, N,N′-dibenzylethylenediamines, nicotinamides, organic amines, ornithines, pyridines, picolines, piperazines, procaine, tris(hydroxymethyl)methylamines, triethylamines, triethanolamines, trimethylamines, tromethamines and ureas.

In various embodiments, the pharmaceutically acceptable salts of the compounds of this invention include: HCl salt, oxalic acid salt, L-(+)-tartaric acid salt, HBr salt and succinic acid salt. Each represents a separate embodiment of this invention.

Salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

The methods of the invention may use an uncharged compound or a pharmaceutically acceptable salt of the compound. In particular, the methods use pharmaceutically acceptable salts of compounds of formulas 48-51, 53-58, 99A-99H, 120, 1072-1076, 1078-1080, and 1082-1094. The pharmaceutically acceptable salt may be an amine salt or a salt of a phenol of the compounds of formulas 48-51, 53-58, 99A-99H, 120, 1072-1076, 1078-1080, and 1082-1094.

In one embodiment, the methods of this invention make use of a free base, free acid, non charged or non-complexed compounds of formulas 48-51, 53-58, 99A-99H, 120, 1072-1076, 1078-1080, and 1082-1094, and/or its isomer, optical isomer, or any mixture of optical isomers, pharmaceutical product, hydrate, polymorph, or combinations thereof.

In one embodiment, the methods of this invention make use of an optical isomer of a compound of formulas 48-51, 53-58, 99A-99H, 120, 1072-1076, 1078-1080, and 1082-1094. In one embodiment, the methods of this invention make use of an isomer of a compound of formulas 48-51, 53-58, 99A-99H, 120, 1072-1076, 1078-1080, and 1082-1094. In one embodiment, the methods of this invention make use of a pharmaceutical product of a compound of formulas. In one embodiment, the methods of this invention make use of a hydrate of a compound of formulas. In one embodiment, the methods of this invention make use of a polymorph of a compound of formulas. In one embodiment, the methods of this invention make use of a metabolite of a compound of formulas. In another embodiment, the methods of this invention make use of a composition comprising a compound of formulas, as described herein, or, in another embodiment, a combination of isomer, metabolite, pharmaceutical product, hydrate, polymorph of a compound of formulas.

As used herein, the term “isomer” includes, but is not limited to, optical isomers, structural isomers, or conformational isomers.

The term “isomer” is meant to encompass optical isomers of the SARD compound. It will be appreciated by those skilled in the art that the SARDs of the present invention contain at least one chiral center. Accordingly, the compounds may exist as optically-active (such as an (R) isomer or (S) isomer) or racemic forms. Optically active compounds may exist as enantiomerically enriched mixtures. Some compounds may also exhibit polymorphism. It is to be understood that the present invention encompasses any racemic, optically active, polymorphic, or stereroisomeric form, or mixtures thereof. Thus, the invention may encompass SARD compounds as pure (R)-isomers or as pure (S)-isomers. It is known in the art how to prepare optically active forms. For example, by resolution of the racemic form by recrystallization techniques, by synthesis from optically active starting materials, by chiral synthesis, or by chromatographic separation using a chiral stationary phase.

Compounds of the invention may be hydrates of the compounds. As used herein, the term “hydrate” includes, but is not limited to, hemihydrate, monohydrate, dihydrate, or trihydrate. The invention also includes use of N-oxides of the amino substituents of the compounds described herein.

This invention provides, in other embodiments, use of metabolites of the compounds as herein described. In one embodiment, “metabolite” means any substance produced from another substance by metabolism or a metabolic process.

In one embodiment, the compounds of this invention are prepared as described herein, e.g., according to Example 1.

### Biological Activity of Selective Androgen Receptor Degraders

In one embodiment, this invention provides a method of treating prostate cancer (PCa) or increasing the survival of a male subject suffering from prostate cancer comprising administering to the subject a therapeutically effective amount of a compound or its pharmaceutically acceptable salt, or isomer, represented by a compound of formulas 48-51, 53-58, 99A-99H, 120, 1072-1076, 1078-1080, and 1082-1094.

The prostate cancer may be advanced prostate cancer, refractory prostate cancer, castration resistant prostate cancer (CRPC), metastatic CRPC (mCRPC), non-metastatic CRPC (nmCRPC), high-risk nmCRPC or any combination thereof.

The prostate cancer may depend on AR-FL and/or AR-SV for proliferation. The prostate or other cancer may be resistant to treatment with an androgen receptor antagonist. The prostate or other cancer may be resistant to treatment with darolutamide, enzalutamide, apalutamide, bicalutamide, abiraterone, EPI-001, EPI-506, AZD-3514, galeterone, ASC-J9, flutamide, hydroxyflutamide, nilutamide, cyproterone acetate, ketoconazole, spironolactone, or any combination thereof. The method may also reduce the levels of AR, AR-FL, AR-FL with antiandrogen resistance-conferring AR-LBD mutations, AR-SV, gene-amplified AR, or any combination thereof.

In one embodiment, this invention provides a method of treating enzalutamide resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

In one embodiment, this invention provides a method of treating apalutamide resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

In one embodiment, this invention provides a method of treating abiraterone resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

The invention encompasses a method of treating or inhibiting the progression of apalutamide resistant prostate cancer (PCa) or increasing the survival of a male subject suffering from apalutamide resistant prostate cancer comprising administering to the subject a therapeutically effective amount of a SARD compound or pharmaceutically acceptable salt, wherein the compound is represented by a compound of formulas 48-51, 53-58, 99A-99H, 120, 1072-1076, 1078-1080, and 1082-1094.

In one embodiment, this invention provides a method of treating darolutamide resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

The invention encompasses a method of treating or inhibiting the progression of darolutamide resistant prostate cancer (PCa) or increasing the survival of a male subject suffering from apalutamide resistant prostate cancer comprising administering to the subject a therapeutically effective amount of a SARD compound or pharmaceutically acceptable salt, wherein the compound is represented by a compound of formulas 48-51, 53-58, 99A-99H, 120, 1072-1076, 1078-1080, and 1082-1094.

The method may further comprise administering androgen deprivation therapy to the subject.

In one embodiment, this invention provides a method of treating triple negative breast cancer (TNBC) comprising administering to the subject a therapeutically effective amount of a compound of this invention, or its optical isomer, isomer, pharmaceutically acceptable salt, pharmaceutical product, polymorph, hydrate or any combination thereof.

The method may further comprise a second therapy such as androgen deprivation therapy (ADT) or LHRH agonist or antagonist. LHRH agonists include, but are not limited to, leuprolide acetate.

The invention encompasses a method of treating or inhibiting the progression of prostate cancer (PCa) or increasing the survival of a male subject suffering from prostate cancer comprising administering to the subject a therapeutically effective amount of a SARD compound or pharmaceutically acceptable salt, wherein the compound is at least one of compounds 48-51, 53-58, 99A-99H, 120, 1072-1076, 1078-1080, and 1082-1094.

The invention encompasses a method of treating or inhibiting the progression of refractory prostate cancer (PCa) or increasing the survival of a male subject suffering from refractory prostate cancer comprising administering to the subject a therapeutically effective amount of a SARD compound or pharmaceutically acceptable salt, wherein the compound is represented by a compound of formulas 48-51, 53-58, 99A-99H, 120, 1072-1076, 1078-1080, and 1082-1094.

The invention encompasses a method of treating or increasing the survival of a male subject suffering from castration resistant prostate cancer (CRPC) comprising administering to the subject a therapeutically effective amount of a SARD wherein the compound is represented by a compound of formulas 48-51, 53-58, 99A-99H, 120, 1072-1076, 1078-1080, and 1082-1094.

The method may further comprise administering androgen deprivation therapy to the subject.

The invention encompasses a method of treating or inhibiting the progression of enzalutamide resistant prostate cancer (PCa) or increasing the survival of a male subject suffering from enzalutamide resistant prostate cancer comprising administering to the subject a therapeutically effective amount of a SARD compound or pharmaceutically acceptable salt, wherein the compound is represented by a compound of formulas 48-51, 53-58, 99A-99H, 120, 1072-1076, 1078-1080, and 1082-1094.

The method may further comprise administering androgen deprivation therapy to the subject.

The invention encompasses a method of treating or inhibiting the progression of triple negative breast cancer (TNBC) or increasing the survival of a female subject suffering from triple negative breast cancer comprising administering to the subject a therapeutically effective amount of a SARD compound or pharmaceutically acceptable salt, wherein the compound is represented by a compound of formulas 48-51, 53-58, 99A-99H, 120, 1072-1076, 1078-1080, and 1082-1094.

As used herein, the term “increase the survival” refers to a lengthening of time when describing the survival of a subject. Thus in this context, the compounds of the invention may be used to increase the survival of men with advanced prostate cancer, refractory prostate cancer, castration resistant prostate cancer (CRPC); metastatic CRPC (mCRPC); non-metastatic CRPC (nmCRPC); or high-risk nmCRPC; or women with TNBC.

Alternatively, as used herein, the terms “increase”, “increasing”, or “increased” may be used interchangeably and refer to an entity becoming progressively greater (as in size, amount, number, or intensity), wherein for example the entity is sex hormone-binding globulin (SHBG) or prostate-specific antigen (PSA).

The compounds and compositions of the invention may be used for increasing metastasis-free survival (MFS) in a subject suffering from non-metastatic prostate cancer. The non-metastatic prostate cancer may be non-metastatic advanced prostate cancer, non-metastatic CRPC (nmCRPC), or high-risk nmCRPC.

The SARD compounds described herein may be used to provide a dual action. For example, the SARD compounds may treat prostate cancer and prevent metastasis. The prostate cancer may be refractory prostate cancer; advanced prostate cancer; castration resistant prostate cancer (CRPC); metastatic CRPC (mCRPC); non-metastatic CRPC (nmCRPC); or high-risk nmCRPC.

The SARD compounds described herein may be used to provide a dual action. For example, the SARD compounds may treat TNBC and prevent metastasis.

Men with advanced prostate cancer who are at high risk for progression to castration resistant prostate cancer (CRPC) are men on ADT with serum total testosterone concentrations greater than 20 ng/dL or men with advanced prostate cancer who at the time of starting ADT had either (1) confirmed Gleason pattern 4 or 5 prostate cancer, (2) metastatic prostate cancer, (3) a PSA doubling time<3 months, (4) a PSA>20 ng/mL, or (5) a PSA relapse in <3 years after definitive local therapy (radical prostatectomy or radiation therapy).

Normal levels of prostate specific antigen (PSA) are dependent on several factors, such as age and the size of a male subject's prostate, among others. PSA levels in the range between 2.5-10 ng/mL are considered “borderline high” while levels above 10 ng/mL are considered “high.” A rate change or “PSA velocity” greater than 0.75/year is considered high. PSA levels may increase despite ongoing ADT or a history of ADT, surgical castration or despite treatment with antiandrogens and/or LHRH agonist.

Men with high risk non-metastatic castration resistant prostate cancer (high-risk nmCRPC) may include those with rapid PSA doubling times, having an expected progression-free survival of approximately 18 months or less (Miller K, Moul JW, Gleave M, et al.2013. “Phase III, randomized, placebo-controlled study of once-daily oral zibotentan (ZD4054) in patients with non-metastatic castration-resistant prostate cancer,” Prostate Canc Prost Dis. February; 16:187-192). This relatively rapid progression of their disease underscores the importance of novel therapies for these individuals.

The methods of the invention may treat subjects with PSA levels greater than 8 ng/mL where the subject suffers from high-risk nmCRPC. The patient population includes subjects suffering from nmCRPC where PSA doubles in less than 8 months or less than 10 months. The method may also treat patient populations where the total serum testosterone levels are greater than 20 ng/mL in a subject suffering from high-risk nmCRPC. In one case, the serum free testosterone levels are greater than those observed in an orchiectomized male in a subject suffering from high-risk nmCRPC.

The pharmaceutical compositions of the invention may further comprise at least one LHRH agonist or antagonist, antiandrogen, anti-programmed death receptor 1 (anti-PD-1) drug or anti-PD-L1 drug. LHRH agonists include, but are not limited to, leuprolide acetate (Lupron®) (U.S. Pat. Nos. 5,480,656; 5,575,987; 5,631,020; 5,643,607; 5,716,640; 5,814,342; 6,036,976 hereby incorporated by reference) or goserelin acetate (Zoladex®) (U.S. Pat Nos. 7,118,552; 7,220,247; 7,500,964 hereby incorporated by reference). LHRH antagonists include, but are not limited to, degarelix or abarelix. Antiandrogens include, but are not limited to, bicalutamide, flutamide, finasteride, dutasteride, darolutamide, enzalutamide, apalutamide, nilutamide, chlormadinone, abiraterone, or any combination thereof. Anti-PD-1 drugs include, but are not limited to, AMP-224, nivolumab, pembrolizumab, pidilizumab, and AMP-554. Anti-PD-L1 drugs include, but are not limited to, BMS-936559, atezolizumab, durvalumab, avelumab, and MPDL3280A. Anti-CTLA-4 drugs include, but are not limited to, ipilimumab and tremelimumab.

Treatment of prostate cancer, advanced prostate cancer, CRPC, mCRPC and/or nmCRPC may result in clinically meaningful improvement in prostate cancer related symptoms, function and/or survival. Clinically meaningful improvement can be determined by an increase in radiographic progression free survival (rPFS) if cancer is metastatic, or an increase metastasis-free survival (MFS) if cancer is non-metastatic, among others.

The invention encompasses methods of lowering serum prostate specific antigen (PSA) levels in a male subject suffering from prostate cancer, advanced prostate cancer, metastatic prostate cancer or castration resistant prostate cancer (CRPC) comprising administering a therapeutically effective amount of a SARD compound, wherein the compound is represented by the structure of formulas 48-51, 53-58, 99A-99H, 120, 1072-1076, 1078-1080, and 1082-1094.

The invention encompasses a method of secondary hormonal therapy that reduces serum PSA in a male subject suffering from castration resistant prostate cancer (CRPC) comprising administering a therapeutically effective amount of a compound of formulas 48-51, 53-58, 99A-99H, 120, 1072-1076, 1078-1080, and 1082-1094 that reduces serum PSA in a male subject suffering from castration resistant prostate cancer.

The invention encompasses a method of reducing levels of AR, AR-full length (AR-FL), AR-FL with antiandrogen resistance-conferring AR-LBD mutations, AR-splice variant (AR-SV), and/or amplifications of the AR gene within the tumor in the subject in need thereof comprising administering a therapeutically effective amount of a compound of formulas 48-51, 53-58, 99A-99H, 120, 1072-1076, 1078-1080, and 1082-1094 to reduce the level of AR, AR-full length (AR-FL), AR-FL with antiandrogen resistance-conferring AR-LBD or other AR mutations, AR-splice variant (AR-SV), and/or amplifications of the AR gene within the tumor.

The method may increase radiographic progression free survival (rPFS) or metastasis-free survival (MFS).

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, or non-metastatic castration resistant prostate cancer (nmCRPC) patient. In these subjects, the refractory may be enzalutamide resistant prostate cancer. In these subjects, the nmCRPC may be high-risk nmCRPC. Further the subject may be on androgen deprivation therapy (ADT) with or without castrate levels of total T.

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, or non-metastatic castration resistant prostate cancer (nmCRPC) patient. In these subjects, the refractory PC may be apalutamide resistant prostate cancer. In these subjects, the nmCRPC may be high-risk nmCRPC. Further the subject may be on androgen deprivation therapy (ADT) with or without castrate levels of total T.

As used herein, the phrase “a subject suffering from castration resistant prostate cancer” refers to a subject with at least one of the following characteristics: has been previously treated with androgen deprivation therapy (ADT); has responded to the ADT and currently has a serum PSA>2 ng/mL or >2 ng/mL and representing a 25% increase above the nadir achieved on the ADT; a subject which despite being maintained on androgen deprivation therapy is diagnosed to have serum PSA progression; a castrate level of serum total testosterone (<50 ng/dL) or a castrate level of serum total testosterone (<20 ng/dL). The subject may have rising serum PSA on two successive assessments at least 2 weeks apart; been effectively treated with ADT; or has a history of serum PSA response after initiation of ADT.

As used herein, the term “serum PSA progression” refers to a 25% or greater increase in serum PSA and an absolute increase of 2 ng/ml or more from the nadir; or to serum PSA>2 ng/mL, or >2 ng/mL and a 25% increase above the nadir after the initiation of androgen deprivation therapy (ADT). The term “nadir” refers to the lowest PSA level while a patient is undergoing ADT.

The term “serum PSA response” refers to at least one of the following: at least 90% reduction in serum PSA value prior to the initiation of ADT; to <10 ng/mL undetectable level of serum PSA (<0.2 ng/mL) at any time; at least 50% decline from baseline in serum PSA; at least 90% decline from baseline in serum PSA; at least 30% decline from baseline in serum PSA; or at least 10% decline from baseline in serum PSA.

The methods of this invention comprise administering a combination of forms of ADT and a compound of this invention. Forms of ADT include a LHRH agonist. LHRH agonist includes, but is not limited to, leuprolide acetate (Lupron®) (U.S. Pat. Nos. 5,480,656; 5,575,987; 5,631,020; 5,643,607; 5,716,640; 5,814,342; 6,036,976 hereby incorporated by reference) or goserelin acetate (Zoladex®) (U.S. Pat. Nos. 7,118,552; 7,220,247; 7,500,964 hereby incorporated by reference). Forms of ADT include, but are not limited to LHRH antagonists, reversible antiandrogens, or bilateral orchidectomy. LHRH antagonists include, but are not limited to, degarelix and abarelix. Antiandrogens include, but are not limited to, bicalutamide, flutamide, hydroxyflutamide, finasteride, dutasteride, enzalutamide, apalutamide, EPI-001, EPI-506, darolutamide, nilutamide, chlormadinone, abiraterone, or any combination thereof.

The methods of the invention encompass administering at least one compound of the invention and a lyase inhibitor (e.g., abiraterone).

The term “advanced prostate cancer” refers to metastatic cancer having originated in the prostate, and having widely metastasized to beyond the prostate such as the surrounding tissues to include the seminal vesicles the pelvic lymph nodes or bone, or to other parts of the body. Prostate cancer pathologies are graded with a Gleason grading from 1 to 5 in order of increasing malignancy. Patients with significant risk of progressive disease and/or death from prostate cancer should be included in the definition and any patient with cancer outside the prostate capsule with disease stages as low as IIB clearly has “advanced” disease. “Advanced prostate cancer” can refer to locally advanced prostate cancer. Similarly, “advanced breast cancer” refers to metastatic cancer having originated in the breast, and having widely metastasized to beyond the breast to surrounding tissues or other parts of the body such as the liver, brain, lungs, or bone.

The term “refractory” may refer to cancers that do not respond to treatment. E.g., prostate or breast cancer may be resistant at the beginning of treatment or it may become resistant during treatment. “Refractory cancer” may also be referred to herein as “resistant cancer”.

The term “castration resistant prostate cancer” (CRPC) refers to advanced prostate cancer that is worsening or progressing while the patient remains on ADT or other therapies to reduce testosterone, or prostate cancer which is considered hormone refractory, hormone naive, androgen independent or chemical or surgical castration resistant. CRPC may be the result of AR activation by intracrine androgen synthesis; expression of AR splice variants (AR-SV) that lack ligand binding domain (LBD); or expression of AR-LBD or other AR mutations with potential to resist antagonists. Castration resistant prostate cancer (CRPC) is an advanced prostate cancer which developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, enzalutamide, apalutamide, darolutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®), cabozantinib (Cometrig™, also known as XL184)) or other prostate cancer therapies (e.g., vaccines (sipuleucel-T (Provenge®), GVAX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

Castration resistant prostate cancer may be defined as hormone naïve prostate cancer. In men with castration resistant prostate cancer, the tumor cells may have the ability to grow in the absence of androgens (hormones that promote the development and maintenance of male sex characteristics).

Many early prostate cancers require androgens for growth, but advanced prostate cancers are androgen-independent, or hormone naïve.

The term “androgen deprivation therapy” (ADT) may include orchiectomy; administering luteinizing hormone-releasing hormone (LHRH) analogs; administering luteinizing hormone-releasing hormone (LHRH) antagonists; administering 5a-reductase inhibitors; administering antiandrogens; administering inhibitors of testosterone biosynthesis; administering estrogens; or administering 17a-hydroxylase/C17,20 lyase (CYP17A1) inhibitors. LHRH drugs lower the amount of testosterone made by the testicles. Examples of LHRH analogs available in the United States include leuprolide (Lupron®, Viadur®, Eligard®), goserelin (Zoladex®), triptorelin (Trelstar®), and histrelin (Vantas®). Antiandrogens block the body's ability to use any androgens. Examples of antiandrogens drugs include darolutamide (Nubeqa®), enzalutamide (Xtandi®), apalutamide (Erleada®), flutamide (Eulexin®), bicalutamide (Casodex®), and nilutamide (Nilandron®). Luteinizing hormone-releasing hormone (LHRH) antagonists include abarelix (Plenaxis®) or degarelix (Firmagon®) (approved for use by the FDA in 2008 to treat advanced prostate cancer). 5a-Reductase inhibitors block the body's ability to convert testosterone to the more active androgen, 5a-dihydrotestosterone (DHT) and include drugs such as finasteride (Proscar®) and dutasteride (Avodart®). Inhibitors of testosterone biosynthesis include drugs such as ketoconazole (Nizoral®). Estrogens include diethylstilbestrol or 17b-estradiol. 17a-Hydroxylase/C17,20 lyase (CYP17A1) inhibitors include abiraterone (Zytiga®).

The invention encompasses a method of treating antiandrogen-resistant prostate cancer. The antiandrogen may include, but is not limited to, bicalutamide, hydroxyflutamide, flutamide, darolutamide, enzalutamide, apalutamide or abiraterone.

The invention encompasses a method of treating prostate cancer in a subject in need thereof, wherein said subject has a rearranged AR, AR overexpressing prostate cancer, castration-resistant prostate cancer, castration-sensitive prostate cancer, AR-V7 expressing prostate cancer, or d567ES expressing prostate cancer, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by at least one of a compound of formulas 48-51, 53-58, 99A-99H, 120, 1072-1076, 1078-1080, and 1082-1094.

In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

In one embodiment, the castration-sensitive prostate cancer is F876L mutation expressing castration-sensitive prostate cancer, F876L_T877A double mutation castration-sensitive prostate cancer, and/or castration-sensitive prostate cancer characterized by intratumoral androgen synthesis.

In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating AR overexpressing prostate cancer in a subject in need thereof, comprising administering to the subject a therapeutically effective amount of a selective androgen receptor degrader (SARD compound, or its isomer, pharmaceutically acceptable salt, pharmaceutical product, polymorph, hydrate or any combination thereof, wherein said SARD compound is represented by the at least one of compounds 48-51, 53-58, 99A-99H, 120, 1072-1076, 1078-1080, and 1082-1094.

The invention encompasses a method of treating castration-resistant prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is at least one of compounds 48-51, 53-58, 99A-99H, 120, 1072-1076, 1078-1080, and 1082-1094. In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

The invention encompasses a method of treating castration-sensitive prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is at least one of compounds 48-51, 53-58, 99A-99H, 120, 1072-1076, 1078-1080, and 1082-1094. In one embodiment, the castration-sensitive prostate cancer is F876L mutation expressing castration-sensitive prostate cancer, F876L_T877A double mutation castration-sensitive prostate cancer, and/or castration-sensitive prostate cancer characterized by intratumoral androgen synthesis. In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating AR-V7 expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by at least one of compounds 48-51, 53-58, 99A-99H, 120, 1072-1076, 1078-1080, and 1082-1094.

The invention encompasses a method of treating d567ES expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by at least one of compounds 48-51, 53-58, 99A-99H, 120, 1072-1076, 1078-1080, and 1082-1094. Treatment of Triple Negative Breast Cancer (TNBC)

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, we have evidence that certain TNBC's may be supported by androgen independent activation of AR-SVs lacking the LBD or androgen-dependent activation of AR full length. As such, darolutamide, enzalutamide, apalutamide, and other LBD-directed traditional AR antagonists would not be able to antagonize AR-SVs in these TNBC's. However, SARDs of this invention which are capable of destroying AR-SVs (see Table 1 and Example 2) through a binding site in the NTD of AR (see Example 9 of US2017-0368003) would be able to antagonize AR in these TNBC's and provide an anti-tumor effect, as shown in Example 8 of US2017-0368003.

### Treatment of Kennedy's Disease

Muscle atrophy (MA) is characterized by wasting away or diminution of muscle and a decrease in muscle mass. For example, post-polio MA is muscle wasting that occurs as part of the post-polio syndrome (PPS). The atrophy includes weakness, muscle fatigue, and pain. Another type of MA is X-linked spinal-bulbar muscular atrophy (SBMA—also known as Kennedy's Disease). This disease arises from a defect in the androgen receptor gene on the X chromosome, affects only males, and its onset is in late adolescence to adulthood. Proximal limb and bulbar muscle weakness results in physical limitations including dependence on a wheelchair in some cases. The mutation results in an extended polyglutamine tract at the N-terminal domain of the androgen receptor (polyQ AR).

Binding and activation of the polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci. 201658(3), 343-347). These steps are required for pathogenesis and result in partial loss of transactivation function (i.e., an androgen insensitivity) and a poorly understood neuromuscular degeneration. Peripheral polyQ AR anti-sense therapy rescues disease in mouse models of SBMA (Cell Reports 7, 774-784, May 8, 2014). Further support of use antiandrogen comes in a report in which the antiandrogen flutamide protects male mice from androgen-dependent toxicity in three models of spinal bulbar muscular atrophy (Renier K J, et al. Endocrinology 2014, 155(7), 2624-2634). These steps are required for pathogenesis and result in partial loss of transactivation function (i.e., an androgen insensitivity) and a poorly understood neuromuscular degeneration. Currently there are no disease-modifying treatments but rather only symptom directed treatments. Efforts to target the polyQ AR as the proximal mediator of toxicity by harnessing cellular machinery to promote its degradation hold promise for therapeutic intervention.

In another aspect, the invention provides a method of treating an androgen receptor dependent disease or condition or an androgen dependent disease or condition in a subject in need thereof, comprising administering a therapeutically effective amount of a compound of formulas 48-51, 53-58, 99A-99H, 120, 1072-1076, 1078-1080, and 1082-1094.

In one embodiment, the androgen receptor dependent disease or condition responds to at least one of AR-splice variant (AR-SV) degradation activity, full length (AR-FL) degradation activity, AR-SV inhibitory, or AR-FL inhibitory activity.

Selective androgen receptor degraders such as those reported herein bind to, inhibit transactivation, and degrade all androgen receptors tested to date (full length, splice variant, antiandrogen resistance mutants, etc.), indicating that they are promising leads for treatment diseases whose pathogenesis is androgen-dependent such as SBMA.

The invention encompasses methods of treating Kennedy's disease comprising administering a therapeutically effective amount of a compound of formulas 48-51, 53-58, 99A-99H, 120, 1072-1076, 1078-1080, and 1082-1094.

As used herein, the term “androgen receptor associated conditions” or “androgen sensitive diseases or disorders” or “androgen-dependent diseases or disorders” are conditions, diseases, or disorders that are modulated by or whose pathogenesis is dependent upon the activity of the androgen receptor. The androgen receptor is expressed in most tissues of the body however it is overexpressed in, inter alia, the prostate and skin. ADT has been the mainstay of prostate cancer treatment for many years, and SARDs may also be useful in treating various prostate cancers, benign prostatic hypertrophy, prostamegaly, and other maladies of the prostate. In one embodiment, an “androgen receptor dependent disease or condition” is a medical condition that is, in part or in full, dependent on, or is sensitive to, the presence of androgenic activity or activation of the AR-axis in the body. In one embodiment, an androgen dependent disease or condition is used interchangeably with and androgen receptor dependent disease or condition.

The invention encompasses methods of treating benign prostatic hypertrophy comprising administering a therapeutically effective amount of at least one compound of formulas 48-51, 53-58, 99A-99H, 120, 1072-1076, 1078-1080, and 1082-1094.

The invention encompasses methods of treating prostamegaly comprising administering a therapeutically effective amount of at least one compound of formulas 48-51, 53-58, 99A-99H, 120, 1072-1076, 1078-1080, and 1082-1094.

The invention encompasses methods of treating hyperproliferative prostatic disorders and diseases comprising administering a therapeutically effective amount of a compound of formulas 48-51, 53-58, 99A-99H, 120, 1072-1076, 1078-1080, and 1082-1094.

The effect of the AR on the skin is apparent in the gender dimorphism and puberty related dermatological problems common to teens and early adults. The hyperandrogenism of puberty stimulates terminal hair growth, sebum production, and predisposes male teens to acne, acne vulgaris, seborrhea, excess sebum, hidradenitis suppurativa, hirsutism, hypertrichosis, hyperpilosity, androgenic alopecia, male pattern baldness, and other dermatological maladies. Although antiandrogens theoretically should prevent the hyperandrogenic dermatological diseases discussed, they are limited by toxicities, sexual side effects, and lack of efficacy when topically applied. The SARDs of this invention potently inhibit ligand-dependent and ligand-independent AR activation, and (in some cases) have short biological half-lives in the serum, suggesting that topically formulated SARDs of this invention could be applied to the areas affected by acne, seborrheic dermatitis, and/or hirsutism without risk of systemic side effects.

The invention encompasses methods of treating acne, acne vulgaris, seborrhea, seborrheic dermatitis, hidradenitis supporativa, hirsutism, hypertrichosis, hyperpilosity, or alopecia comprising administering a therapeutically effective amount of a compound of formulas 48-51, 53-58, 99A-99H, 120, 1072-1076, 1078-1080, and 1082-1094.

The compounds and/or compositions described herein may be used for treating hair loss, alopecia, androgenic alopecia, alopecia areata, alopecia secondary to chemotherapy, alopecia secondary to radiation therapy, alopecia induced by scarring or alopecia induced by stress. Generally “hair loss” or “alopecia” refers to baldness as in the very common type of male-pattern baldness. Baldness typically begins with patch hair loss on the scalp and sometimes progresses to complete baldness and even loss of body hair. Hair loss affects both males and females.

The invention encompasses methods of treating androgenic alopecia comprising administering a therapeutically effective amount of a compound of formulas 48-51, 53-58, 99A-99H, 120, 1072-1076, 1078-1080, and 1082-1094.

SARDs of this invention may also be useful in the treatment of hormonal conditions in females which can have hyperandrogenic pathogenesis such as precocious puberty, early puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, and/or vaginal dryness. In another embodiment, the hormonal condition in females is an androgen-dependent hormonal condition in a female.

The invention encompasses methods of treating precocious puberty or early puberty, dysmenorrhea or amenorrhea, multilocular uterus syndrome, endometriosis, hysteromyoma, abnormal uterine bleeding, hyper-androgenic diseases (such as polycystic ovary syndrome (PCOS)), fibrocystic breast disease, fibroids of the uterus, ovarian cysts, polycystic ovary syndrome, pre-eclampsia, eclampsia of pregnancy, preterm labor, premenstrual syndrome, or vaginal dryness comprising administering a therapeutically effective amount of a compound of formulas 48-51, 53-58, 99A-99H, 120, 1072-1076, 1078-1080, and 1082-1094.

The invention encompasses methods of treating, suppressing, reducing the incidence, reducing the severity, or inhibiting the progression of a hormonal condition in a male in need thereof, comprising administering a therapeutically effective amount of a compound of formulas 48-51, 53-58, 99A-99H, 120, 1072-1076, 1078-1080, and 1082-1094, or its isomer, optical isomer, or any mixture of optical isomers, pharmaceutically acceptable salt, pharmaceutical product, polymorph, hydrate or any combination thereof. In one embodiment, the hormonal condition includes, but is not limited to, hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers. In another embodiment, the hormonal condition in a male is an androgen-dependent hormonal condition in a male.

SARDs of this invention may also find utility in treatment of sexual perversion, hypersexuality, paraphilias, androgen psychosis, virilization, androgen insensitivity syndromes (AIS) (such as complete AIS (CAIS) and partial AIS (PAIS)), and improving ovulation in an animal.

The invention encompasses methods of treating sexual perversion, hypersexuality, paraphilias, androgen psychosis, virilization androgen, insensitivity syndromes, increasing or modulating or improving ovulation comprising administering a therapeutically effective amount of a compound of formulas 48-51, 53-58, 99A-99H, 120, 1072-1076, 1078-1080, and 1082-1094.

SARDs of this invention may also be useful for treating hormone-dependent cancers such as prostate cancer, breast cancer, testicular cancer, ovarian cancer, hepatocellular carcinoma, urogenital cancer, etc. In another embodiment, the breast cancer is triple negative breast cancer. Further, local or systemic SARD administration may be useful for treatment of precursors of hormone-dependent cancers such as prostatic intraepithelial neoplasia (PIN) and atypical small acinar proliferation (ASAP).

The invention encompasses methods of treating breast cancer, testicular cancer, uterine cancer, ovarian cancer, urogenital cancer, precursors of prostate cancer, or AR related or AR expressing solid tumors, comprising administering a therapeutically effective amount of a compound of formulas 48-51, 53-58, 99A-99H, 120, 1072-1076, 1078-1080, and 1082-1094. A precursor of prostate cancers may be prostatic intraepithelial neoplasia (PIN) or atypical small acinar proliferation (ASAP). The tumor may be hepatocellular carcinoma (HCC) or bladder cancer. Serum testosterone may be positively linked to the development of HCC. Based on epidemiologic, experimental observations, and notably the fact that men have a substantially higher risk of bladder cancer than women, androgens and/or the AR may also play a role in bladder cancer initiation.

Although traditional antiandrogens such as darolutamide, enzalutamide, apalutamide, bicalutamide and flutamide and androgen deprivation therapies (ADT) such as leuprolide were approved for use in prostate cancer, there is significant evidence that antiandrogens could also be used in a variety of other hormone-dependent and hormone-independent cancers. For example, antiandrogens have been successfully tested in breast cancer (enzalutamide; Breast Cancer Res (2014) 16(1): R7), non-small cell lung cancer (shRNAi AR), renal cell carcinoma (ASC-J9), partial androgen insensitivity associated malignancies such as gonadal tumors and seminoma, advanced pancreatic cancer (World J Gastroenterology 20(29):9229), cancer of the ovary, fallopian tubes, or peritoneum, cancer of the salivary gland (Head and Neck (2016) 38: 724-731; ADT was tested in AR-expressing recurrent/metastatic salivary gland cancers and was confirmed to have benefit on progression free survival and overall survival endpoints), bladder cancer (Oncotarget 6 (30): 29860-29876); Int J Endocrinol (2015), Article ID 384860), pancreatic cancer, lymphoma (including mantle cell), and hepatocellular carcinoma. Use of a more potent antiandrogen such as a SARD in these cancers may treat the progression of these and other cancers. Other AR-expressing cancers may also benefit from SARD treatment such as testicular cancer, uterine cancer, ovarian cancer, urogenital cancer, breast cancer, brain cancer, skin cancer, lymphoma, liver cancer, renal cancer, osteosarcoma, pancreatic cancer, endometrial cancer, lung cancer, non-small cell lung cancer (NSCLC), colon cancer, perianal adenoma, or central nervous system cancer.

SARDs of this invention may also be useful for treating other cancers containing AR such as breast, brain, skin, ovarian, bladder, lymphoma, liver, kidney, pancreas, endometrium, lung (e.g., NSCLC), colon, perianal adenoma, osteosarcoma, CNS, melanoma, hypercalcemia of malignancy and metastatic bone disease, etc.

Thus, the invention encompasses methods of treating hypercalcemia of malignancy, metastatic bone disease, brain cancer, skin cancer, bladder cancer, lymphoma, liver cancer, renal cancer, osteosarcoma, pancreatic cancer, endometrial cancer, lung cancer, central nervous system cancer, gastric cancer, colon cancer, melanoma, amyotrophic lateral sclerosis (ALS), and/or uterine fibroids comprising administering a therapeutically effective amount of a compound of formulas 48-51, 53-58, 99A-99H, 120, 1072-1076, 1078-1080, and 1082-1094. The lung cancer may be non-small cell lung cancer (NSCLC).

SARDs of this invention may also be useful for the treating of non-hormone-dependent cancers. Non-hormone-dependent cancers include liver, salivary duct, etc.

In another embodiment, the SARDs of this invention are used for treating gastric cancer. In another embodiment, the SARDs of this invention are used for treating salivary duct carcinoma. In another embodiment, the SARDs of this invention are used for treating bladder cancer. In another embodiment, the SARDs of this invention are used for treating esophageal cancer. In another embodiment, the SARDs of this invention are used for treating pancreatic cancer. In another embodiment, the SARDs of this invention are used for treating colon cancer. In another embodiment, the SARDs of this invention are used for treating non-small cell lung cancer. In another embodiment, the SARDs of this invention are used for treating renal cell carcinoma.

AR plays a role in cancer initiation in hepatocellular carcinoma (HCC). Therefore, targeting AR may be an appropriate treatment for patients with early stage HCC. In late-stage HCC disease, there is evidence that metastasis is suppressed by androgens. In another embodiment, the SARDs of this invention are used for treating hepatocellular carcinoma (HCC).

Locati et al. in Head & Neck, 2016, 724-731 demonstrated the use of androgen deprivation therapy (ADT) in AR-expressing recurrent/metastatic salivary gland cancers and confirmed improved progression free survival and overall survival endpoints with ADT. In another embodiment, the SARDs of this invention are used for treating salivary gland cancer.

Kawahara et al. in Oncotarget, 2015, Vol 6 (30), 29860-29876 demonstrated that ELK1 inhibition, together with AR inactivation, has the potential of being a therapeutic approach for bladder cancer. McBeth et al. Int J Endocrinology, 2015, Vol 2015, Article ID 384860 suggested that the combination of antiandrogen therapy plus glucocorticoids as treatment of bladder cancer as this cancer is believed to have an inflammatory etiology. In another embodiment, the SARDs of this invention are used for treating bladder cancer, optionally in combination with glucocorticoids.

### Abdominal Aortic Aneurysm (AAA)

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, et al. J Vasc Surg (2016) 63(6):1602-1612) showed that flutamide (50 mg/kg) or ketoconazole (150 mg/kg) attenuated AAA induced by porcine pancreatic elastase (0.35 U/mL) by 84.2% and 91.5% compared to vehicle (121%). Further AR −/− mice showed attenuated AAA growth (64.4%) compared to wildtype (both treated with elastase). Correspondingly, administration of a SARD to a patient suffering from an AAA may help reverse, treat or delay progression of AAA to the point where surgery is needed.

### Treatment of Wounds

Wounds and/or ulcers are normally found protruding from the skin or on a mucosal surface or as a result of an infarction in an organ. A wound may be a result of a soft tissue defect or a lesion or of an underlying condition. The term “wound” denotes a bodily injury with disruption of the normal integrity of tissue structures, sore, lesion, necrosis, and/or ulcer. The term “sore” refers to any lesion of the skin or mucous membranes and the term “ulcer” refers to a local defect, or excavation, of the surface of an organ or tissue, which is produced by the sloughing of necrotic tissue. “Lesion” generally includes any tissue defect. “Necrosis” refers to dead tissue resulting from infection, injury, inflammation, or infarctions. All of these are encompassed by the term “wound,” which denotes any wound at any particular stage in the healing process including the stage before any healing has initiated or even before a specific wound like a surgical incision is made (prophylactic treatment).

Examples of wounds which can be treated in accordance with the present invention are aseptic wounds, contused wounds, incised wounds, lacerated wounds, non-penetrating wounds (i.e. wounds in which there is no disruption of the skin but there is injury to underlying structures), open wounds, penetrating wounds, perforating wounds, puncture wounds, septic wounds, subcutaneous wounds, etc. Examples of sores include, but are not limited to, bed sores, canker sores, chrome sores, cold sores, pressure sores, etc. Examples of ulcers include, but are not limited to, peptic ulcer, duodenal ulcer, gastric ulcer, gouty ulcer, diabetic ulcer, hypertensive ischemic ulcer, stasis ulcer, ulcus cruris (venous ulcer), sublingual ulcer, submucous ulcer, symptomatic ulcer, trophic ulcer, tropical ulcer, veneral ulcer, e.g., caused by gonorrhoea (including urethritis, endocervicitis and proctitis). Conditions related to wounds or sores which may be successfully treated according to the invention include, but are not limited to, burns, anthrax, tetanus, gas gangrene, scalatina, erysipelas, sycosis barbae, folliculitis, impetigo contagiosa, impetigo bullosa, etc. It is understood, that there may be an overlap between the use of the terms “wound” and “ulcer,” or “wound” and “sore” and, furthermore, the terms are often used at random.

The kinds of wounds to be treated according to the invention include also: i) general wounds such as, e.g., surgical, traumatic, infectious, ischemic, thermal, chemical and bullous wounds; ii) wounds specific for the oral cavity such as, e.g., post-extraction wounds, endodontic wounds especially in connection with treatment of cysts and abscesses, ulcers and lesions of bacterial, viral or autoimmunological origin, mechanical, chemical, thermal, infectious and lichenoid wounds; herpes ulcers, stomatitis aphthosa, acute necrotising ulcerative gingivitis and burning mouth syndrome are specific examples; and iii) wounds on the skin such as, e.g., neoplasm, burns (e.g. chemical, thermal), lesions (bacterial, viral, autoimmunological), bites and surgical incisions. Another way of classifying wounds is by tissue loss, where: i) small tissue loss (due to surgical incisions, minor abrasions, and minor bites) or ii) significant tissue loss. The latter group includes ischemic ulcers, pressure sores, fistulae, lacerations, severe bites, thermal burns and donor site wounds (in soft and hard tissues) and infarctions. Other wounds include ischemic ulcers, pressure sores, fistulae, severe bites, thermal burns, or donor site wounds.

Ischemic ulcers and pressure sores are wounds, which normally only heal very slowly and especially in such cases an improved and more rapid healing is of great importance to the patient. Furthermore, the costs involved in the treatment of patients suffering from such wounds are markedly reduced when the healing is improved and takes place more rapidly.

Donor site wounds are wounds which e.g. occur in connection with removal of hard tissue from one part of the body to another part of the body e.g. in connection with transplantation. The wounds resulting from such operations are very painful and an improved healing is therefore most valuable.

In one case, the wound to be treated is selected from the group consisting of aseptic wounds, infarctions, contused wounds, incised wounds, lacerated wounds, non-penetrating wounds, open wounds, penetrating wounds, perforating wounds, puncture wounds, septic wounds, and subcutaneous wounds.

The invention encompasses methods of treating a subject suffering from a wound comprising administering to the subject a therapeutically effective amount of a compound of formulas 48-51, 53-58, 99A-99H, 120, 1072-1076, 1078-1080, and 1082-1094, pharmaceutically acceptable salt thereof, or a pharmaceutical compositions thereof.

The invention encompasses methods of treating a subject suffering from a burn comprising administering to the subject a therapeutically effective amount of a compound of formulas 48-51, 53-58, 99A-99H, 120, 1072-1076, 1078-1080, and 1082-1094, pharmaceutically acceptable salt thereof, or a pharmaceutical composition thereof.

The term “skin” is used in a very broad sense embracing the epidermal layer of the skin and in those cases where the skin surface is more or less injured also the dermal layer of the skin. Apart from the stratum corneum, the epidermal layer of the skin is the outer (epithelial) layer and the deeper connective tissue layer of the skin is called the dermis.

Since the skin is the most exposed part of the body, it is particularly susceptible to various kinds of injuries such as, e.g., ruptures, cuts, abrasions, burns and frostbites or injuries arising from various diseases. Furthermore, much skin is often destroyed in accidents. However, due to the important barrier and physiologic function of the skin, the integrity of the skin is important to the well-being of the individual, and any breach or rupture represents a threat that must be met by the body in order to protect its continued existence.

Apart from injuries on the skin, injuries may also be present in all kinds of tissues (i.e. soft and hard tissues). Injuries on soft tissues including mucosal membranes and/or skin are especially relevant in connection with the present invention.

Healing of a wound on the skin or on a mucosal membrane undergoes a series of stages that results either in repair or regeneration of the skin or mucosal membrane. In recent years, regeneration and repair have been distinguished as the two types of healing that may occur. Regeneration may be defined as a biological process whereby the architecture and function of lost tissue are completely renewed. Repair, on the other hand, is a biological process whereby continuity of disrupted tissue is restored by new tissues which do not replicate the structure and function of the lost ones.

The majority of wounds heal through repair, meaning that the new tissue formed is structurally and chemically unlike the original tissue (scar tissue). In the early stage of the tissue repair, one process which is almost always involved is the formation of a transient connective tissue in the area of tissue injury. This process starts by formation of a new extracellular collagen matrix by fibroblasts. This new extracellular collagen matrix is then the support for a connective tissue during the final healing process. The final healing is, in most tissues, a scar formation containing connective tissue. In tissues which have regenerative properties, such as, e.g., skin and bone, the final healing includes regeneration of the original tissue. This regenerated tissue has frequently also some scar characteristics, e.g. a thickening of a healed bone fracture.

Under normal circumstances, the body provides mechanisms for healing injured skin or mucosa in order to restore the integrity of the skin barrier or the mucosa. The repair process for even minor ruptures or wounds may take a period of time extending from hours and days to weeks. However, in ulceration, the healing can be very slow and the wound may persist for an extended period of time, i.e. months or even years.

Burns are associated with reduced testosterone levels, and hypogonadism is associated with delayed wound healing. The invention encompasses methods for treating a subject suffering from a wound or a burn by administering at least one SARD compound according to this invention. The SARD may promote resolving of the burn or wound, participates in the healing process of a burn or a wound, or, treats a secondary complication of a burn or wound.

The treatment of burns or wounds may further use at least one growth factor such as epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), or any combination thereof, which promote wound healing.

Wound healing may be measured by many procedures known in the art, including, but not limited to, wound tensile strength, hydroxyproline or collagen content, procollagen expression, or re-epithelialization. As an example, a SARD as described herein may be administered orally or topically at a dosage of about 0.1-100 mg per day. Therapeutic effectiveness is measured as effectiveness in enhancing wound healing as compared to the absence of the SARD compound. Enhanced wound healing may be measured by known techniques such as decrease in healing time, increase in collagen density, increase in hydroxyproline, reduction in complications, increase in tensile strength, and increased cellularity of scar tissue.

The term “reducing the pathogenesis” is to be understood to encompass reducing tissue damage, or organ damage associated with a particular disease, disorder or condition. The term may include reducing the incidence or severity of an associated disease, disorder or condition, with that in question or reducing the number of associated diseases, disorders or conditions with the indicated, or symptoms associated thereto.

### Pharmaceutical Compositions

The compounds of the invention may be used in pharmaceutical compositions. As used herein, “pharmaceutical composition” means either the compound or pharmaceutically acceptable salt of the active ingredient with a pharmaceutically acceptable carrier or diluent. A “therapeutically effective amount” as used herein refers to that amount which provides a therapeutic effect for a given indication and administration regimen.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. The subjects may be a male or female subject or both.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The pharmaceutical compositions of the invention can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the composition can be delivered to tissue (e.g., needle or catheter). Alternatively, a topical administration may be desired for application to dermal, ocular, or mucosal surfaces. Another method of administration is via aspiration or aerosol formulation. The pharmaceutical compositions may be administered topically to body surfaces, and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

Suitable dosage forms include, but are not limited to, oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Depending on the indication, formulations suitable for oral or topical administration are preferred.

Topical Administration: The compounds of formulas 48-51, 53-58, 99A-99H, 120, 1072-1076, 1078-1080, and 1082-1094 may be administered topically. As used herein, “topical administration” refers to application of the compounds of formulas 48-51, 53-58, 99A-99H, 120, 1072-1076, 1078-1080, and 1082-1094 (and optional carrier) directly to the skin and/or hair. The topical composition can be in the form of solutions, lotions, salves, creams, ointments, liposomes, sprays, gels, foams, roller sticks, and any other formulation routinely used in dermatology.

Topical administration is used for indications found on the skin, such as hirsutism, alopecia, acne, and excess sebum. The dose will vary, but as a general guideline, the compound will be present in a dermatologically acceptable carrier in an amount of from about 0.01 to 50 w/w %, and more typically from about 0.1 to 10 w/w %. Typically, the dermatological preparation will be applied to the affected area from 1 to 4 times daily. “Dermatologically acceptable” refers to a carrier which may be applied to the skin or hair, and which will allow the drug to diffuse to the site of action. More specifically “site of action”, it refers to a site where inhibition of androgen receptor or degradation of the androgen receptor is desired.

The compounds of formulas 48-51, 53-58, 99A-99H, 120, 1072-1076, 1078-1080, and 1082-1094 may be used topically to relieve alopecia, especially androgenic alopecia. Androgens have a profound effect on both hair growth and hair loss. In most body sites, such as the beard and pubic skin, androgens stimulate hair growth by prolonging the growth phase of the hair cycle (anagen) and increasing follicle size. Hair growth on the scalp does not require androgens but, paradoxically, androgens are necessary for the balding on the scalp in genetically predisposed individuals (androgenic alopecia) where there is a progressive decline in the duration of anagen and in hair follicle size. Androgenic alopecia is also common in women where it usually presents as a diffuse hair loss rather than showing the patterning seen in men.

While the compounds of formulas 48-51, 53-58, 99A-99H, 120, 1072-1076, 1078-1080, and 1082-1094 will most typically be used to alleviate androgenic alopecia, the compounds may be used to alleviate any type of alopecia. Examples of non-androgenic alopecia include, but are not limited to, alopecia areata, alopecia due to radiotherapy or chemotherapy, scarring alopecia, or stress related alopecia.

The compounds of formulas 48-51, 53-58, 99A-99H, 120, 1072-1076, 1078-1080, and 1082-1094 can be applied topically to the scalp and hair to prevent, or treat balding. Further, the compound of formulas 48-51, 53-58, 99A-99H, 120, 1072-1076, 1078-1080, and 1082-1094 can be applied topically in order to induce or promote the growth or regrowth of hair on the scalp.

The invention also encompasses topically administering a compound of formula 48-51, 53-58, 99A-99H, 120, 1072-1076, 1078-1080, and 1082-1094 to treat or prevent the growth of hair in areas where such hair growth in not desired. One such use will be to alleviate hirsutism. Hirsutism is excessive hair growth in areas that typically do not have hair (e.g., a female face). Such inappropriate hair growth occurs most commonly in women and is frequently seen at menopause. The topical administration of the compounds of formulas 48-51, 53-58, 99A-99H, 120, 1072-1076, 1078-1080, and 1082-1094 will alleviate this condition leading to a reduction, or elimination of this inappropriate, or undesired, hair growth.

The compounds of formulas 48-51, 53-58, 99A-99H, 120, 1072-1076, 1078-1080, and 1082-1094 may also be used topically to decrease sebum production. Sebum is composed of triglycerides, wax esters, fatty acids, sterol esters and squalene. Sebum is produced in the acinar cells of the sebaceous glands and accumulates as these cells age. At maturation, the acinar cells lyse, releasing sebum into the luminal duct so that it may be deposited on the surface of the skin.

In some individuals, an excessive quantity of sebum is secreted onto the skin. This can have a number of adverse consequences. It can exacerbate acne, since sebum is the primary food source for Propionbacterium acnes, the causative agent of acne. It can cause the skin to have a greasy appearance, typically considered cosmetically unappealing.

Formation of sebum is regulated by growth factors and a variety of hormones including androgens. The cellular and molecular mechanism by which androgens exert their influence on the sebaceous gland has not been fully elucidated. However, clinical experience documents the impact androgens have on sebum production. Sebum production is significantly increased during puberty, when androgen levels are their highest. The compounds of formulas 48-51, 53-58, 99A-99H, 120, 1072-1076, 1078-1080, and 1082-1094 inhibit the secretion of sebum and thus reduce the amount of sebum on the surface of the skin. The compounds of formulas 48-51, 53-58, 99A-99H, 120, 1072-1076, 1078-1080, and 1082-1094 can be used to treat a variety of dermal diseases such as acne or seborrheic dermatitis.

In addition to treating diseases associated with excess sebum production, the compounds of formulas 48-51, 53-58, 99A-99H, 120, 1072-1076, 1078-1080, and 1082-1094 can also be used to achieve a cosmetic effect. Some consumers believe that they are afflicted with overactive sebaceous glands. They feel that their skin is oily and thus unattractive. These individuals may use the compounds of formulas 48-51, 53-58, 99A-99H, 120, 1072-1076, 1078-1080, and 1082-1094 to decrease the amount of sebum on their skin. Decreasing the secretion of sebum will alleviate oily skin in individuals afflicted with such conditions.

To treat these topical indications, the invention encompasses cosmetic or pharmaceutical compositions (such as dermatological compositions), comprising at least one of the compounds of formulas 48-51, 53-58, 99A-99H, 120, 1072-1076, 1078-1080, and 1082-1094. Such dermatological compositions will contain from 0.001% to 10% w/w % of the compound(s) in admixture with a dermatologically acceptable carrier, and more typically, from 0.1 to 5 w/w % of the compounds. Such compositions will typically be applied from 1 to 4 times daily. The reader's attention is directed to Remington's Pharmaceutical Science, Edition 17, Mark Publishing Co., Easton, Pa. for a discussion of how to prepare such formulations.

The compositions of the invention may also include solid preparations such as cleansing soaps or bars. These compositions are prepared according to methods known in the art.

Formulations such as aqueous, alcoholic, or aqueous-alcoholic solutions, or creams, gels, emulsions or mousses, or aerosol compositions with a propellant may be used to treat indications that arise where hair is present. Thus, the composition can also be a hair care composition. Such hair care compositions include, but are not limited to, shampoo, a hair-setting lotion, a treating lotion, a styling cream or gel, a dye composition, or a lotion or gel for preventing hair loss. The amounts of the various constituents in the dermatological compositions are those conventionally used in the fields considered.

Medicinal and cosmetic agents containing the compounds of formulas 48-51, 53-58, 99A-99H, 120, 1072-1076, 1078-1080, and 1082-1094 will typically be packaged for retail distribution (i.e., an article of manufacture). Such articles will be labeled and packaged in a manner to instruct the patient how to use the product. Such instructions will include the condition to be treated, duration of treatment, dosing schedule, etc.

Antiandrogens, such as finasteride or flutamide, have been shown to decrease androgen levels or block androgen action in the skin to some extent but suffer from undesirable systemic effects. An alternative approach is to topically apply a selective androgen receptor degrader (SARD) compound to the affected areas. Such SARD compound would exhibit potent but local inhibition of AR activity, and local degradation of the AR, would not penetrate to the systemic circulation of the subject, or would be rapidly metabolized upon entry into the blood, limiting systemic exposure.

To prepare such pharmaceutical dosage forms, the active ingredient may be mixed with a pharmaceutical carrier according to conventional pharmaceutical compounding techniques. The carrier may take a wide variety of forms depending on the form of preparation desired for administration.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Oral and Parenteral Administration: In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients may be included, such as ingredients that aid solubility or for preservation. Injectable solutions may also be prepared in which case appropriate stabilizing agents may be employed.

In some applications, it may be advantageous to utilize the active agent in a ^{“}vectorized^{”} form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

For administration to mammals, and particularly humans, it is expected that the physician will determine the actual dosage and duration of treatment, which will be most suitable for an individual and can vary with the age, weight, genetics and/or response of the particular individual.

The methods of the invention comprise administration of a compound at a therapeutically effective amount. The therapeutically effective amount may include various dosages.

In one embodiment, a compound of this invention is administered at a dosage of 1-3000 mg per day. In additional embodiments, a compound of this invention is administered at a dose of 1-10 mg per day, 3-26 mg per day, 3-60 mg per day, 3-16 mg per day, 3-30 mg per day, 10-26 mg per day, 15-60 mg, 50-100 mg per day, 50-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day. In one embodiment, a compound of this invention is administered at a dosage of 25 mg per day. In one embodiment, a compound of this invention is administered at a dosage of 40 mg per day. In one embodiment, a compound of this invention is administered at a dosage of 50 mg per day. In one embodiment, a compound of this invention is administered at a dosage of 67.5 mg per day. In one embodiment, a compound of this invention is administered at a dosage of 75 mg per day. In one embodiment, a compound of this invention is administered at a dosage of 80 mg per day. In one embodiment, a compound of this invention is administered at a dosage of 100 mg per day. In one embodiment, a compound of this invention is administered at a dosage of 125 mg per day. In one embodiment, a compound of this invention is administered at a dosage of 250 mg per day. In one embodiment, a compound of this invention is administered at a dosage of 300 mg per day. In one embodiment, a compound of this invention is administered at a dosage of 500 mg per day. In one embodiment, a compound of this invention is administered at a dosage of 600 mg per day. In one embodiment, a compound of this invention is administered at a dosage of 1000 mg per day. In one embodiment, a compound of this invention is administered at a dosage of 1500 mg per day. In one embodiment, a compound of this invention is administered at a dosage of 2000 mg per day. In one embodiment, a compound of this invention is administered at a dosage of 2500 mg per day. In one embodiment, a compound of this invention is administered at a dosage of 3000 mg per day.

The methods may comprise administering a compound at various dosages. For example, the compound may be administered at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

The pharmaceutical composition may be a solid dosage form, a solution, or a transdermal patch. Solid dosage forms include, but are not limited to, tablets and capsules.

The following examples are presented in order to more fully illustrate the preferred embodiments of the invention. They should in no way, however, be construed as limiting the broad scope of the invention.

### EXAMPLES

### Example 1: Synthesis of SARDs

### (S)-N-(4-Cyano-3-(trifluoromethyl)phenyl)-3-(4-ethynyl-1H-pyrazol-1-yl)-2-hydroxy-2-methylpropanamide (C₁₇H₁₃F₃N₄O₂) (1072)

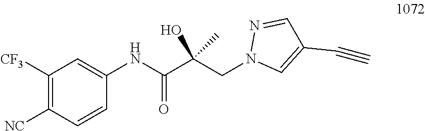 [SMILES: C#Cc1cnn(C[C@](C)(O)C(=O)Nc2ccc(C#N)c(C(F)(F)F)c2)c1]

To a solution of 4-ethynyl-1H-pyrazole (0.15 g, 0.001629 mol) in anhydrous THF (10 mL), which was cooled in an ice water bath under an argon atmosphere, was added sodium hydride (60% dispersion in oil, 0.33 g, 0.0081433 mol). After addition, the resulting mixture was stirred for 3 h. (R)-3-Bromo-N-(4-cyano-3-(trifluoromethyl)phenyl)-2-hydroxy-2-methylpropanamide (0.57 g, 0.001629 mol) was added to above solution, and the resulting reaction mixture was allowed to stir overnight at room temperature (RT) under argon. The reaction was quenched by water, and extracted with ethyl acetate. The organic layer was washed with brine, dried with MgSO₄, filtered, and concentrated under vacuum. The product was purified by a silica gel column using DCM and ethyl acetate (95:5) as eluent to afford 0.37 g (62.7%) of the titled compound as white foam.

¹H NMR (400 MHz, DMSO-d₆) δ 10.40 (s, 1H, NH), 8.47 (s, 1H, ArH), 8.24 (d, J=8.8 Hz, 1H, ArH), 8.11 (d, J=8.8 Hz, 1H, ArH), 7.91 (s 1H, Pyrazole-H), 7.57 (s 1H, Pyrazole-H), 6.35 (s, 1H, OH), 4.46 (d, J=14.4 Hz, 1H, CH), 4.29 (d, J=14.4 Hz, 1H, CH), 4.00 (s, 1H, CH), 1.35 (s, 3H, CH3). HRMS [C17H14F3N4O2⁻]:calcd 363.1069, found 363.1026 [M+H]⁺. Purity: 99.55% (HPLC).

### (S)-4-(5((4-Fluoro-1H-pyrazol-1-yl)methyl)-5-methyl-2,4-dioxooxazolidin-3-yl)-2-(trifluoromethyl)benzonitrile (C₁₆H₁₀F₄N₄O₃) (1073)

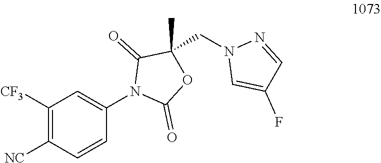 [SMILES: C[C@@]1(Cn2cc(F)cn2)OC(=O)N(c2ccc(C#N)c(C(F)(F)F)c2)C1=O]

To a solution of (S)-N-(4-cyano-3-(trifluoromethyl)phenyl)-3-(4-fluoro-1H-pyrazol-1-yl)-2-hydroxy-2-methylpropanamide (0.234 g, 0.0006568 mol) in anhydrous pyridine (8 mL) was added 1,1′-carbonyldiimidazole (CDI) (0.16 g, 0.0009825 mol). After addition, the resulting mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (2:1) as eluent to afford 0.134 g (42%) of the titled compound as white foam.

¹H NMR (400 MHz, DMSO-d₆) δ 8.41 (d, J=8.0 Hz, 1H, ArH), 7.98 (s, 1H, ArH), 7.94 (d, J=4.0 Hz, 1H, Pyrazole-H), 7.85 (d, J=8.2 Hz, 1H, ArH), 7.58 (d, J=4.4 Hz, 1H, Pyrazole-H), 4.78 (d, J=14.8 Hz, 1H, CH), 4.69 (d, J=14.8 Hz, 1H, CH), 1.71 (s, 3H, CH₃). HRMS [C₁₆H₁₁F₄N₄O₃⁺]: calcd 383.0767, found 383.0726 [M+H]⁺. Purity: 97.64% (HPLC).

### (S)-N-(4-Cyano-3-(trifluoromethyl)phenyl)-3-(4-(4-((S)-3-((4-cyano-3-(trifluoromethyl)phenyl)amino)-2-hydroxy-2-methyl-3-oxopropoxy)but-l-yn-1-yl)-1H-pyrazol-1-yl)-2-hydroxy-2-methylpropanamide (C31H26F6N605) (1074)

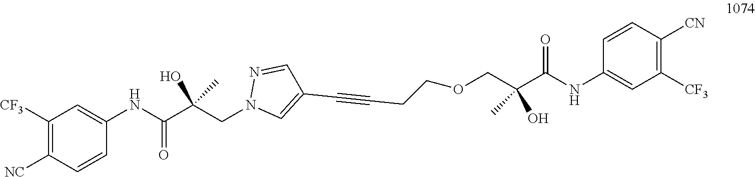 [SMILES: C[C@](O)(COCCC#Cc1cnn(C[C@](C)(O)C(=O)Nc2ccc(C#N)c(C(F)(F)F)c2)c1)C(=O)Nc1ccc(C#N)c(C(F)(F)F)c1]

To a solution of 4-(1H-pyrazol-4-yl)but-3-yn-1-ol (0.48 g, 0.0035256 mol) in anhydrous THF (20 mL), which was cooled in an ice water bath under an argon atmosphere, was added sodium hydride (60% dispersion in oil, 0.71 g, 0.0172676 mol). After addition, the resulting mixture was stirred for 3 h. (R)-3-Bromo-N-(4-cyano-3-(trifluoromethyl)phenyl)-2-hydroxy-2-methylpropanamide (1.24 g, 0.0035256 mol) was added to the above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and methanol (95:5) as eluent to afford 0.477 g (20%) of the titled compound as yellowish solid.

¹H NMR (400 MHz, DMSO-d₆) δ 10.48 (s, 1H, NH), 10.39 (s, 1H, NH), 8.54 (s, 1H, ArH), 8.46 (s, 1H, ArH), 8.29 (d, J=8.4 Hz, 1H, ArH), 8.23 (d, J=8.4 Hz, 1H, ArH), 8.12-8.06 (m, 2H, ArH), 7.73 (s 1H, Pyrazole-H), 7.39 (s 1H, Pyrazole-H), 6.31 (s, 1H, OH), 5.99 (s, 1H, OH), 4.43 (d, J=14.0 Hz, 1H, CH), 4.26 (d, J=14.0 Hz, 1H, CH), 3.71 (d, J=9.2 Hz, 1H, CH), 3.62-3.53 (m, 2H, CH₂), 4.49 (d, J=9.6 Hz, 1H, CH), 2.58-2.54 (m, 2H, CH₂), 1.36 (s, 3H, CH₃), 1.31 (s, 3H, CH₃). HRMS [C₃₁H₂6F₆N₆NaO₅⁺]: calcd 699.1767, found 699.1871 [M+Na]⁺. Purity: % (HPLC).

### (S)-N-(4-Cyano-3-(trifluoromethyl)phenyl)-2-hydroxy-3-(4-(4-hydroxybut-1-yn-1-yl)-1H-pyrazol-1-yl)-2-methylpropanamide (C₁₉H₁₇F₃N₄O₃) (1075)

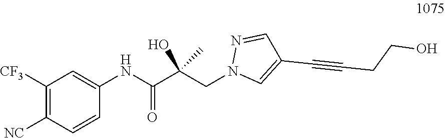 [SMILES: C[C@](O)(Cn1cc(C#CCCO)cn1)C(=O)Nc1ccc(C#N)c(C(F)(F)F)c1]

To a solution of 4-(1H-pyrazol-4-yl)but-3-yn-1-ol (0.48 g, 0.0035256 mol) in anhydrous THF (20 mL), which was cooled in an ice water bath under an argon atmosphere, was added sodium hydride (60% dispersion in oil, 0.71 g, 0.0172676 mol). After addition, the resulting mixture was stirred for 3 h. (R)-3-Bromo-N-(4-cyano-3-(trifluoromethyl)phenyl)-2-hydroxy-2-methylpropanamide (1.24 g, 0.0035256 mol) was added to above solution, and the resulting reaction mixture was allowed to stir overnight at RT under argon. The reaction was quenched by water, and extracted with ethyl acetate. The organic layer was washed with brine, dried with MgSO₄, filtered, and concentrated under vacuum. The product was purified by a silica gel column using DCM and methanol (95:5) as eluent to afford 0.477 g (20%) of the titled compound as yellowish solid.

¹H NMR (400 MHz, DMSO-d₆) δ 12.99 (brs, 1H), 10.47 (s, 1H, NH), 8.55 (s, 1H, ArH), 8.29 (d, J=8.8 Hz, 1H, ArH), 8.08 (d, J=8.8 Hz, 1H, ArH), 7.87 (s 1H, Pyrazole-H), 7.49 (s 1H, Pyrazole-H), 6.00 (s, 1H, OH), 3.64 (d, J=8.2 Hz, 1H, CH), 3.60-3.56 (m, 2H, CH₂), 4.50 (d, J=9.6 Hz, 1H, CH), 2.59-2.55 (m, 2H, CH₂), 1.31 (s, 3H, CH₃). HRMS [C₁₉H₁₈F₃N₄O₃⁺]: calcd 407.1331, found 407.1267 [M+H]⁺. HRMS [C₁₉H₁₇F₃N₄NaO₃⁺]: calcd 429.1150, found 429.1099 [M+Na]⁺. Purity: % (HPLC).

### (S)-N-(3-((4-Cyano-3-(trifluoromethyl)phenyl)amino)-2-hydroxy-2-methyl oxopropyl)-1H-pyrazole-4-carboxamide (C₁₆H₁₄F₃N₅O₃) (1076)

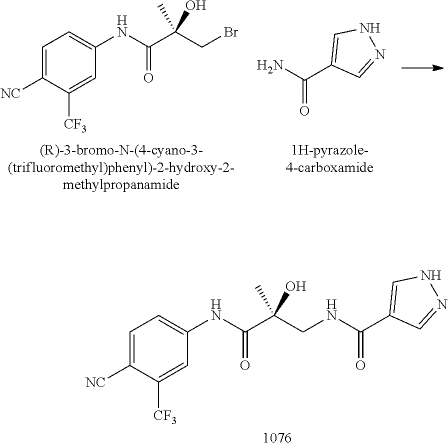 [SMILES: C[C@](O)(CBr)C(=O)Nc1ccc(C#N)c(C(F)(F)F)c1 ; NC(=O)c1cn[nH]c1 ; C[C@](O)(CNC(=O)c1cn[nH]c1)C(=O)Nc1ccc(C#N)c(C(F)(F)F)c1]

To a dry, nitrogen-purged 50 mL round-bottom flask equipped with a dropping funnel under argon atmosphere, NaH of 60% dispersion in mineral oil (165 mg, 4.1 mmol) was added in 10 mL of anhydrous THF solvent in the flask at ice-water bath, and 1H-pyrazole-4-carboxamide (227 mg, 2.05 mmol) was stirred 30 min at the ice-water bath. Into the flask, (R)-3 -bromo-N-(4-cyano-3 -(trifluoromethyl)phenyl)-2-hydroxy-2-methylpropanamide (721 mg, 2.05 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=1/1 to produce the designed compound as brown solid. The structure of product was confirmed with 2D NMR (COSY and NOESY).

Yield 43%; Brown solid; MS (ESI) m/z 380.1 [M−H]−; 382.1 [M+H]⁺; HRMS (ESI) m/z calcd for C₁₆H₁₄F₃N₅O₃382.1127 [M+H]₊ found 382.1051 [M+H]⁺; 404.0882 [M+Na]⁺; ¹H NMR (Acetone-d₆, 400 MHz) δ 9.92 (bs, 1H, NHCO), 8.44 (d, J=1.8 Hz, 1H), 8.24 (dd, J=8.8, 1.8 Hz, 1H), 8.12 (s, 1H), 8.03 (d, J=1.8 Hz, 1H), 7.84 (s, 1H), 7.11 (bs, 1H, NHCO), 6.38 (bs, 1H, NH), 5.74 (s, OH), 4.67 (d, J=14.0 Hz, 1H), 4.39 (d, J=14.0 Hz, 1H), 1.50 (s, 3H). ¹⁹F NMR (Acetone-d₆, 400 MHz) δ 114.69.

### N-(1-((S)-3-((4-Cyano-3-(trifluoromethyl)phenyl)amino)-2-hydroxy-2-methyl-3-oxopropyl)-1H-pyrazol-4-yl)-5-((4R)-2-oxohexahydro-1H-thieno[3,4-d]imidazol-4-yl)pentanamide (C₂₅H₂₈F₃N₇O₄S) (1078)

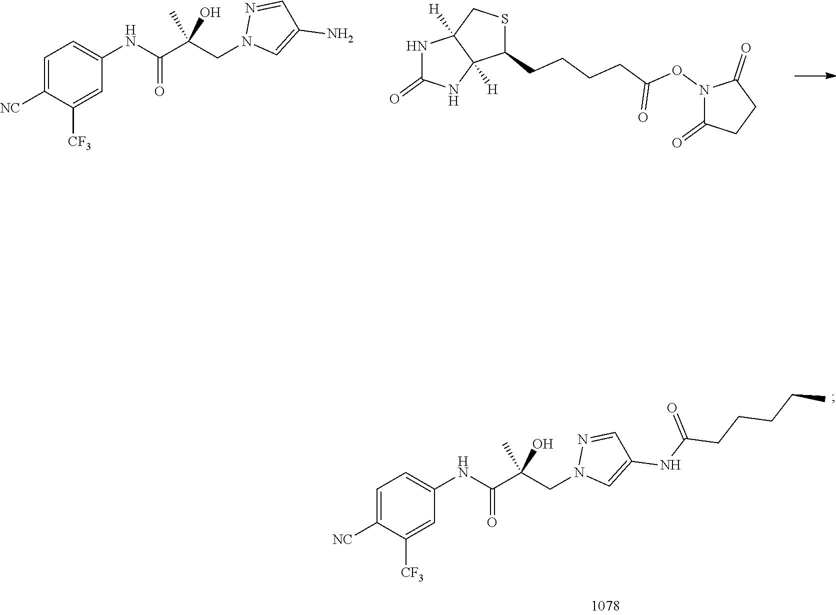

2,5-Dioxopyrrolidin-l-yl 5-((4S)-2-oxohexahydro-1H-thieno [3,4-d]imidazol-4-yl)pentanoate (Biotin-N-hydroxysuccinylimide ester, 146 mg, 0.413 mmol) was dissolved in 1 mL of DMF. (S)-3-(4-Amino-1H-pyrazol-1-yl)-N-(4-cyano-3-(trifluoromethyl)phenyl)-2-hydroxy-2-methylpropanamide (141 mg, 0.413 mmol) was added and the mixture was stirred for 30 minutes while monitored by TLC (EtOAc, 100%). After completion of reaction the solvent is removed under reduced pressure and the product is purified by column chromatography to afford the corresponding product.

Yield 48%. ¹H NMR (400 MHz, DMSO-d₆) δ =10.41 (bs, 1H, NH), 9.87 (bs, 1H, NH), 8.47 (s, 1H), 8.20 (d, J=8.4 Hz, 1H), 8.10 (d, J=8.4 Hz, 1H), 7.90 (s, 1H), 7.33 (s, 1H), 6.45 (s, 1H), 6.39 (s, 1H), 6.31 (bs, 1H, OH), 4.39 (d, J=14.0 Hz, 1H), 4.32-4.29 (m, 1H), 4.22 (d, J=14.0 Hz, 1H), 4.14-4.12 (m, 1H), 3.17 (m, 1H), 2.84 (dd, J=12.8, 5.2 Hz, 1H), 2.57 (d, J=12.8 Hz, 1H), 2.21 (t, J=7.6 Hz, 2H), 1.60-1.55 (m, 2H), 1.32 (s, 3H), 1.18 (t, J=7.2 Hz, 1H), 1.15 (t, J=7.2 Hz, 1H) HRMS (ESI) m/z calcd for C₂₅H₂₈F₃N₇O₄S 580.1954 [M+H]⁺ found 580.1973; 602.1773 [M+Na]^{+ found} 602.1808.

### tert-Butyl ((1H-pyrazol-4-yl)methyl)carbamate (C9H15N302) (1079a; intermediate)

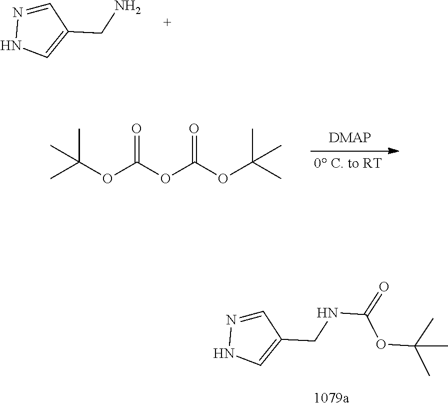

To a flask containing the compound (1H-pyrazol-4-yl)methanamine dihydrochloride (1 g, 5.89 mmol) and dichloromethane (50 mL) (treated with trimethylamine (1.64 mL, 11.76 mmol)) was added di-tert-butyldicarbonate (1.28 g, 5.89 mmol). The reaction was stirred overnight, stripped of solvent and finally purified by silica gel chromatography (EtOAc/hexane) to yield target compound as a colorless solid.

MS (ESI) m/z 198.10 [M+H]⁺; ¹H NMR (CDCl₃, 400 MHz) δ 7.48 (s, 2H), 6.68 (bs, 1H, NH), 4.72 (bs, 1H, NHC(O)—, 4.15 (d, J=4.8 Hz, 1H), 1.38 (s, 9H).

### (S)-tert-Butyl ((1-(3-((6-cyano-5-(trifluoromethyl)pyridin-3-yl)amino)-2-hydroxy-2-methyl-3-oxopropyl)-1H-pyrazol-4-yl)methyl)carbamate (C₂₀H₂₃F₃N₆O₄) (1079)

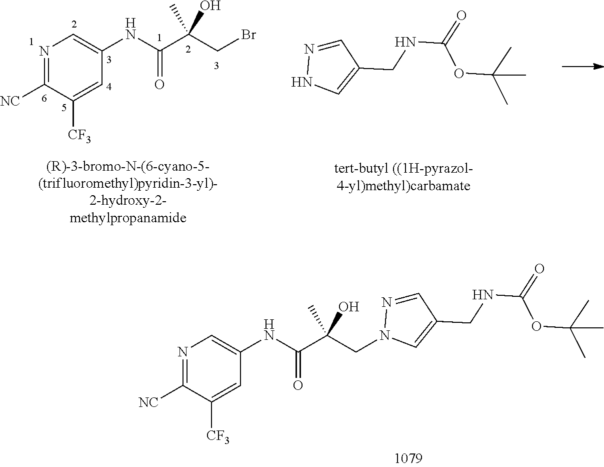 [SMILES: C[C@](O)(CBr)C(=O)Nc1cnc(C#N)c(C(F)(F)F)c1 ; CC(C)(C)OC(=O)NCc1cn[nH]c1 ; CC(C)(C)OC(=O)NCc1cnn(C[C@](C)(O)C(=O)Nc2cnc(C#N)c(C(F)(F)F)c2)c1]

To a dry, nitrogen-purged 50 mL round-bottom flask equipped with a dropping funnel under argon atmosphere, NaH of 60% dispersion in mineral oil (240 mg, 6.0 mmol) was added in 10 mL of anhydrous THF solvent in the flask at ice-water bath, and (tert-butyl ((1H-pyrazol-4-yl)methyl)carbamate (139, 396.5 mg, 2.0 mmol) was stirred 30 min at the ice-water bath. Into the flask, (R)-3-bromo-N-(6-cyano-5-(trifluoromethyl)pyridin-3-yl)-2-hydroxy-2-methylpropanamide (708 mg, 2.0 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/ hexane=1/1 to produce the designed compound as brown solid (1.043 g, Yield 81%). MP 172.5-173.6° C.

The structure of product was confirmed with 2D NMR (COSY and NOESY). MS (ESI) m/z 467.25 [M−H]⁻, 492.17 [M+Na]⁺; HRMS (ESI) m/z calcd for C₂₀H₂₃F₃N₆O₄443.0079 [M+H]⁺ found 443.0083 [M+H]⁺; 464.9903 [M+Na]⁺; ¹H NMR (CDCl₃, 400 MHz) δ 9.35 (bs, 1H, NH), 8.84 (d, J=2.0 Hz, 1H), 8.69 (d, J=2.0 Hz, 1H), 7.49 (s, 1H), 7.41 (s, 1H), 4.74 (bs, NHC(O)), 4.62 (d, J=13.6 Hz, 1H), 4.23 (d, J=13.6 Hz, 1H), 4.15 (m, 2H), 4.14 (bs, 1H, OH), 1.49 (s, 3H), 1.45 (s, 9H). ¹⁹F NMR (CDCl₃, 400 MHz) δ −62.10.

### (S)-3-(4-(Aminomethyl)-1H-pyrazol-1-yl)-N-(6-cyano-5-(trifluoromethyl)pyridin-3-yl)-2-hydroxy-2-methylpropanamide (C₁₅H₁₅F₃N₆O₂) (1080)

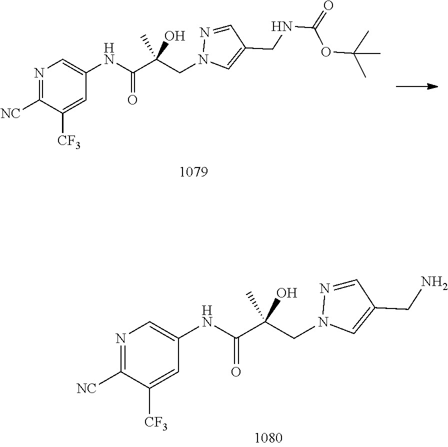 [SMILES: CC(C)(C)OC(=O)NCc1cnn(C[C@](C)(O)C(=O)Nc2cnc(C#N)c(C(F)(F)F)c2)c1 ; C[C@](O)(Cn1cc(CN)cn1)C(=O)Nc1cnc(C#N)c(C(F)(F)F)c1]

To a solution of 1079 (721 mg, 2.05 mmol) in EtOH (20 mL) was added dropwise acetyl chloride (5 mL) at 0° C. and further stirred at RT for 3 h. After removing solvent under reduced pressure the resulting solid was recrystallized from EtOAc-hexane to give the desired compound (94%) as a brown solid.

MS (ESI) m/z 367.20 [M−H]⁻; 369.24 [M+H]⁺; ¹H NMR (MeOD-d₄, 400 MHz) δ 10.13 (bs, 1H, NHCO), 9.13 (d, J=2.0 Hz, 1H), 8.78 (d, J=2.0 Hz, 1H), 8.29 (bs, 2H, NH₂), 7.92 (s, 1H), 7.61 (s, 1H), 4.65 (d, J=14.0 Hz, 1H), 4.37 (d, J=14.0 Hz, 1H), 4.21 (s, 2H), 4.04 (bs, 1H, OH), 1.53 (s, 3H). ¹⁹F NMR (MeOD-d₄, 400 MHz) δ −63.69.

### N-((1-(S)-3-(6-Cyano-5-(trifluoromethyl)pyridin-3-yl)amino)-2-hydroxy-2-methyl-3-oxopropyl)-1H-pyrazol-4-yl)methyl)-5-(4R)-2-oxohexahydro-1H-thieno [3,4-d]imidazol-4-yl)pentanamide (C₂₅H₂₉F₃N₈O₄S) (1082)

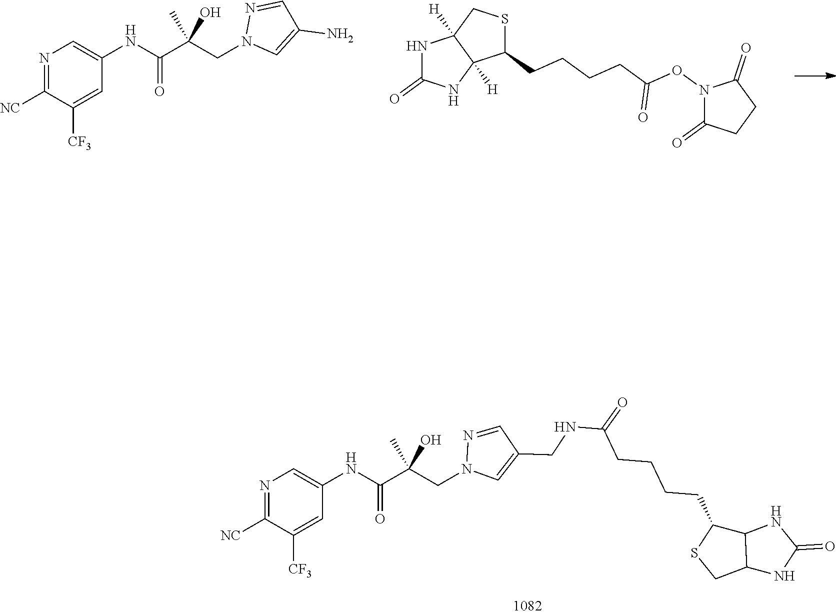 [SMILES: C[C@](O)(Cn1cc(N)cn1)C(=O)Nc1cnc(C#N)c(C(F)(F)F)c1 ; O=C1N[C@H]2[C@H](CS[C@H]2CCCCC(=O)ON2C(=O)CCC2=O)N1 ; C[C@](O)(Cn1cc(CNC(=O)CCCC[C@H]2SCC3NC(=O)NC32)cn1)C(=O)Nc1cnc(C#N)c(C(F)(F)F)c1]

2,5-Dioxopyrrolidin-1-yl 5-((4R)-2-oxohexahydro-1H-thieno [3,4-d]imidazol-4-yl)pentanoate (Biotin-NHS, 93 mg, 0.27 mmol) was dissolved in 1 mL of DMF.1080 (100 mg, 0.27 mmol) was added and the mixture is stirred overnight at RT while monitored by TLC. After completion of reaction the solvent was removed under reduced pressure and the product was purified by column chromatography (EtOAc/hexane/MeOH=4/2/1, v/v/v) to afford the desired amide (yield 38%) as white solid.

MS (ESI) m/z 593.41 [M−H]⁻; 617.23 [M+Na]⁺; ¹H NMR (MeOD-d₄, 400 MHz) δ 9.11 (s, 1H), 8.77 (s, 1H), 8.31 (bs, 1H, NHCO), 7.64 (s, 1H), 7.36 (s, 1H), 5.52 (bs, 2H), 4.53 (d, J=14.0 Hz, 1H), 4.52 (m, 1H), 4.32 (m, 1H), 4.28 (d, J=14.0 Hz, 1H), 4.18 (d, J=4.8 Hz, 2H, CH₂NH(CO)—), 3.22 (m, 1H), 2.96 (dd, J=12.8, 4.8 Hz, 1H), 2,72 (d, J=12.8 Hz, 1H), 2.20 (t, J=7.2 Hz, 2H), 1.66 (m, 2H), 1.49 (s, 3H), 1.42 (m, 2H). ¹⁹F NMR (MeOD-d₄, 400 MHz) δ −63.65.

### (S)-tert-Butyl (1-(3((4-cyano-3-(trifluoromethyl)phenyl)amino)-2-hydroxy-2-methyl-3-oxopropyl)-1H-pyrazole-4-carbonyl)carbamate (C₂₁H₂₂F₃N₅O₅) (1083a; intermediate)

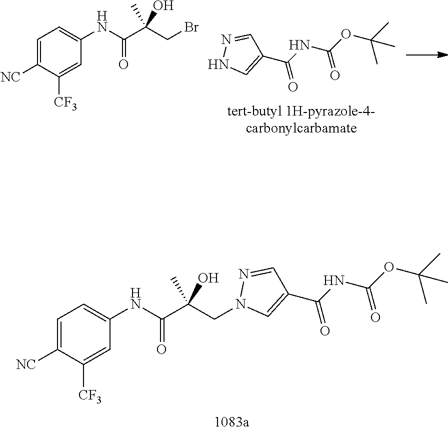 [SMILES: C[C@](O)(CBr)C(=O)Nc1ccc(C#N)c(C(F)(F)F)c1 ; CC(C)(C)OC(=O)NC(=O)c1cn[nH]c1 ; CC(C)(C)OC(=O)NC(=O)c1cnn(C[C@](C)(O)C(=O)Nc2ccc(C#N)c(C(F)(F)F)c2)c1]

To a dry, nitrogen-purged 50 mL round-bottom flask equipped with a dropping funnel under argon atmosphere, NaH of 60% dispersion in mineral oil (120 mg, 3.0 mmol) was added in 10 mL of anhydrous THF solvent in the flask at ice-water bath, and tert-butyl 1H-pyrazole-4-carbonylcarbamate (211 mg, 1.0 mmol) was stirred 30 min at the ice-water bath. Into the flask, (R)-3-bromo-N-(4-cyano-3-(trifluoromethyl)phenyl)-2-hydroxy-2-methylpropanamide (351 mg, 1.0 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/ hexane=3/2 to produce the designed compound as white solid (0.333 mg, Yield 69%).

The structure of product was confirmed with 2D NMR (COSY and NOESY). MS (ESI) m/z 480.23 [M−H]⁻; HRMS (ESI) m/z calcd for C₂₁H₂₂F₃N₅O₅382.1127 [(M-t-Boc)+H] ⁺ found 382.1129 [(M-t-Boc)+H]⁺; ¹H NMR (CDCl₃, 400 MHz) δ 9.13 (bs, 1H, NH), 8.18 (d, J=10.8 Hz, 1H), 8.02 (d, J=1.2 Hz, 1H), 7.94 (s, 1H), 7.89 (d, J=8.4 Hz, 1H), 7.77 (d, J=8.4 Hz, 1H), 7.66 (bs, C(O)NHC(O)), 5.79 (bs, 1H, OH), 4.70 (d, J=13.8 Hz, 1H), 4.32 (d, J=13.8 Hz, 1H), 1.52 (s, 3H), 1.50 (s, 9H); ¹⁹F NMR (CDCl₃, 400 MHz) δ −62.20.

### (S)-1-(3((4-Cyano-3-(trifluoromethyl)phenyl)amino)-2-hydroxy-2-methyl-3-oxopropyl)-1H-pyrazole-4-carboxamide (C₁₆H₁₄F₃N₅O₃) (1083)

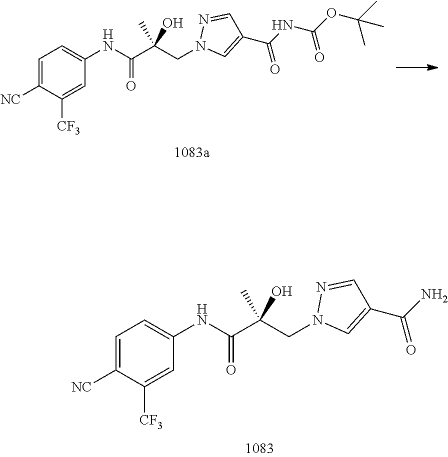 [SMILES: CC(C)(C)OC(=O)NC(=O)c1cnn(C[C@](C)(O)C(=O)Nc2ccc(C#N)c(C(F)(F)F)c2)c1 ; C[C@](O)(Cn1cc(C(N)=O)cn1)C(=O)Nc1ccc(C#N)c(C(F)(F)F)c1]

To a solution of 1083a (721 mg, 2.05 mmol) in EtOH (10 mL) was added dropwise acetyl chloride (3 mL) at 0° C. and further stirred at RT for 3 h. After removing solvent under reduced pressure, the mixture was treated with ethylacetate and hexane to give the desired compound (95%) as a yellowish solid.

Purity: 98.75%; the structure of product was confirmed with 2D NMR (COSY and NOESY). UV max 194.45, 270.45. MS (ESI) m/z 380.19 [M−H]⁻; HRMS (ESI) m/z calcd for C₁₆H₁₄F₃N₅O₃382.1127 [M+H]⁺, found 382.1282 [M+H]⁺; ¹H NMR (DMSO-d₆, 400 MHz) δ 10.39 (bs, 1H, NHC(O )), 8.46 (d, J=1.6 Hz, 1H), 8.20 (dd, J=8.6, 1.6 Hz, 1H), 8.08 (d, J=8.6 Hz, 1H), 8.05 (s, 1H), 7.75 (s, 1H), 7.55 (bs, 2H, C(O)NH₂), 6.99 (bs, 1H, OH), 4.45 (d, J=13.8 Hz, 1H), 4.28 (d, J=13.8 Hz, 1H), 1.34 (s, 3H). ¹⁹F NMR DMSO-d₆, 400 MHz) δ −61.13.

### Ethyl-5-fluoro-1H-indole-3-carboxylate (C₁₁H₁₀FNO₂) (48a; intermediate)

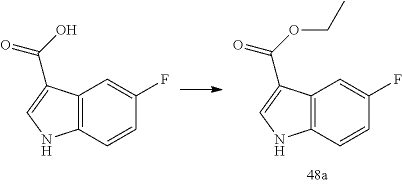

To 30 mL of ethanol, (1.0 mol) of carboxylic acid (1.5 g, 8.37 mmol) was added in a 100-mL round-bottomed flask. To this, a catalytic amount of concentrated H₂SO₄ was added at RT and was heated under reflux with continuous stirring overnight. The reaction was monitored by TLC using ethyl acetate and hexane (2:3, v/v) system. Stirring was continued until TLC indicated the completion of reaction. Then the reaction mixture was allowed to reach the RT. Ethanol was distilled off under reduced pressure and the residue was dissolved in water and then extracted twice with ethyl acetate. The combined organic solutions were washed with saturated NaHCO3 solution, water and dried over anhydrous MgSO4. The solvent was removed under vacuum to get the target compound as a light brown solid (75%).

HRMS (ESI) m/z calcd for C₁₁H₁₀FNO₂208.0774 [M+H]⁺ found 208.0817 [M+H]⁺; MS (ESI) m/z 208.10 [M+H]⁺; ¹H NMR (400 MHz, CDCl₃) δ 8.62 (bs, 1H, NH), 7.99 (d, J=2.8 Hz, 1H), 7.86 (d, J=10.0, 2.8 Hz, 1H), 7.37 (dd, J=8.8, 4.4 Hz, 1H), 7.05 (dt, J=8.8, 1.4 Hz, 1H), 4.43 (q, J=6.8 Hz, 1H), 1.45 (t, J=7.2 Hz, 1H). ¹⁹F NMR (400 MHz, CDCl₃) δ −121.38.

### (S)-Ethyl 1-(3-(4-cyano-3-(trifluoromethyl)phenyl)amino)-2-hydroxy-2-methyl-3-oxopropyl)-5-fluoro-1H-indole-3-carboxylate (C₂₃H₁₉F₄N₃O₄) (48)

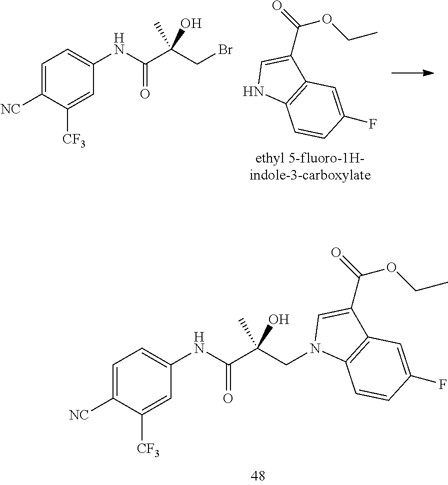

To a dry, nitrogen-purged 50 mL round-bottom flask equipped with a dropping funnel under argon atmosphere, NaH of 60% dispersion in mineral oil (116 mg, 2.9 mmol) was added in 10 mL of anhydrous THF solvent in the flask at ice-water bath, and a solution of ethyl 5-fluoro-1H-indole-3-carboxylate (200 mg, 0.965 mmol) in 3 mL THF was added with stirring over 30 min at the ice-water bath. Into the flask, (R)-3-bromo-N-(4-cyano-3-(trifluoromethyl)phenyl)-2-hydroxy-2-methylpropanamide (339 mg, 0.965 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=3/2 to produce the designed compound as yellowish solid (289 mg, Yield 63%). The structure of product was confirmed with 2D NMR (COSY and NOESY).

MS (ESI) m/z 476.31 [M−H]⁻; 500.26 [M+Na]⁺; LCMS (ESI) m/z calcd for C₂₃H₁₉F₄N₃O₄476.1390[M−H]⁻, found 476.1301 [M−H]⁻; ¹H NMR (CDCl₃, 400 MHz) δ 8.90 (bs, 1H, NH), 7.98 (s, 1H), 7.88 (d, J=1.6 Hz, 1H), 7.77-7.73 (m, 2H), 7.65 (dd, J=9.4, 2.6 Hz, 1H), 7.42 (dd, J=9.4, 4.0 Hz, 1H), 6.99 (dt, J=8.8, 2.6 Hz, 1H), 4.66 (d, J=14.8 Hz, 1H), 4.39 (d, J=14.8 Hz, 1H), 4.17 (q, J=7.0 Hz, 2H), 4.00 (s, OH), 1.68 (s, 3H), 1.38 (t, J=7.0 Hz, 3H). ¹⁹F NMR (CDCl₃, 400 MHz) δ −62.26, −120.93.

### (S)-N-(4-Cyano-3-(trifluoromethyl)phenyl)-3-(5-fluoro-3-formyl-1H-indol-1-yl)-2-hydroxy-2-methylpropanamide (C₂₁H₁₅F₄N₃O₃) (49)

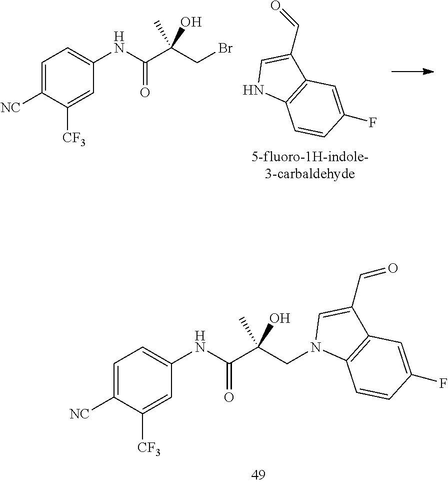

To a dry, nitrogen-purged 100 mL round-bottom flask equipped with a dropping funnel under argon atmosphere, NaH of 60% dispersion in mineral oil (240 mg, 6 mmol) was added in 10 mL of anhydrous THF solvent in the flask at ice-water bath, a solution of 5-fluoro-1H-indole-3-carbaldehyde (326 mg, 2 mmol) in 5 mL THF was added with stirring over 30 min at the ice-water bath. Into the flask, (R)-3-bromo-N-(4-cyano-3-(trifluoromethyl)phenyl)-2-hydroxy-2-methylpropanamide (702 mg, 6 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/ hexane=3/2 to produce the designed compound as yellowish solid (Yield 73%). Purity: 99.14%.

The structure of product was confirmed with 2D NMR (COSY and NOESY). UV max 195.45, 265.45; MS (ESI) m/z 432.21 [M−H]⁻, 456.21 [M+Na]⁺; LCMS (ESI) m/z calcd for C₂₁H₁₅F₄N₃O₃456.0947 [H+Na]⁺, found 456.0887 [M+Na]⁺;¹H NMR (DMSO-d₆, 400 MHz) δ 10.35 (bs, 1H, NH), 9.90 (bs, 1H, CHO), 8.25 (s, 1H), 8.24 (d, J=2.0 Hz, 1H), 8.11 (dd, J=8.4, 2.0 Hz, 1H), 8.05 (d, J=8.4 Hz, 1H), 7.70 (dd, J=9.0, 2.3 Hz, 1H), 7.65 (dd, J=9.0, 4.4 Hz, 1H), 7.65 (dt, J=9.2, 2.8 Hz, 1H), 6.51 (s, OH), 4.69 (d, J=14.4 Hz, 1H), 4.42 (d, J=14.4 Hz, 1H), 1.47 (s, 3H). ¹⁹F NMR (DMSO-d₆, 400 MHz) δ −61.21, -1201.26.

### (S)-N-(4-Cyano-3-(trifluoromethyl)phenyl)-3-(5-fluoro-3-((hydroxyimino)methyl)-1H-indol-1-yl)-2-hydroxy-2-methylpropanamide (C21H16F4N403) (50)

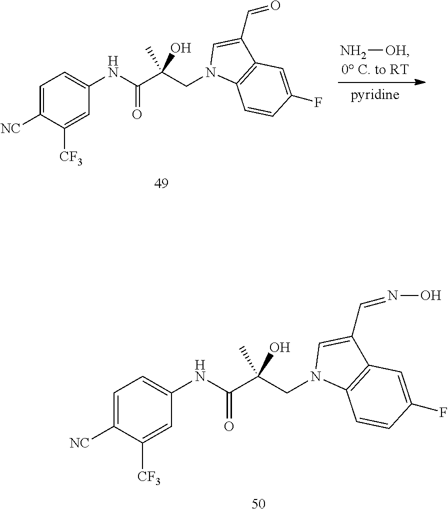

Hydroxylamine hydrochloride (111 mg, 1.5 mmol) was added to a solution of 49 (300 mg, 0.69 mmol) in pyridine (5 mL). The solution was stirred overnight at RT and, then evaporated to dryness. The residue was dissolved in ethyl acetate (20 mL) and H₂O (10 mL) was added to the solution. The residue was filtrated and washed with water to afford pink solid (286 mg, 94% yield). The structure of product was confirmed with 2D NMR (COSY and NOESY). MS (ESI) m/z 447.21 [M−H] −; 449.24 [M+H]⁺; 471.23 [M+Na]⁺; HRMS (ESI) m/z calcd for C₂₁H₁₆F₄N₄O₄449.1237 [M−H]⁻, found 449.1235 [M−H]⁺; ¹H NMR (DMSO-d₆,400 MHz) δ 11.24 (bs, 1H, NH), 10.34 (bs, 1H, OH), 8.34 (s, 1H), 8.24 (s, 1H), 8.10 (d, J=8.6 Hz, 1H), 8.02 (d, J=8.6 Hz, 1H), 7.73 (s, 1H), 7.65 (dd, J=10.0, 2.4 Hz, 1H), 7.55 (dd, J=9.2, 4.4 Hz, 1H), 6.97 (dt, J=9.2, 2.4 Hz, 1H), 6.46 (s, OH), 4.60 (d, J=14.8 Hz, 1H), 4.35 (d, J=14.8 Hz, 1H), 1.43 (s, 3H). ¹⁹F NMR (DMSO-d₆, 400 MHz) δ −61.17, −123 .69.

### (S)-3-(3((2-Acetylhydrazono)methyl)-5-fluoro-1H-indol-1-yl)-N-(4-cyano-3-(trifluoromethyl)phenyl)-2-hydroxy-2-methylpropanamide (C₂₃H₁₉F₄N₅O₃) (51)

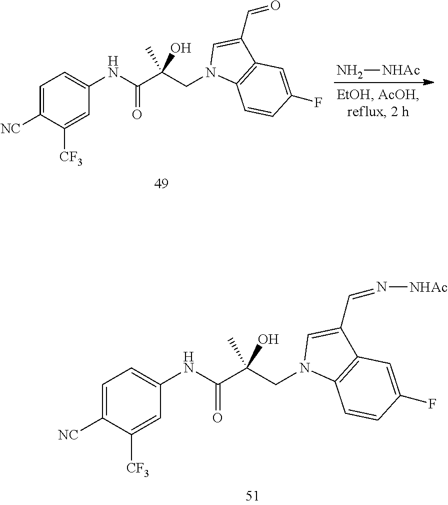 [SMILES: C[C@](O)(Cn1cc(C=O)c2cc(F)ccc21)C(=O)Nc1ccc(C#N)c(C(F)(F)F)c1 ; CC(=O)NN ; CC(=O)N/N=C\c1cn(C[C@](C)(O)C(=O)Nc2ccc(C#N)c(C(F)(F)F)c2)c2ccc(F)cc12]

Acetohydrazine (31 mg, 0.381 mmol) was added to a solution of 49 (150 mg, 0.346 mmol) in ethyl alcohol (10 mL) and a catalytic amount of acetic acid. The solution was heated to reflux for 2 h until starting aldehyde disappeared on TLC. The solution was condensed under reduced pressure, dispersed into ethyl acetate (20 mL), rinsed with saturated sodium chloride. And the solution was dried over anhydrous magnesium sulfate and concentrated under reduced pressure to give crude solid. The mixture was then purified by silica gel column chromatography eluting with ethyl acetate/hexane=3:2, v/v, to give desired product as yellowish solid (153 mg, 90.5% yield).

The structure of product was confirmed with 2D NMR (COSY and NOESY). MS (ESI) m/z 488.26 [M−H]⁻; 490.28 [M+H]⁺, 512.23 [M+Na]⁺; HRMS (ESI) m/z calcd for C₂₃H₁₉F₄N₅O₃ 490.1502 [M+H]⁺, 512.1322 [M+Na]⁺, found 490.1509 [M+H]⁺, 512.1328 [M+Na]⁺; ¹H NMR (CDCl₃, 400 MHz) δ 8.62 (s, 1H), 8.17 (bs, 1H, NHC(O)—), 7.83 (m, 1H, ArH), 7.72 (s, 2H, ArH), 7.54 (m, 2H, ArH), 7.52 (s, 1H, ArH), 7.42 (dd, J=9.2, 4.2 Hz, 1H, ArH), 7.01 (m, 1H, —C═N—NH—), 4.64 (s, 10H), 4.62 (d, J=14.8 Hz, 1H), 4.37 (d, J=14.8 Hz, 1H), 2.30 (s, 3H), 1.66 (s, 3H); ¹⁹F NMR (CDCl₃, 400 MHz) δ -62.22, -121.58. HPLC: tR 3.22 min, purity=99.33%.

### (S)-N-(6-Cyano-5-(trifluoromethyl)pyridin-3-yl)-3-(5-fluoro-3-formyl-1H-indol-1-yl)-2-hydroxy-2-methylpropanamide (C₂₀H₁₄F₄N₄O₃) (53)

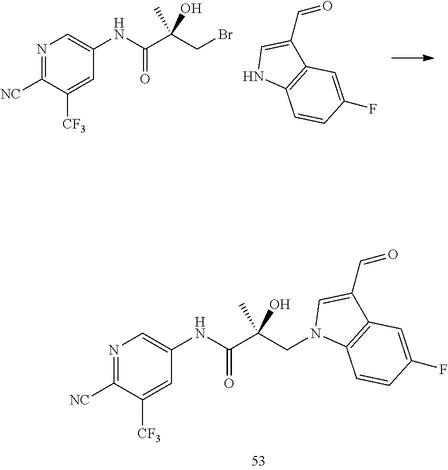

To a dry, nitrogen-purged 100 mL round-bottom flask equipped with a dropping funnel under argon atmosphere, NaH of 60% dispersion in mineral oil (180 mg, 4.5 mmol) was added in 10 mL of anhydrous THF solvent in the flask at ice-water bath, and a solution of 5-fluoro-1H-indole-3-carbaldehyde (245 mg, 1.5 mmol) in 5 mL THF was added with stirring over 30 min at the ice-water bath. Into the flask, (R)-3-bromo-N-(6-cyano (trifluoromethyl)pyridin-3-yl)-2-hydroxy-2-methylpropanamide (528 mg, 1.5 mmol) in 10 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/ hexane=3/2 to produce the designed compound as yellowish solid (Yield 68%).

Purity: 99.14%; the structure of product was confirmed with 2D NMR (COSY and NOESY). MS (ESI) m/z 457.16 [M+Na]⁺, 433.17 [M−H]⁺; LCMS (ESI) m/z calcd for C₂₀H₁₄F₄N₄O₃435.3517 [M+H]⁺, found 435.3538 [M+H]⁺; ¹H NMR (Acetone-d₆, 400 MHz) δ 10.08 (bs, 1H, NH), 9.95 (bs, 1H, CHO), 9.14 (s, 1H), 8.69 (s, 1H), 7.80 (dd, J=9.4, 2.4 Hz, 1H), 7.66 (dd, J=9.2, 4.4 Hz, 1H), 7.05 (dt, J=9.2, 2.4 Hz, 1H), 5.85 (s, OH), 4.86 (d, J=14.8 Hz, 1H), 4.55 (d, J=14.8 Hz, 1H), 1.68 (s, 3H). ¹⁹F NMR (Acetone-d₆, 400 MHz) δ 114.53, 54.70.

### (S)-N-(4-Cyano-3-(trifluoromethyl)phenyl)-3-(3-cyano-5-fluoro-1H-indol-1-yl)-2-hydroxy-2-methylpropanamide (C₂₁H₁₄F₄N₄O₂) (54)

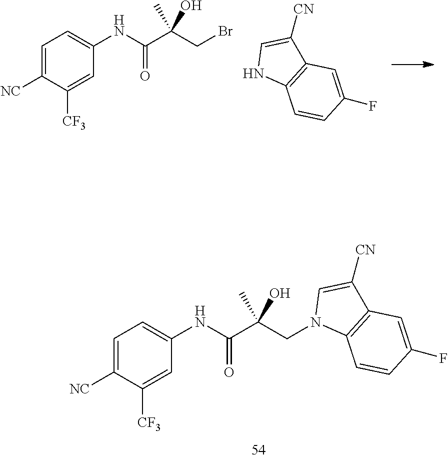

To a dry, nitrogen-purged 100 mL round-bottom flask equipped with a dropping funnel under argon atmosphere, NaH of 60% dispersion in mineral oil (120 mg, 3 mmol) was added in 10 mL of anhydrous THF solvent in the flask at ice-water bath, and a solution of 5-fluoro-1H-indole-3-carbonitrile (160 mg, 1 mmol) in 5 mL THF was added with stirring over 30 min at the ice-water bath. Into the flask, (R)-3-bromo-N-(4-cyano-3-(trifluoromethyl)phenyl)-2-hydroxy-2-methylpropanamide(351 mg, 1 mmol) in 5 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystalized with EtOAc/hexane to produce the designed compound as brownish solid (Yield 63%).

Purity: 98.41%; the structure of product was confirmed with 2D NMR (COSY and NOESY). UV max 196.45, 270.45; MS (ESI) m/z 429.24 [M−H]−, 453.20 [M+Na]⁺; LCMS (ESI) m/z calcd for C₂₁H₁₄F₄N₄O₂431.1131 [M+H]⁺, found 431.1112 [M+H]⁺, 453.1145 [M+Na]⁺; ¹H NMR (DMSO-d₆, 400 MHz) δ 10.32 (bs, 1H, NH), 8.20 (d, J=1.6 Hz, 1H), 8,19 (s, 1H), 8.07 (dd, J=8.6, 1.6 Hz, 1H), 8.02 (d, J=8.6 Hz, 1H), 7.67 (dd, J=9.2, 4.4 Hz, 1H), 7.33 (dd, J=9.2, 2.4 Hz, 1H), 7.08 (dt, J=9.2, 2.4 Hz, 1H), 6.51 (bs, OH), 4.67 (d, J=14.4 Hz, 1H), 4.37 (d, J=14.4 Hz, 1H), 1.43 (s, 3H). ¹⁹F NMR (DMSO-d₆, 400 MHz) δ −61.23, −121.31.

### (S)-N-(6-Cyano-5-(trifluoromethyl)pyridin-3-yl)-3-(5-fluoro-3-((hydroxyimino)methyl)-1H-indol-1-yl)-2-hydroxy-2-methylpropanamide (C₂₀H₁₅F₄N₅O₃) (55)

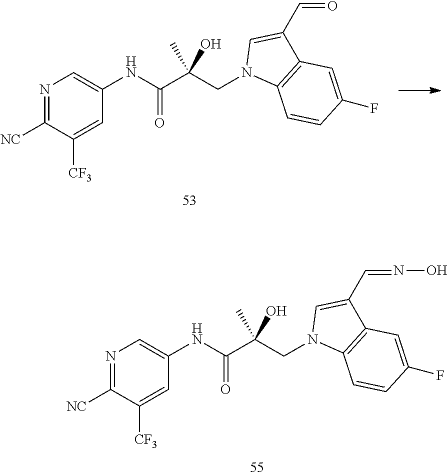 [SMILES: C[C@](O)(Cn1cc(C=O)c2cc(F)ccc21)C(=O)Nc1cnc(C#N)c(C(F)(F)F)c1 ; C[C@](O)(Cn1cc(C=NO)c2cc(F)ccc21)C(=O)Nc1cnc(C#N)c(C(F)(F)F)c1]

Hydroxylamine hydrochloride (111 mg, 1.5 mmol) was added to a solution of 53 (300 mg, 0.69 mmol) in pyridine (5 mL). The solution was stirred overnight at RT and, then evaporated to dryness. The residue was dissolved in ethyl acetate (20 mL) and H₂O (10 mL) was added to the solution. The residue was filtered and washed with water to afford pink solid (286 mg, 94% yield).

The structure of product was confirmed with 2D NMR (COSY and NOESY). UV max 195.45, 266.45.MS (ESI) m/z 448.19 [M−H]−, 450.22 [M+H]⁺; LCMS (ESI) m/z calcd for C₂₀H₁₅F₄N₅O₃ 450.1189 [M+H]⁺, found 450.1192 [M+H]⁺; ¹H NMR (CDCl₃, 400 MHz) δ 9.73 (bs, 1H, NH), 9.15 (bs, 1H, OH), 8.87 (d, J=2.8 Hz, 1H), 8.80 (d, J=2.0 Hz, 1H), 8.64 (d, J=2.0 Hz, 1H), 7.71 (s, 1H), 7.34-7.28 (m, 2H), 7.02 (dt, J=8.6 Hz, 1H), 8.02 (d, J=8.6 Hz, 1H), 7.73 (s, 1H), 7.65 (dd, J=10.0, 2.4 Hz, 1H), 7.55 (dd, J=9.2, 4.4 Hz, 1H), 6.97 (dt, J=8.8, 2.4 Hz, 1H), 6.46 (s, OH), 4.53 (d, J=13.2 Hz, 1H), 4.35 (d, J=13.2 Hz, 1H), 1.57 (s, 3H). ¹⁹F NMR (CDCl₃, 400 MHz) δ −62.18, −121.67.

### (S)-N-(6-Cyano-5-(trifluoromethyl)pyridin-3-yl)-3-(3-cyano-5-fluoro-1H-indol-1-yl)-2-hydroxy-2-methylpropanamide (C₂₀H₁₃F₄N₅O₂) (56)

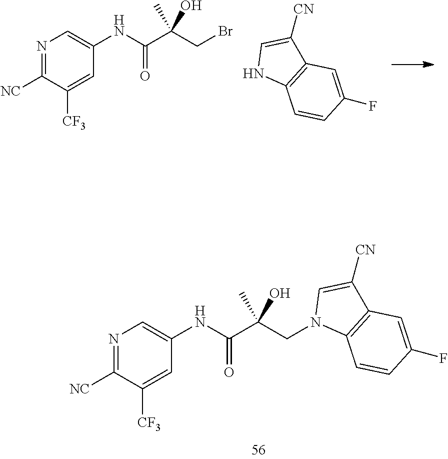

To a dry, nitrogen-purged 100 mL round-bottom flask equipped with a dropping funnel under argon atmosphere, NaH of 60% dispersion in mineral oil (240 mg, 6 mmol) was added in 10 mL of anhydrous THF solvent in the flask at ice-water bath, and a solution of 5-fluoro-1H-indole-3-carbonitrile (320 mg, 2 mmol) in 5 mL THF was added with stirring over 30 min at the ice-water bath. Into the flask, (R)-3-bromo-N-(6-cyano-5-(trifluoromethyl)pyridin-3-yl)-2-hydroxy-2-methylpropanamide (704 mg, 2 mmol) in 5 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with EtOAc/ hexane (1:1, v/v) to produce the designed compound as yellowish solid (Yield 37%).

Purity: 98.78%; MS (ESI) m/z 430.21 [M−H]−, 432.22 [H+M]⁺; LCMS (ESI) m/z calcd for C₂₀H₁₃F₄N₅O₂ 432.1084 [M+H]⁺, found 432.1055 [M+H]⁺; 454.0878 [M+Na]⁺; ¹H NMR (DMSO-d₆, 400 MHz) δ 10.54 (bs, 1H, NH), 9.14 (d, J=2.0 Hz, 1H), 8.52 (d, J=2.0 Hz, 1H), 8.20 (s, 1H), 7.65 (dd, J=9.2, 4.4 Hz, 1H), 7.33 (dd, J=9.2, 2.4 Hz, 1H), 7.08 (dt, J=9.2, 2.4 Hz, 1H), 6.58 (bs, OH), 4.69 (d, J=14.4 Hz, 1H), 4.38 (d, J=14.4 Hz, 1H), 1.45 (s, 3H). ¹⁹F NMR (DMSO-d₆, 400 MHz) δ −61.38, −121.27.

### (S)-N-(4-Cyano-3-(trifluoromethyl)phenyl)-3-(5-fluoro-3-nitro-1H-indol-1-yl)-2-hydroxy-2-methylpropanamide (C₂₀H₁₄F₄N₄O₄) (57)

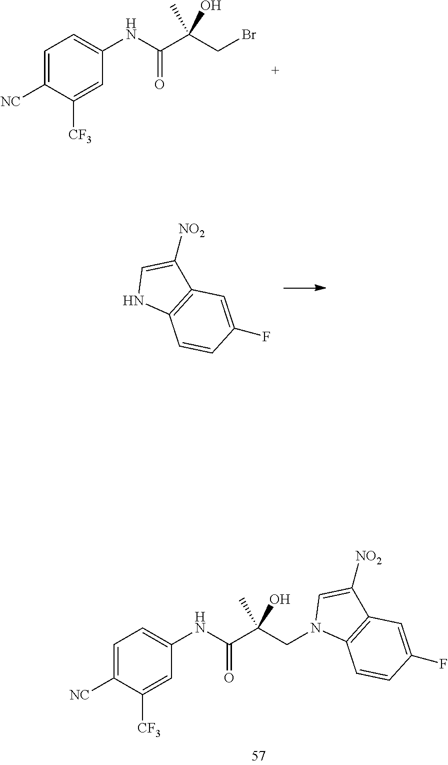 [SMILES: C[C@](O)(CBr)C(=O)Nc1ccc(C#N)c(C(F)(F)F)c1 ; O=[N+]([O-])c1c[nH]c2ccc(F)cc12 ; C[C@](O)(Cn1cc([N+](=O)[O-])c2cc(F)ccc21)C(=O)Nc1ccc(C#N)c(C(F)(F)F)c1]

To a dry, nitrogen-purged 100 mL round-bottom flask equipped with a dropping funnel under argon atmosphere, NaH of 60% dispersion in mineral oil (120 mg, 3 mmol) was added in 10 mL of anhydrous THF solvent in the flask at ice-water bath, and a solution of 5-fluoro-3-nitro-1H-indole (180 mg, 1 mmol) in 5 mL THF was added with stirring over 30 min at the ice-water bath. Into the flask, (R)-3-bromo-N-(4-cyano-3-(trifluoromethyl)phenyl)-2-hydroxy-2-methylpropanamide (351 mg, 1 mmol) in 5 mL of anhydrous THF was added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with EtOAc/ hexane (1:1, v/v) to produce the designed compound as yellowish solid (Yield 42%).

MS (ESI) m/z 449.20 [M−H]⁻, 451.21 [M+H]⁺; LCMS (ESI) m/z calcd for C₂₀H₁₄F₄N₄O₄451.1029 [M+H]⁺, found 451.1026 [M+H]⁺; ¹H NMR (Acetone-d₆, 400 MHz) δ 9.88 (bs, 1H, NH), 8.47 (s, 1H), 8.22 (d, J=2.0 Hz, 1H), 8.10 (dd, J=8.6, 2.0 Hz, 1H), 7.95 (d, J=8.6 Hz, 1H), 7.74 (dd, J=9.2, 3.2 Hz, 1H), 7.11 (dt, J=9.2, 2.4 Hz, 1H), 5.83 (bs, OH), 4.93 (d, J=14.4 Hz, 1H), 4.56 (d, J=14.4 Hz, 1H), 1.67(s, 3H). ¹⁹F NMR (DMSO-d₆, 400 MHz) δ −62.98, −122.77.

### (S)-N-(4-Cyano-3-(trifluoromethyl)phenyl)-3-(5-fluoro-3-(2-(2-(((7-nitrobenzo[c][1,2,5]oxadiazol-4-yl)methyl)amino)acetyl)hydrazono)methyl)-1H-indol-1-yl)-2-hydroxy-2-methylpropanamide (C₃₀H₂₃F₄N₉O₆) (58)

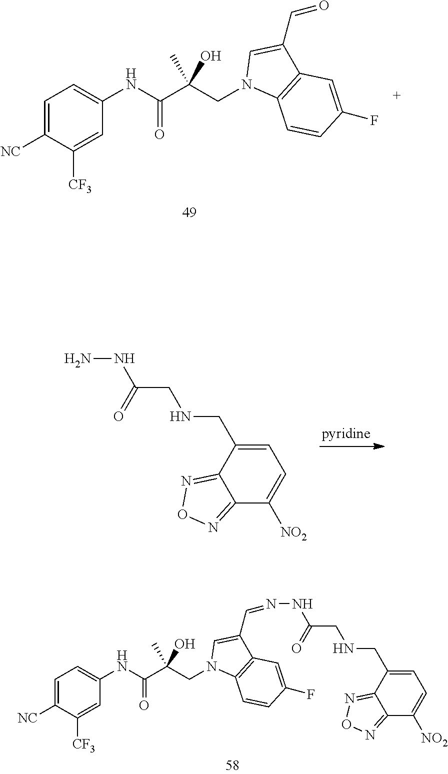 [SMILES: C[C@](O)(Cn1cc(C=O)c2cc(F)ccc21)C(=O)Nc1ccc(C#N)c(C(F)(F)F)c1 ; NNC(=O)CNCc1ccc([N+](=O)[O-])c2nonc12 ; C[C@](O)(Cn1cc(/C=N\NC(=O)CNCc2ccc([N+](=O)[O-])c3nonc23)c2cc(F)ccc21)C(=O)Nc1ccc(C#N)c(C(F)(F)F)c1]

2-[N-(7-Nitro-4-benzofurazanyl)methylamino]acethydrazide (25 mg, 85 mmol) was added to a solution of 49 (37 mg, 85 mmol) in pyridine (5 mL). The solution was stirred overnight at RT and, then evaporated to dryness. The mixture was then purified by silica gel column chromatography eluting with DCM/AcCN=3:1, v/v, to give desired product as orange solid (43 mg, 75% yield). The structure of product was confirmed with 2D NMR (COSY and NOESY).

MS (ESI) m/z 680.33 [M−H]⁻; 681.20 [M+Na]⁺; HRMS (ESI) m/z calcd for C₃₀H₂₃F₄N₉O₆704.1605 [M+Na]⁺, found 704.1631 [M+Na]⁺; ¹H NMR (Acetone-d₆,400 MHz) δ 13.1 (bs, 1H, C(O)NH), 9.86 (bs, 1H, N-NH), 8.54 (d, J=8.8 Hz, 1H), 8.32 (s, 1H), 8.26 (s, 1H), 8.15 (d, J=8.4 Hz, 1H), 7.98 (d, J=8.4 Hz, 1H), 7.84 (dd, J=9.2, 2.4 Hz, 1H), 6.53 (d, J=8.8 Hz, 1H), 5.62 (s, 2H), 5.60 (s, OH), 4.76 (d, J=14.4 Hz, 1H), 4.46 (d, J=14.4 Hz, 1H), 3.60 (bs, NH), 2.82 (s, 2H), 1.63 (s, 3H). ¹⁹F NMR (Acetone-d₆, 400 MHz) δ −62.81, −124.03.

### General Synthesis of aryl (S)-(3-substituted-5-fluoro-1H-indazol-2-hydroxy-2-methylpropanamides

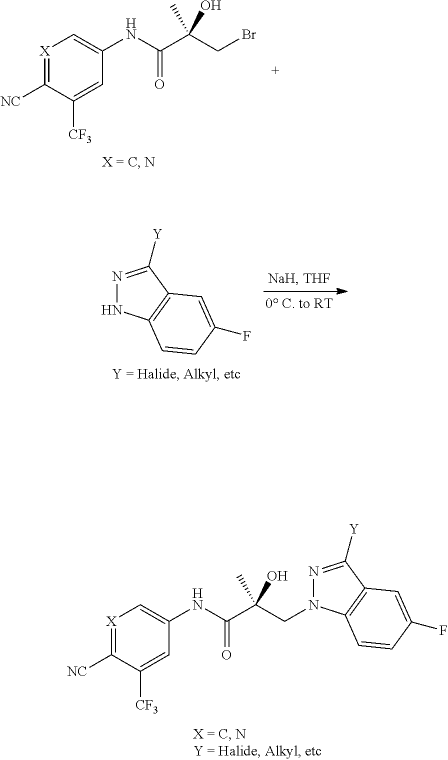

Synthetic Procedure: To a dry, nitrogen-purged 100 mL round-bottom flask equipped a dropping funnel under argon atmosphere, NaH of 60% dispersion in mineral oil (240 mg, 6 mmol) was added in 10 mL of anhydrous THF solvent in the flask at ice-water bath, and a solution of various 3-substituted indazoles (2 mmol) in 5 mL THF were added and stirred 30 min at the ice-water bath. Into the flask, various aryl (R)-3-bromo-2-hydroxy-2-methylpropanamides (2 mmol) in 5 mL of anhydrous THF were added through dropping funnel under argon atmosphere at the ice-water bath and stirred overnight at RT. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with EtOAc/hexane to produce the designed compound. If necessary, an additional recrystallization with EtOAc/hexane was performed to get more the desired product. The structures of all products were confirmed with 2D NMR (COSY and NOESY).

### (S)-3-(3-Chloro-5-fluoro-1H-indazol-1-yl)-N-(4-cyano-3-(trifluoromethyl)phenyl)-2-hydroxy-2-methylpropanamide (99A)

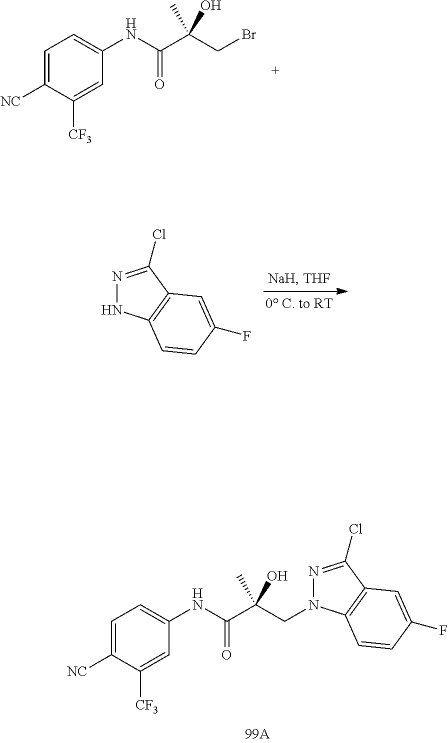 [SMILES: C[C@](O)(CBr)C(=O)Nc1ccc(C#N)c(C(F)(F)F)c1 ; Fc1ccc2[nH]nc(Cl)c2c1 ; C[C@](O)(Cn1nc(Cl)c2cc(F)ccc21)C(=O)Nc1ccc(C#N)c(C(F)(F)F)c1]

White solid; Yield: 58%; Purity: 95.03%; MS (ESI) m/z 439.20 [M−H]−, LCMS (ESI) m/z calcd for C₁₉H₁₃ClF₄N₄O₂ 441.0741 [M+H]⁺, found 441.0703 [M+H]⁺; ¹H NMR (Acetone-d₆, 400 MHz) δ 9.83 (bs, 1H, NH), 8.36 (d, J=2.0 Hz, 1H), 8,18 (dd, J=8.8, 2.0 Hz, 1H), 8.00 (d, J=8.8 Hz, 1H), 7.77 (dd, J=10.0, 4.0 Hz, 1H), 7.32-7.28 (m, 2H), 5.44 (bs, OH), 4.90 (d, J=14.8 Hz, 1H), 4.62 (d, J=14.8 Hz, 1H), 1.61 (s, 3H); ¹⁹F NMR (Acetone-d₆, 400 MHz) δ −62.82, −122.89.

### (S)-3-(3-Chloro-5-fluoro-1H-indazol-1-yl)-N-(6-cyano-5-(trifluoromethyl)pyridin-3-yl)-2- hydroxy-2-methylpropanamide (99B)

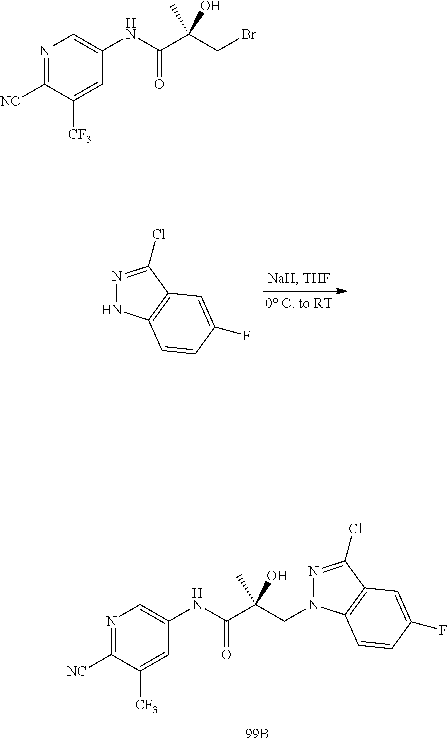 [SMILES: C[C@](O)(CBr)C(=O)Nc1cnc(C#N)c(C(F)(F)F)c1 ; Fc1ccc2[nH]nc(Cl)c2c1 ; C[C@](O)(Cn1nc(Cl)c2cc(F)ccc21)C(=O)Nc1cnc(C#N)c(C(F)(F)F)c1]

White solid;Yield: 88%; Purity: 97.54%; MS (ESI) m/z 440.09 [M−H]−, 442.41 [M+H]⁺; LCMS (ESI) m/z calcd for C₁₈H₁₂ClF₄N₅O₂442.0694 [M+H]⁺, 464.0513 [M+Na]⁺, found 442.0652 [M+H]⁺, 464.0510 [M+Na]⁺; ¹H NMR (Acetone-d₆, 400 MHz) δ 10.03 (bs, 1H, NH), 9.19 (d, J=1.8 Hz, 1H), 8,80 (d, J=1.8 Hz, 1H), 7.76 (m, 1H), 7.73-2.30 (m, 2H), 5.53 (bs, OH), 4.89 (d, J=14.8 Hz, 1H), 4.65 (d, J=14.8 Hz, 1H), 1.63 (s, 3H); ¹⁹F NMR (Acetone-d₆, 400 MHz) δ −62.93, −122.86.

### (S)-N-(6-Cyano-5-(trifluoromethyl)pyridin-3-yl)-3-(5-fluoro-3-iodo-1H-indazol-1-yl)-2-hydroxy-2-methylpropanamide (99C)

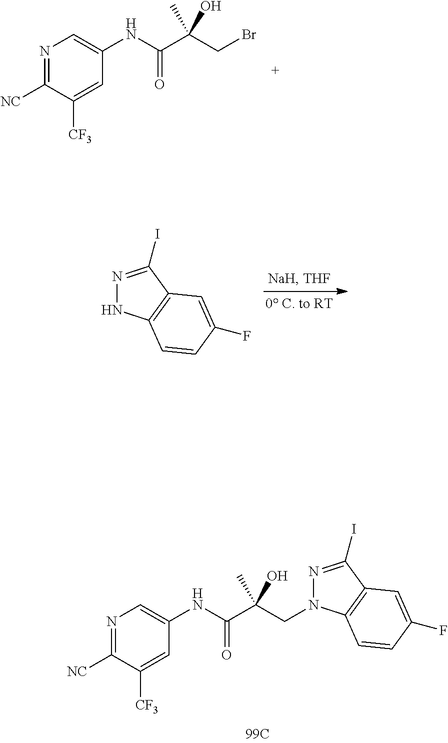 [SMILES: C[C@](O)(CBr)C(=O)Nc1cnc(C#N)c(C(F)(F)F)c1 ; Fc1ccc2[nH]nc(I)c2c1 ; C[C@](O)(Cn1nc(I)c2cc(F)ccc21)C(=O)Nc1cnc(C#N)c(C(F)(F)F)c1]

White solid; Yield: 63%; Purity: 98.95%; MS (ESI) m/z 532.03 [M−H]−, 556.04 [M+Na]⁻; LCMS (ESI) m/z calcd for C₁₈H₁₂F₄IN₅O₂ 534.0050 [M+H]⁺, found 534.0048 [M+H]⁺; ¹H NMR (CDCl₃, 400 MHz) δ 9.23 (bs, 1H, NH), 8.78 (d, J=2.4 Hz, 1H), 8,54 (d, J=2.4 Hz, 1H), 7.47 (dd, J=9.2, 3.6 Hz, 1H), 7.30 (dt, J=8.8, 2.4 Hz, 1H), 7.11 (dd, J=8.0, 2.4 Hz, 1H), 5.56 (bs, OH), 4.95 (d, J=14.0 Hz, 1H), 4.45 (d, J=14.0 Hz, 1H), 1.56 (s, 3H); ¹⁹F NMR (CDCl₃, 400 MHz) δ −62.17, −119.49.

### (S)-3-(3-Bromo-5-fluoro-1H-indazol-1-yl)-N-(6-cyano-5-(trifluoromethyl)pyridin-3-yl)-2-hydroxy-2-methylpropanamide (99D)

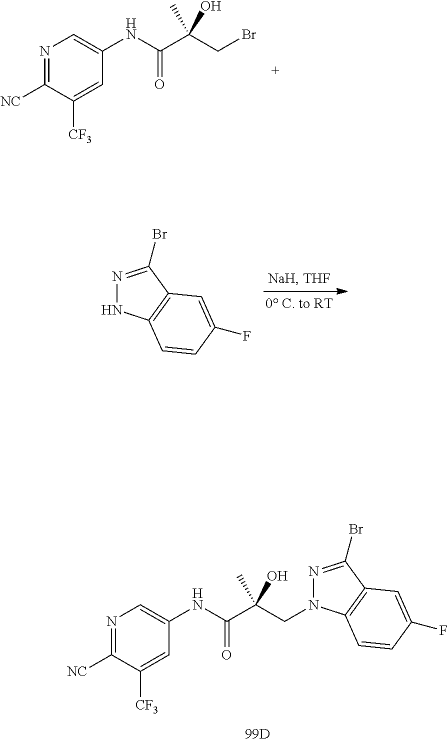 [SMILES: C[C@](O)(CBr)C(=O)Nc1cnc(C#N)c(C(F)(F)F)c1 ; Fc1ccc2[nH]nc(Br)c2c1 ; C[C@](O)(Cn1nc(Br)c2cc(F)ccc21)C(=O)Nc1cnc(C#N)c(C(F)(F)F)c1]

White solid;Yield: 58%; MS (ESI) m/z 485.07 [M−H]−, 487.11 [M+H]⁺; 508.12 [M+Na]⁺; LCMS (ESI) m/z calcd for C₁₈H₁₂BrF₄N₅O₂ 486.0189 [M+H]⁺ found 486.0187 [M+H]⁺; ¹H NMR (CDCl₃, 400 MHz) δ 9.24 (bs, 1H, NH), 8.79 (d, J=2.0 Hz, 1H), 8,54 (d, J=2.0 Hz, 1H), 7.49 (dd, J=8.0, 2.0 Hz, 1H), 7.31-7.27 (m, 1H), 7.26-7.21 (m, 1H), 5.63 (bs, OH), 4.93 (d, J=14.4 Hz, 1H), 4.42 (d, J=14.4 Hz, 1H), 1.58 (s, 3H); ¹⁹F NMR (CDCl₃, 400 MHz) δ −62.17, −119.40.

### (S)-N-(6-Cyano-5-(trifluoromethyl)pyridin-3-yl)-3-(5-fluoro-3-methyl-1H-indazol-1-yl)-2-hydroxy-2-methylpropanamide (99E)

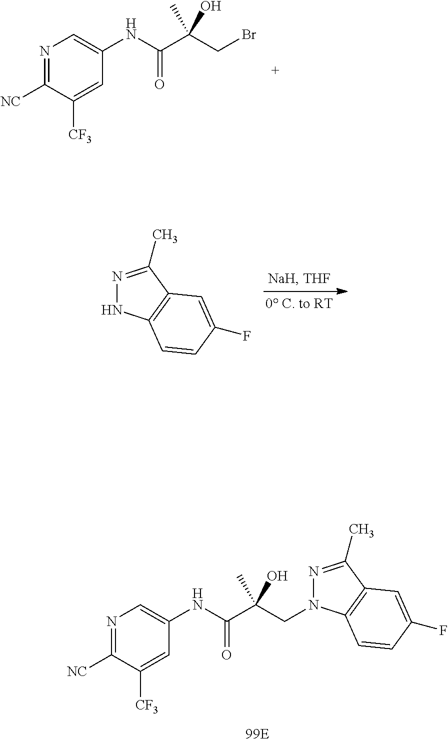 [SMILES: C[C@](O)(CBr)C(=O)Nc1cnc(C#N)c(C(F)(F)F)c1 ; Cc1n[nH]c2ccc(F)cc12 ; Cc1nn(C[C@](C)(O)C(=O)Nc2cnc(C#N)c(C(F)(F)F)c2)c2ccc(F)cc12]

White solid; Yield: 68%; Purity: 98.85% ; MS (ESI) m/z 420.20 [M−H]−, 422.32 [M+H]⁺; 444.26 [M+Na]⁺; LCMS (ESI) m/z calcd for C₁₉H₁₅F₄N₅O₂ 420.1084 [M−H]−; found: 420.1101 [M−H]−; ¹H NMR (CDCl₃, 400 MHz) δ 9.30 (bs, 1H, NH), 8.75 (d, J=2.2 Hz, 1H), 8,56 (d, J=2.2 Hz, 1H), 7.39 (dd, J=8.8, 4.0 Hz, 1H), 7.25-7.19 (m, 2H), 6.38 (bs, OH), 4.85 (d, J=14.0 Hz, 1H), 4.33 (d, J=14.0 Hz, 1H), 2.53 (s, 3H), 1.53 (s, 3H);¹⁹F NMR (CDCl₃, 400 MHz) δ −62.18, −122.23.

### (S)-N-(4-cyano-3-(trifluoromethyl)phenyl)-3-(2,3-dioxoindolin-1-yl)-2-hydroxy-2-methylpropanamide (120)

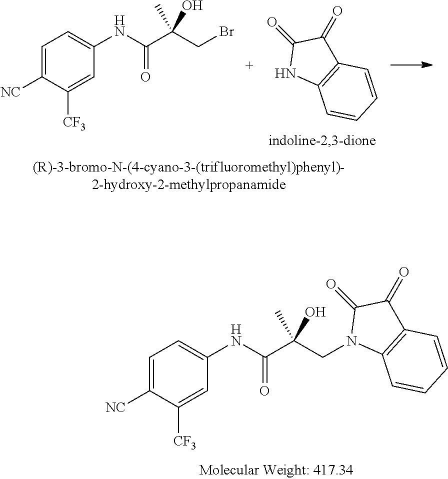 [SMILES: C[C@](O)(CBr)C(=O)Nc1ccc(C#N)c(C(F)(F)F)c1 ; O=C1Nc2ccccc2C1=O ; C[C@](O)(CN1C(=O)C(=O)c2ccccc21)C(=O)Nc1ccc(C#N)c(C(F)(F)F)c1]

To a dry, nitrogen-purged 100 mL round-bottom flask under argon atmosphere, a mixture of indoline-2,3-dione (147 mg, 10.0 mmol,), (R)-3-bromo-N-(4-cyano-3-(trifluoromethyl)phenyl)-2-hydroxy-2-methylpropanamide (351 mg, 10.0 mmol) and K₂CO₃ (14.5 mmol, 277 mg) was stirred in DMF (5 mL) vigorously for 12 hours at room temperature. Poured the reaction mixture into water (50 mL) and extracted with EtOAc (10 mL×3), washed with brine, aqueous ammonium chloride, and water. The ethyl acetate extract was dried by MgSO₄, concentrated under reduced pressure, and purified by flash column chromatography using EtOAc/hexane (1:1, v/v) as an eluent to give the target compound.

Yield: 34%;

Brown gel;

UV max 197.45, 270.45;

HPLC: tR 3.18 min, purity 97.82%;

MS (ESI) m/z 416.2 [M−H]−;

¹H NMR (CDC13, 400 MHz) δ 9.16 (bs, 1H, NH), 8.09 (d, J=1.6 Hz, 1H), 7.92 (d, J=8.4, 1.6 Hz, 1H), 7.79 (d, J=8.4 Hz, 1H), 6.91 (t, J=8.4 Hz, 2H), 6.60 (d, J=8.4 Hz, 1H), 6.56 (d, J=8.4 Hz, 1H), 4.52 (bs, OH), 3.84 (d, J=13.6 Hz, 1H), 3.25 (d, J=13.6 Hz, 1H), 1.57 (s, 3H); ¹³C NMR (CDCl₃,100 MHz) δ 173.9, 158.5, 156.2, 142.7, 141.4, 135.8, 133.9 (q, J=33 Hz), 123.4, 121.8, 120.7, 117.3 (q, J=5 Hz), 116.4, 116.3, 116.1, 115.9, 115.5, 104.6, 60.5, 21.1; ¹⁹F NMR (CDCl₃, 400 MHz) δ −62.21.

### (S)-3-(3-Chloro-4-(trifluoromethyl)-1H-indazol-1-yl)-N-(4-cyano-3-(trifluoromethyl)phenyl)-2-hydroxy-2-methylpropanamide (99G)

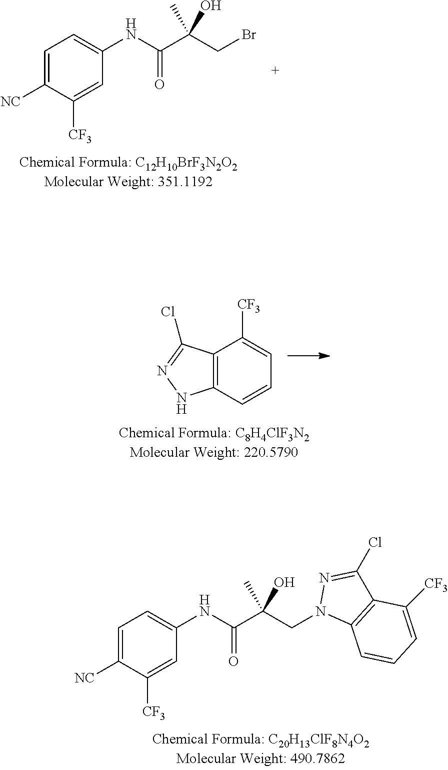 [SMILES: C[C@](O)(CBr)C(=O)Nc1ccc(C#N)c(C(F)(F)F)c1 ; FC(F)(F)c1cccc2[nH]nc(Cl)c12 ; C[C@](O)(Cn1nc(Cl)c2c(C(F)(F)F)cccc21)C(=O)Nc1ccc(C#N)c(C(F)(F)F)c1]

To a dry, nitrogen-purged 100 mL round-bottom flask equipped a dropping funnel under argon atmosphere, NaH of 60% dispersion in mineral oil (240 mg, 3 mmol) was added in 10 mL of anhydrous THF solvent in the flask at ice-water bath, and a solution of 3-Cl 4CF3-indazole (441 mg, 2 mmol) in 5 ml THF was added stirred 30 min at the ice-water bath. Into the flask, (R)-3-bromo-N-(4-cyano-3-(trifluoromethyl)phenyl)-2-hydroxy-2-methylpropanamide (702 mg, 2 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with EtOAc/hexane to produce the designed compound as light yellowish solid. Additional recrystallization with DCM produced white solid. Yield: 68%;

Purity: 98.79%;

UV max 214.45, 271.45.

MS (ESI) m/z 489.20 [M−H]⁻;

LCMS (ESI) m/z calcd for C₂₀H₁₃ClF₆N₄O₂489.0553 [M−H]⁻; found: 489.0582 [M−H]⁻; 491.0709 [M+H]⁺ found 491.0713.

¹H NMR (CDCl₃, 400 MHz) δ 9.08 (bs, 1H, NH), 7.95 (s, 1H), 7.78-7.73 (m, 3H), 7.59-7.55 (m, 2H), 5.31 (bs, OH), 5.00 (d, J=14.2 Hz, 1H), 4.53 (d, J=14.2 Hz, 1H), 1.57 (s, 3H). ¹⁹F NMR (CDCl₃, 400 MHz) δ −58.26, −62.27.

### (S)-N-(3-Chloro-4-cyanophenyl)-3-(3-chloro-5-fluoro-1H-indazol-1-yl)-2-hydroxy-2-methylpropanamide (99F)

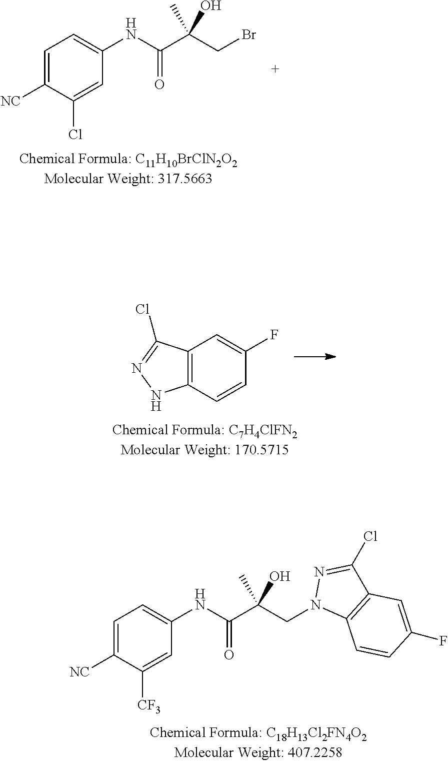 [SMILES: C[C@](O)(CBr)C(=O)Nc1ccc(C#N)c(Cl)c1 ; Fc1ccc2[nH]nc(Cl)c2c1 ; C[C@](O)(Cn1nc(Cl)c2cc(F)ccc21)C(=O)Nc1ccc(C#N)c(C(F)(F)F)c1]

To a dry, nitrogen-purged 100 mL round-bottom flask equipped a dropping funnel under argon atmosphere, NaH of 60% dispersion in mineral oil (240 mg, 3 mmol) was added in 10 mL of anhydrous THF solvent in the flask at ice-water bath, and a solution of 3-C1-5-F-indazole (341 mg, 2 mmol) in 5 ml THF was added stirred 30 min at the ice-water bath. Into the flask, (R)-3-bromo-N-(3 -chloro-4-cyanophenyl)-2-hydroxyl)-2-methylpropanamide DJ-VI-5 (635 mg, 2 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with EtOAc/hexane (2/3, v/v) to produce the designed compound as white solid. Additional recrystallization with DCM produced white solid.

Yield: 67%;

Purity: 97.54%;

UV max 269.45.

MS (ESI) rn/z 405.20 [M−H]⁻;

LCMS (ESI) m/z calcd for C₁₈H₁₃Cl₂FN₄O₂ 405.0321 [M−H]⁻; found: 405.0352 [M−H]⁻;

¹H NMR (CDCl₃, 400 MHz) δ 8.96 (bs, 1H, NH), 7.80 (s, 1H), 7.56 (d, J=8.4 Hz, 1H), 7.48 (m, 1H), 7.38 (d, J=8.4 Hz, 1H), 7.30-7.27 (m, 2H), 5.33 (bs, OH), 4.90 (d, J=14.0 Hz, 1H), 4.36 (d, J=14.0 Hz, 1H), 1.53 (s, 3H). ¹⁹F NMR (CDCl₃, 400 MHz) δ −119.75.

### (S)-3-(3-Chloro-4-(trifluoromethyl)-1H-indazol-1-yl)-N-(6-cyano-5-(trifluoromethyl)pyridin-3-yl)-2-hydroxy-2-methylpropanamide (99H)

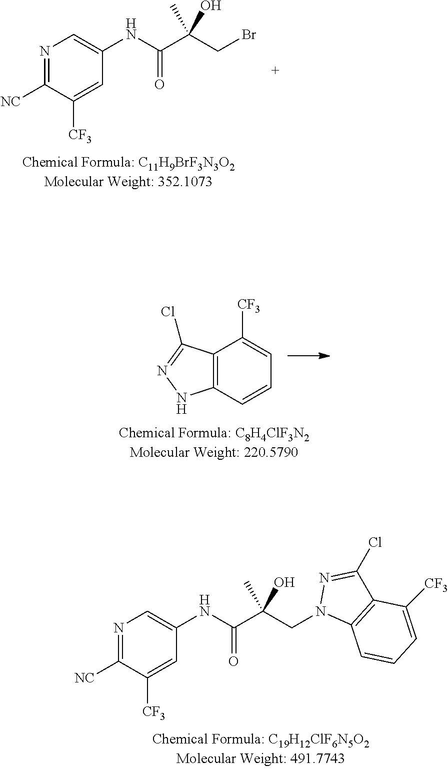 [SMILES: C[C@](O)(CBr)C(=O)Nc1cnc(C#N)c(C(F)(F)F)c1 ; FC(F)(F)c1cccc2[nH]nc(Cl)c12 ; C[C@](O)(Cn1nc(Cl)c2c(C(F)(F)F)cccc21)C(=O)Nc1cnc(C#N)c(C(F)(F)F)c1]

To a dry, nitrogen-purged 100 mL round-bottom flask equipped a dropping funnel under argon atmosphere, NaH of 60% dispersion in mineral oil (240 mg, 6 mmol) was added in 10 mL of anhydrous THF solvent in the flask at ice-water bath, and a solution of 3-Cl 4-CF3-indazole (441 mg, 2 mmol) in 5 ml THF was added stirred 30 min at the ice-water bath. Into the flask, (R)-3-bromo-N-(6-cyano-5-(trifluoromethyl)pyridin-3-yl)-2-hydroxy-2-methylpropanamide (704 mg, 2 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness.

Yield: 48%;

Purity: 99.9%;

UV max: 284.45;

MS (ESI) m/z 490.06 [M−H]⁻;

LCMS (ESI) m/z calcd for C₁₉H₁₂ClF₆N₅O₂490.0505 [M−H]⁻; found:

¹H NMR (CDCl₃, 400 MHz) δ 9.22 (bs, 1H, NH), 8.81 (d, J=2.2 Hz, 1H), 8.54 (d, J=2.2 Hz, 1H), 7.75 (d, J=8.0 Hz, 1H),7.58 (m, 2H), 5.42 (bs, OH), 5.01 (d, J=14.4 Hz, 1H), 4.45 (d, J=14.4 Hz, 1H), 1.59 (s, 3H); ¹⁹F NMR (CDCl₃, 400 MHz) δ −58.22, −62.10.

### (S)-N-(4-Cyano-3-(trifluoromethyl)phenyl)-2-hydroxy-2-methyl-3-(4-sulfamoyl-1H-pyrazol-1-yl)propenamide (1091)

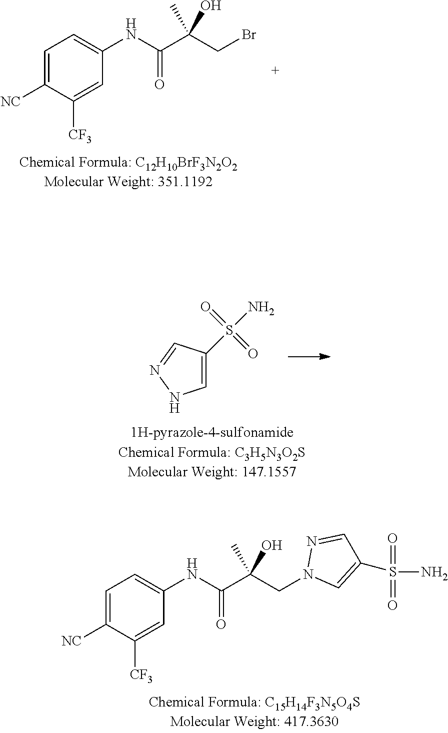 [SMILES: C[C@](O)(CBr)C(=O)Nc1ccc(C#N)c(C(F)(F)F)c1 ; NS(=O)(=O)c1cn[nH]c1 ; C[C@](O)(Cn1cc(S(N)(=O)=O)cn1)C(=O)Nc1ccc(C#N)c(C(F)(F)F)c1]

To a dry, nitrogen-purged 100 mL round-bottom flask equipped a dropping funnel under argon atmosphere, NaH of 60% dispersion in mineral oil (240 mg, 6 mmol) was added in 10 mL of anhydrous THF solvent in the flask at ice-water bath, and a solution of 1H-pyrazole-4-sulfonamide (295 mg, 2 mmol) in 5 ml THF was added stirred 30 min at the ice-water bath. Into the flask, (R)-3- bromo-N-(4-cyano-3-(trifluoromethyl)phenyl)-2-hydroxy-2-methylpropanamide (702 mg, 2 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with Acetone/hexane to produce the target compound as white solid.

Yield: 48%;

Purity: 98.18%;

UV max: 270.45;

MS (ESI) m/z 416.20 [M−H]⁻;

LCMS (ESI) m/z calcd for C₁₅H₁₄F₃N₅O₄S 416.0640 [M−H]⁻; found: 416.0679 [M−H]⁻418.0789 [M+H]⁺, 440.0613 [M+Na]⁺;

¹H NMR (Acetone-d₆, 400 MHz) δ 9.76 (bs, 1H, NH), 8.32 (d, J=1.6 Hz, 1H), 8.11 (dd, J=8.4, 1.6 Hz, 1H), 7.94 (s, 1H), 7.88 (d, J=8.4 Hz, 1H), 6.39 (bs, 2H, SO2NH2), 5.53 (bs, OH), 4.54 (d, J=14.4 Hz, 1H), 4.30 (d, J=14.4 Hz, 1H), 1.38 (s, 3H). ¹⁹F NMR (CDCl₃, 400 MHz) δ −62.80.

### Preparation of Compound 1084

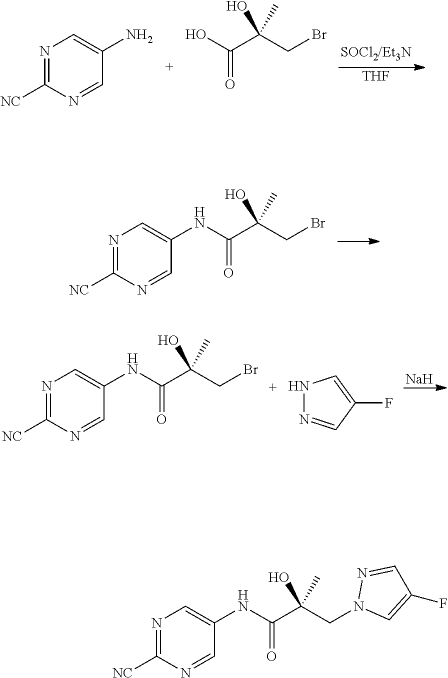

### (R)-3-Bromo-N-(2-Cyanopyrimidin-5-Yl)-2-Hydroxy-2-Methylpropanamide (C₉H₉BrN₄O₂)

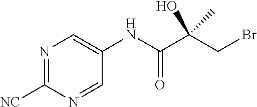 [SMILES: C[C@](O)(CBr)C(=O)Nc1cnc(C#N)nc1]

(R)-3-bromo-2-hydroxy-2-methylpropanoic acid (3.00 g, 0.0163934 mol) reacted with thionyl chloride (2.34 g, 0.01967211 mol), trimethylamine (2.16 g, 0.0213115 mol), and 5-aminopyrimidine-2-carbonitrile (1.97 g, 0.0163934 mol) to afford the title compound. The product was purified by a silica gel column using hexanes and ethyl acetate (1:1) as eluent to afford 3.44 g (73.3%) of the titled compound as yellowish solid.

¹H NMR (400 MHz, DMSO-d₆) δ 10.71 (s, 1H, NH), 9.40-9.37 (m, 2H, ArH), 6.51 (s, 1H, OH), 3.84 (d, J=10.4 Hz, 1H, CH), 3.59 (d, J=10.4 Hz, 1H, CH), 1.50 (s, 3H, CH₃).

Mass (ESI, Positive): [M+H]⁺.

HRMS [C₉H₁₀BrN₁O₂⁺]: calcd 284.9987, found 284.9985 [M+H]⁺. Purity: 97.09% (HPLC).

### (S)-N-(2-Cyanopyrimidin-5-Yl)-2-Methyloxirane-2-Carboxamide (C₉H₈N₄O₂)

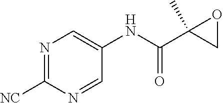

To a solution of (R)-3-bromo-N-(2-cyanopyrimidin-5-yl)-2-hydroxy-2-methylpropanamide (5.00 g, 0.01754 mol) in 25 mL of 2-butanone, was added potassium carbonate (6.06 g, 0.04384 mol). The resulting reaction mixture was heated at reflux for 2 h under argon atmosphere. After the end of the reaction was established by TLC, the reaction was cooled to room temperature (rt), filtered through a pad of Celite, and rinsed the pad of Celite with 15 mL of 2-butanone. The filtrate was concentrated under vacuum and dried under 25-30 inches vacuum to provide (S)-N-(2-cyanopyrimidin-5-yl)-2-methyloxirane-2-carboxamide.

¹H NMR (400 MHz, DMSO-d₆) δ 10.38 (s, 1H, NH), 9.27 (br. s, 2H, ArH), 3.11 (d, J=5.2 Hz, 1H, CH), 3.07 (d, J=8.8 Hz, 1H, CH), 1.56 (s, 3H, CH₃).

Mass (ESI, Positive): [M+H]⁺.

HRMS [C₉H₉N₄O₂⁺]: calcd 205.0726, found 205.0721 [M+H]⁺. Purity: 98.93% (HPLC).

### (S)-N-(2-cyanopyrimidin-5-yl)-3-(4-fluoro-1H-pyrazol-1-yl)-2-hydroxy-2-methylpropanamide (C₁₂H₁₁/fn₆O₂) (1084)

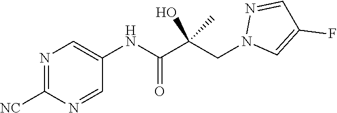 [SMILES: C[C@](O)(Cn1cc(F)cn1)C(=O)Nc1cnc(C#N)nc1]

To a solution of 4-fluoro-1H-pyrazole (0.121 g, 0.001403 mol) in anhydrous THF (10 mL), which was cooled in an ice water bath under an argon atmosphere, was added sodium hydride (60% dispersion in oil, 0.20 g, 0.0049101 mol). After addition, the resulting mixture was stirred for three hours. (R)-3-bromo-N-(2-cyanopyrimidin-5-yl)-2-hydroxy-2-methylpropanamide (0.40 g, 0.001403 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using hexanes and ethyl acetate (1:1) as eluent to afford 0.12 g (33.0%) of the titled compound as off-white solid.

¹H NMR (400 MHz, DMSO-d₆) δ 10.53 (s, 1H, NH), 9.30 (br s, 2H, ArH), 7.75 (d, J=4.4 Hz, 1H, pyrazole-H), 7.42 (d, J=4.0 Hz, 1H, pyrazole-H), 6.41 (s, 1H, OH), 4.38 (d, J=14.0 Hz, 1H, CH), 4.19 (d, J=14.0 Hz, 1H, CH), 1.35 (s, 3H, CH₃).

Mass (ESI, Positive): [M+H]⁺.

HRMS [C₁₂H₁₂FN₆O₂⁺]: calcd 291.1006, found 291.1003 [M+H]⁺. Purity: 98.66% (HPLC).

### (S)-3-(4-Cyano-1H-Pyrazol-1-Yl)-N-(2-Cyanopyrimidin-5-Yl)-2-Hydroxy-2-Methylpropanamide (C₁₃H₁₁N₇O₂) (1085)

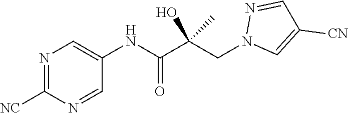 [SMILES: C[C@](O)(Cn1cc(C#N)cn1)C(=O)Nc1cnc(C#N)nc1]

To a solution of 4-cyano-1H-pyrazole (0.131 g, 0.001403 mol) in anhydrous THF (10 mL), which was cooled in an ice water bath under an argon atmosphere, was added sodium hydride (60% dispersion in oil, 0.20 g, 0.0049101 mol). After addition, the resulting mixture was stirred for three hours. (R)-3-bromo-N-(2-cyanopyrimidin-5-yl)-2-hydroxy-2-methylpropanamide (0.40 g, 0.001403 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1) as eluent to afford 0.115 g (27.6%) of the titled compound as yellowish solid.

¹H NMR (400 MHz, DMSO-d₆) δ 10.50 (s, 1H, NH), 9.29 (br s, 2H, ArH), 8.46 (s, 1H, pyrazole-H), 8.00 (s, 1H, pyrazole-H), 6.52 (s, 1H, OH), 4.55 (d, J=14.0 Hz, 1H, CH), 4.37 (d, J=14.0 Hz, 1H, CH), 1.48 (s, 3H, CH₃).

Mass (ESI, Positive): [M+H]⁺.

HRMS [C₁₃H₁₂N₇O₂⁺]: calcd 298.1052, found 298.1055 [M+H]⁺. Purity: 99.26% (HPLC).

### Preparation of Compound 1086

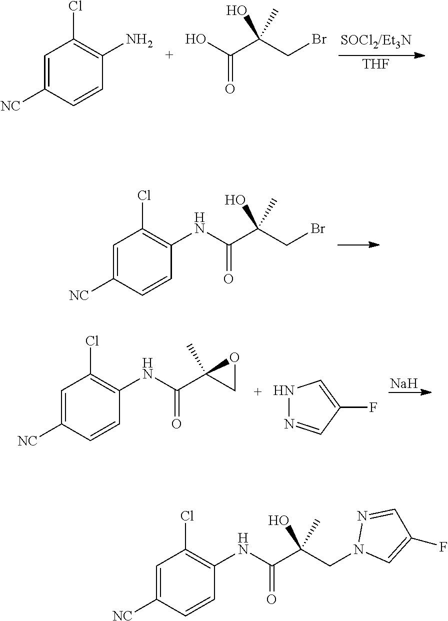

### (R)-3-Bromo-N-(2-Chloro-4-Cyanophenyl)-2-Hydroxy-2-Methylpropanamide (C₁₁H₁₀BrClN₂O₂)

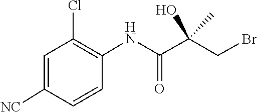

(R)-3-bromo-2-hydroxy-2-methylpropanoic acid (3.33 g, 0.018182 mol) reacted with thionyl chloride (2.60 g, 0.02182 mol), trimethylamine (2.16 g, 0.0213115 mol), and 5- aminopyrimidine-2-carbonitrile (2.39 g, 0.023638 mol) to afford the title compound. The product was purified by a silica gel column using DCM and ethyl acetate (19:1) as eluent to afford 4.02g (69.5%) of the titled compound as yellow solid.

¹H NMR (400 MHz, DMSO-d₆) δ 9.78 (s, 1H, NH), 8.49 (dd, J=8.8 Hz, J=4.4 Hz, 1H, ArH), 8.19 (d, J=1.6 Hz, 1H, ArH), 7.88 (dd, J=8.8 Hz, J=2.0 Hz, 1H, ArH), 6.84 (s, 1H, OH), 3.84 (d, J=10.4 Hz, 1H, CH), 3.59 (d, J=10.4 Hz, 1H, CH), 1.56 (s, 3H, CH3).

Mass (ESI, Positive): [M+H]⁺.

HRMS [C₁₁H₁₁BrClN₂O₂^{+]: calcd} 316.9690, found 316.9684 [M+H]⁺. Purity: 98.38% (HPLC).

### (S)-N-(2-Chloro-4-Cyanophenyl)-2-Methyloxirane-2-Carboxamide (C₁₁H₉ClN₂O₂)

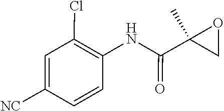 [SMILES: C[C@@]1(C(=O)Nc2ccc(C#N)cc2Cl)CO1]

To a solution of (R)-3-bromo-N-(2-chloro-4-cyanophenyl)-2-hydroxy-2-methylpropanamide (5.00 g, 0.01575 mol) in 25 mL of 2-butanone, was added potassium carbonate (3.26 g, 0.02362 mol). The resulting reaction mixture was heated at reflux for 2 h under argon atmosphere. After the end of the reaction was established by TLC, the reaction was cooled to room temperature (rt), filtered through a pad of Celite, and rinsed the pad of Celite with 15 mL of 2-butanone. The filtrate was concentrated under vacuum, and dried under 25-30 inches vacuum to provide (S)-N-(2-chloro-4-cyanophenyl)-2-methyloxirane-2-carboxamide.

¹H NMR (400 MHz, DMSO-d₆) δ 9.17 (s, 1H, NH), 8.19-8.15 (m, 2H, ArH), 7.87-7.84 (m, 1H, ArH), 3.17 (d, J=5.2 Hz, 1H, CH), 3.08 (d, J=5.2 Hz, 1H, CH), 1.56 (s, 3H, CH₃).

Mass (ESI, Positive): [M+H]⁺.

HRMS [C₁₁H₈ClN₂O₂−]: calcd 235.0274, found 235.0265 [M+H]⁺. Purity: 67.48% (HPLC).

### (S)-N-(2-Chloro-4-Cyanophenyl)-3-(4-Fluoro-1H-Pyrazol-1-Yl)-2-Hydroxy-2-Methylpropanamide (C₁₄H₁₂ClFN₄O₂) (1086)

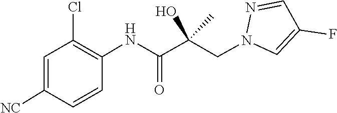

To a solution of 4-fluoro-1H-pyrazole (0.108 g, 0.0012595 mol) in anhydrous THF (10 mL), which was cooled in an ice water bath under an argon atmosphere, was added sodium hydride (60% dispersion in oil, 0.176 g, 0.0044082 mol). After addition, the resulting mixture was stirred for three hours. (R)-3-bromo-N-(2-chloro-4-cyanophenyl) hydroxy-2-methylpropanamide (0.40 g, 0.0012595 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (19:1 to 9:1) as eluent to afford 0.15 g (31.6%) of the titled compound as off-white solid.

¹H NMR (400 MHz, DMSO-d₆) δ 9.50 (br s, 1H, NH), 8.44 (d, J=8.8 Hz, 1H, ArH), 8.15 (d, J=1.6 Hz, 1H, ArH), 7.86 (dd, J=8.8 Hz, J=2.0 Hz, 1H, ArH), 7.75 (d, J=4.8Hz, 1H, pyrazole-H), 7.38 (d, J=4.2 Hz, 1H, pyrazole-H), 6.76 (br s, 1H, OH), 4.39 (d, J=14.0 Hz, 1H, CH), 4.12 (d, J=14.0 Hz, 1H, CH), 1.39 (s, 3H, CH3).

Mass (ESI, Positive): [M+H]⁺.

HRMS [C₁₄H₁₃ClFN₄O₂⁺]: calcd 323.0711, found 323.0723 [M+H]⁺. Purity: 98.81% (HPLC).

### (S)-N-(2-Chloro-4-Cyanophenyl)-3-(4-Cyano-1H-Pyrazol-1-Yl)-2-Hydroxy-2-Methylpropanamide (C₁₅H₁₂ClN₅O₂) (1087)

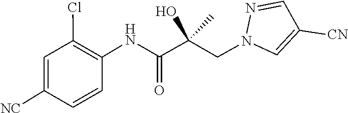

To a solution of 4-cyano-1H-pyrazole (0.117 g, 0.0012595 mol) in anhydrous THF (10mL), which was cooled in an ice water bath under an argon atmosphere, was added sodium hydride (60% dispersion in oil, 0.176 g, 0.0044082 mol). After addition, the resulting mixture was stirred for three hours. (R)-3-bromo-N-(2-chloro-4-cyanophenyl)-2-hydroxy-2-methylpropanamide (0.40 g, 0.0012595 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (19:1 to 9:1) as eluent to afford 0.15 g (31.6%) of the titled compound as off-white solid.

¹H NMR (400 MHz, DMSO-d₆) δ 9.47 (br s, 1H, NH), 8.46 (s, 1H, pyrazole-H), 8.42 (d, J=8.2 Hz, 1H, ArH), 8.15 (d, J=2.0 Hz, 1H, ArH), 7.97 (s, 1H, pyrazole-H), 7.88 (dd, J=8.2 Hz, J=2.0 Hz, 1H, ArH), 6.88 (br s, 1H, OH), 4.56 (d, J=14.0 Hz, 1H, CH), 4.36(d, J=14.0 Hz, 1H, CH), 1.43 (s, 3H, CH3).

Mass (ESI, Positive): [M+H]⁺. HRMS [C₁₅H₁₃ClN₅O₅⁺]: calcd 330.0758, found 330.0753 [M+H]⁺. Purity: 95.75% (HPLC).

### Preparation of Compound 1088

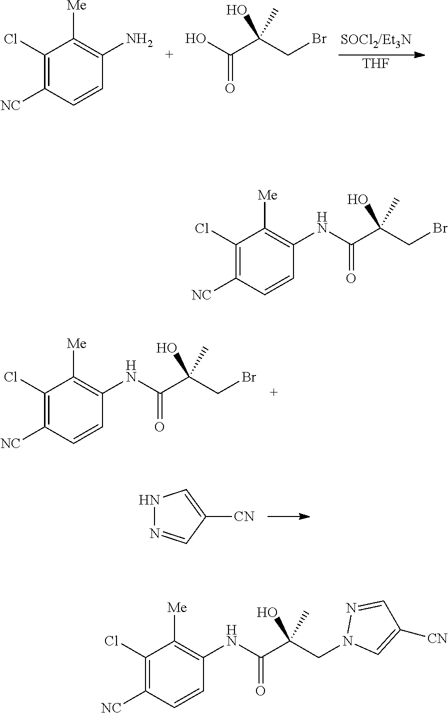

### (R)-3-bromo-N-(3-chloro-4-cyano-2-methylphenyl)-2-hydroxy-2-methylpropanamide (C₁₂H₁₂BrClN₂O₂)

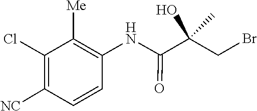 [SMILES: Cc1c(NC(=O)[C@@](C)(O)CBr)ccc(C#N)c1Cl]

(R)-3-bromo-2-hydroxy-2-methylpropanoic acid (1.21 g, 0.006602 mol) reacted with thionyl chloride (0.86 g, 0.007202 mol), trimethylamine (0.79 g, 0.007802 mol), and 4-amino-2-chloro-3-methylbenzonitrile (1.00 g, 0.006002 mol) to afford the title compound. The product was purified by a silica gel column using DCM and ethyl acetate (19:1) as eluent to afford 1.60g (80.4%) of the titled compound as yellow solid.

¹HNMR (400MHz, DMSO-d₆) δ 9.70 (s, 1H, NH), 7.84 (d, J=8.4 Hz, 1H, ArH), 7.76 (d, J=8.4 Hz, 1H, ArH), 6.50(s, 1H, OH), 3.84 (d, J=9.2 Hz, 1H, CH), 3.59(d, J=9.2 Hz, 1H, CH), 2.32 (s, 3H, CH₃), 1.50 (s, 3H, CH₃).

Mass (ESI, Positive): [M+H]⁺.

HRMS [C₁₂H₁₃BrClN₂O₂⁺]: calcd 330.9849, found 330.9843 [M+H]⁺. Purity: 98.80% (HPLC).

### (S)-N-(3-chloro-4-cyano-2-methylphenyl)-3-(4-cyano-1H-pyrazol-1-yl)-2-hydroxy-2-methylpropanamide (C₁₆H₁₄ClN₅O₂) (1088)

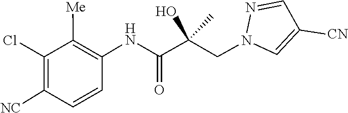

To a solution of 4-cyano-1H-pyrazole (0.112 g, 0.0012063 mol) in anhydrous THF (10 mL), which was cooled in an ice water bath under an argon atmosphere, was added sodium hydride (60% dispersion in oil, 0.169 g, 0.0042217 mol). After addition, the resulting mixture was stirred for three hours. (R)-3-bromo-N-(3-chloro-4-cyano-2-methylphenyl)-2-hydroxy-2-methylpropanamide (0.40 g, 0.0012063 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1 to 5:1) as eluent to afford 0.31 g (74.7%) of the titled compound as off-white solid.

¹H NMR (400 MHz, DMSO-d₆) δ 9.52 (br s, 1H, NH), 8.46 (s, 1H, Pyrazole-H), 8.04(s, 1H, Pyrazole-H), 7.83 (d, J=8.8 Hz, 1H, ArH), 7.78 (d, J=8.8 Hz, 1H, ArH), 6.51 (br s, 1H, OH), 4.56(d, J=14.4 Hz, 1H, CH), 4.34 (d, J=14.0 Hz, 1H, CH), 2.18(s, 3H, CH₃), 1.40 (s, 3H, CH₃).

Mass (ESI, Positive): [M+H]⁺.

HRMS [C₁₆H₁₅ClN₅O₂⁺]: calcd 344.0914, found 344.0910 [M+H]⁺. Purity: 99.59% (HPLC).

### Preparations of Compounds 1089 & 1090

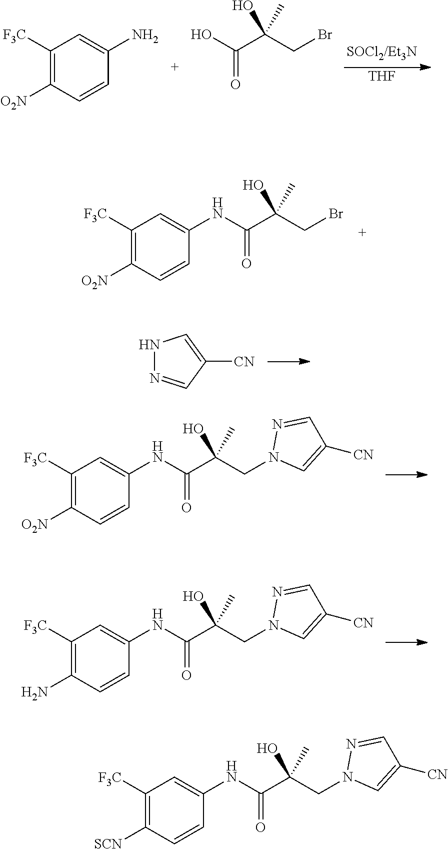

### (R)-3-bromo-2-hydroxy-2-methyl-N-(4-nitro-3-(trifluoromethyl)phenyl)propanamide (C₁₁H₁₀BrF₃N₂O₄)

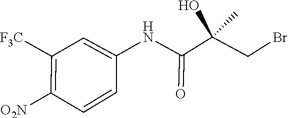 [SMILES: C[C@](O)(CBr)C(=O)Nc1ccc([N+](=O)[O-])c(C(F)(F)F)c1]

(R)-3-bromo-2-hydroxy-2-methylpropanoic acid (1.95 g, 0.0106734 mol) reacted with thionyl chloride (0.385 g, 0.0116437 mol), trimethylamine (1.276 g, 0.012614 mol), and 4-nitro-3-(trifluoromethyl)aniline (2.00 g, 0.0097031 mol) to afford the title compound. The product was purified by a silica gel column using DCM and ethyl acetate (19:1) as eluent to afford 2.70 g (75.0%) of the titled compound as yellow solid.

¹H NMR (400 MHz, DMSO-d₆) δ 10.61(s, 1 H, NH), 8.58(d, J=2.0 Hz, 1H, ArH), 8.38 (dd, J=8.8 Hz, J=2.0 Hz, 1H, ArH), 8.22 (d, J=8.8 Hz, 1H, ArH), 6.45 (br s, 1H, OH), 3.85 (d, J=10.4 Hz, 1H, CH), 3.61 (d, J=10.4 Hz, 1H, CH), 1.50 (s, 3H, CH3).

Mass (ESI, Positive): [M+H]⁺.

HRMS [C₁₁H₁₁BrF₃N₂O₄⁺]: calcd 370.9854, found 370.9854 [M+H]⁺. Purity: 95.23% (HPLC).

### (S)-3-(4-cyano-1H-pyrazol-1-yl)-2-hydroxy-2-methyl-N-(4-nitro-3-(trifluoromethyl)phenyl)propanamide (C₁₅H₁₂F₃N₅O₄) (1089)

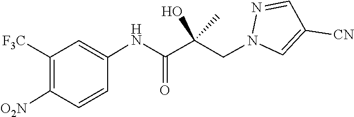

To a solution of 4-cyano-1H-pyrazole (0.376 g, 0.0040419 mol) in anhydrous THF (20mL), which was cooled in an ice water bath under an argon atmosphere, was added sodium hydride (60% dispersion in oil, 0.566 g, 0.0141466 mol). After addition, the resulting mixture was stirred for three hours. (R)-3-bromo-2-hydroxy-2-methyl-N-(4-nitro-3-(trifluoromethyl)phenyl)propanamide (1.50 g, 0.0040419 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1 to 5:1) as eluent to afford 0.52 g (33.5%) of the titled compound as yellow solid.

¹H NMR (400 MHz, DMSO-d₆) δ 10.42 (br s, 1H, NH), 8.47(d, J=2.0 Hz, 1H, ArH), 8.46 (s, 1H, Pyrazole-H), 8.30 (dd, J=8.8 Hz, J=2.0 Hz, 1H, ArH), 8.20 (d, J=8.8 Hz, 1H, ArH), 8.00 (s, 1H, Pyrazole-H), 6.44 (br s, 1H, OH), 4.55 (d, J=14.4 Hz, 1H, CH), 4.36 (d, J=14.0 Hz, 1H, CH), 1.39 (s, 3H, CH₃).

Mass (ESI, Positive): [M+H]⁺.

HRMS [C₁₅H₁₃F₃N₅O₄⁺]: calcd 384.0920, found 384.0914 [M+H]⁺. Purity: 100.00% (HPLC).

### (S)-3-(4-cyano-1H-pyrazol-1-yl)-2-hydroxy-N-(4-isothiocyanato-3-(trifluoromethyl)phenyl)-2-methylpropanamide (C₁₆H₁₃F3N₅O₂S) (1090)

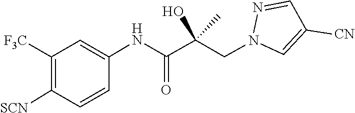

To a solution of (S)-N-(4-amino-3-(trifluoromethyl)phenyl)-3-(4-cyano-1H-pyrazol-1-yl)-2-hydroxy-2-methylpropanamide (0.135 g, 0.0003821 mol) in 5 mL of anhydrous THF, which was cooled in an ice-water bath, was added thiophosgene (88 mg, 0.0007642mo1) and triethylamine (0.193 g, 0.0019105 mol) under argon. The resulting reaction mixture was for 4-5 hours at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1) as eluent to afford 20 mg (13.3%) of the titled compound as light brown solid (not very stable).

¹H NMR (400MHz, DMSO-d₆) δ 10.13 (s, 1H, NH), 8.30 (d, J=2.0Hz, 1H, ArH), 8.13 (s, 1H, Pyrazole-H), 8.04 (d, J=8.2 Hz, 1H, ArH), 7.64 (dd, J=8.2 Hz, J=2.0 Hz, 1H, ArH), 7.45 (s, 1H, Pyrazole-H), 6.19 (s, 1H, OH), 4.39 (m, 1H, CH), 4.21 (m, 1H, CH), 1.32 (s, 3H, CH₃). Mass (ESI, Positive): [M+H]⁺. HRMS [C₁₆H₁₁F₃N₅O₂S—]: calcd 394.0586, found 396.0613 [M+H]⁺. Purity: % (HPLC).

### Preparation of Compound 1094

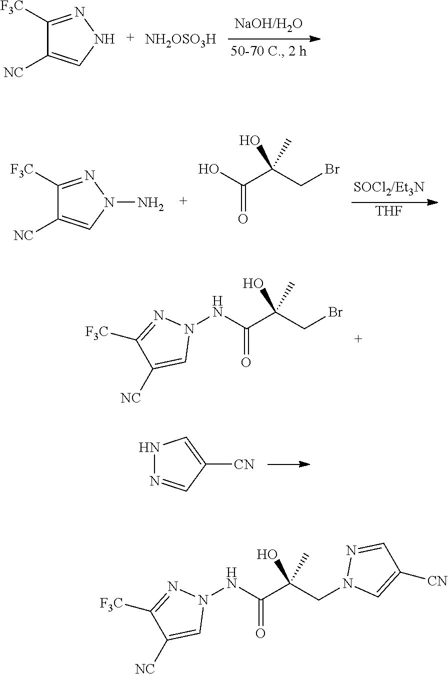

### 1-Amino-3-(Trifluoromethyl)-1H-Pyrazole-4-Carbonitrile (C₅H₃F₃N₄)

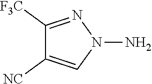 [SMILES: N#Cc1cn(N)nc1C(F)(F)F]

To a solution of 3-(trifluoromethyl)-1H-pyrazole-4-carbonitrile (0.5 g, 0.0031041 mol) in 10 mL of water was added crushed NaOH (0.5 g, 0.012416 mol). The solution was stirred at 55-60 ° C. for 20 minutes. Hydroxyamine-O-sulfonic acid (1.05 g, 0.009312 mol) was added cautiously by portion to above solution. The resulting reaction mixture was heated at 65 ° C. for 2 hours and stirred at room temperature for 2 hours. The reaction was extracted with DCM for three times. The organic layer was washed with brine, dried with MgSO₄, filtered, concentrated under vacuum, dried, and went to the next step without further purification.

¹H NMR (400 MHz, DMSO-d₆) δ

HRMS [C₅H₂F₃N₄—]: calcd 175.0232, found 175.0317 [M−H]−. Purity: % (HPLC).

### (R)-3-Bromo-N-(4-Cyano-3-(Trifluoromethyl)-1H-Pyrazol-1-Yl)-2-Hydroxy-2-Methylpropanamide (C₉H₈BrF₃N₄O₂)

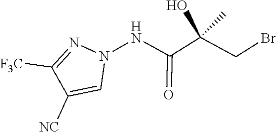 [SMILES: C[C@](O)(CBr)C(=O)Nn1cc(C#N)c(C(F)(F)F)n1]

(R)-3-bromo-2-hydroxy-2-methylpropanoic acid (0.864 g, 0.0047223 mol) reacted with thionyl chloride (0.613 g, 0.0051516 mol), trimethylamine (0.565 g, 0.0055809 mol), and 1-amino-3-(trifluoromethyl)-1H-pyrazole-4-carbonitrile (0.756 g, 0.004293 mol) to afford the title compound. The product was purified by a silica gel column using DCM and ethyl acetate (9:1 to 4:1) as eluent to afford 0.46 g (31.5%) of the titled compound as yellow solid.

¹H NMR (400 MHz, DMSO-d₆) δ Mass (ESI, Positive): [M+H]⁺.

HRMS [C₉H₇BrF₃N₄O₂—]: calcd 338.9704, found 338.9697 [M−H]−. Purity: % (HPLC).

### (S)-3-(4-Cyano-1H-Pyrazol-1-Yl)-N-(4-Cyano-3-(Trifluoromethyl)-1H-Pyrazol-1-Yl)-2-Hydroxy-2-Methylpropanamide (C₁₃H₁₀F₃N₇O₂) (1094)

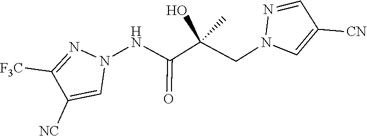 [SMILES: C[C@](O)(Cn1cc(C#N)cn1)C(=O)Nn1cc(C#N)c(C(F)(F)F)n1]

To a solution of 4-cyano-1H-pyrazole (0.15 g, 0.0016183 mol) in anhydrous THF (10 mL), which was cooled in an ice water bath under an argon atmosphere, was added sodium hydride (60% dispersion in oil, 0.19 g, 0.0047201 mol). After addition, the resulting mixture was stirred for two hours. (R)-3-bromo-N-(4-cyano-3-(trifluoromethyl)-1H-pyrazol-1-yl)-2-hydroxy-2-methylpropanamide (0.46 g, 0.0013486 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and ethyl acetate (4:1 to 2:1) as eluent to afford 0.115 g (50.4%) of the titled compound as yellow solid.

¹ NMR (400 MHz, DMSO-d₆) δ 12.27 (s, 1H, NH), 8.83 (s, 1H, Pyrazole-H), 8.46(s, 1H, Pyrazole-H), 8.15 (s, 1H, Pyrazole-H), 6.49 (s, 1H, OH), 4.51 (d, J=14.0 Hz, 1H, CH), 4.35(d, J=14.0 Hz, 1H, CH), 1.40 (s, 3H, CH3).

Mass (ESI, Positive): [M+H]⁺.

HRMS [C₁₃H₉F₃N₇O₂—]: calcd 352.0770, found 352.0761 [M−H]−. Purity: 99.00% (HPLC).

### Preparation of Compound 1092

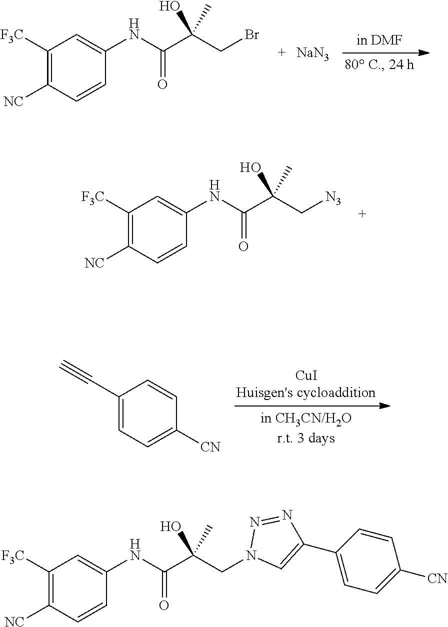

### (S)-3-Azido-N-(4-Cyano-3-(Trifluoromethyl)Phenyl)-2-Hydroxy-2-Methylpropanamide (C₁₂H₁₀F₃N₅O₂)

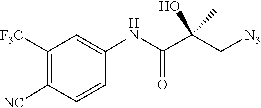

To a solution of (R)-3-bromo-N-(4-cyano-3-(trifluoromethyl)phenyl) hydroxy-2-methylpropanamide (2.00 g, 0.005696 mol) in anhydrous DMF (10 mL) was added sodium azide (0.74 g, 0.011392 mol). The resulting mixture was heated at 80 ° C. for 3-4 hours. After the end of reaction was established by TLC, the reaction was quenched by water, extracted with ethyl acetate. The organic layer was washed with brine, dried with MgSO₄, filtered, and reduced volume under vacuum. The product was purified by a silica gel column using DCM and ethyl acetate (9:1) as eluent to afford 0.93 g (52.4%) of the titled compound as yellowish solid.

¹H NMR (400 MHz, DMSO-d₆) δ 10.58 (s, 1H, NH), 8.54 (s, 1H, ArH), 8.31 (d, J=8.2 Hz, 1H, ArH), 8.11 (d, J=8.2 Hz, 1H, ArH), 6.43 (s, 1H, OH), 4.02 (d, J=14.0 Hz, 1H, CH), 3.39 (d, J=14.0 Hz, 1H, CH), 1.37 (s, 3H, CH₃).

Mass (ESI, Positive): [M+H]⁺.

HRMS [C₁₂H₁₁F₃N₅O₂⁺]: calcd 314.0865, found 314.0865 [M+H]⁺. Purity: 99.00% (HPLC).

### (S)-N-(4-cyano-3-(trifluoromethyl)phenyl)-3-(4-(4-cyanophenyl)-1H-1,2,3-triazol-1-yl)-2-hydroxy-2-methylpropanamide (C₂₁H₁₅F₃N₆O₂) (1092)

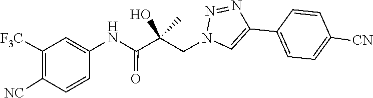 [SMILES: C[C@](O)(Cn1cc(-c2ccc(C#N)cc2)nn1)C(=O)Nc1ccc(C#N)c(C(F)(F)F)c1]

To a solution of (S)-3-azido-N-(4-cyano-3-(trifluoromethyl)phenyl)-2-hydroxy-2-methylpropanamide (0.50 g, 0.0015692 mol) in mixture of CAN and water (8 mL+2 mL) was added 4-ethynylbenzonitrile (0.30 g, 0.0023943 mol), and CuI (30 mg, 0.0001596 mol) as catalyst. The resulting mixture was stirred at room temperature for 3 days (Azide-alkyne Huisgen Cycloaddition, also called Click reaction). The reaction was quenched by water, extracted with ethyl acetate. The organic layer was washed with brine, dried with MgSO₄, filtered, and reduced volume under vacuum. The product was purified by a silica gel column using DCM and methanol (19:1) as eluent to afford 0.22 g (31%) of the titled compound as white solid.

¹H NMR (400 MHz, DMSO-d₆) δ 10.44 (s, 1H, NH), 8.63 (s, 1H, Pyrazole-H), 8.42 (s, 1H, ArH), 8.23 (d, J=8.2 Hz, 1H, ArH), 8.09 (d, J=8.2 Hz, 1H, ArH), 8.03(d, J=8.0 Hz, 2H, ArH), 7.91 (d, J=8.0 Hz, 2H, ArH), 6.56 (s, 1H, OH), 4.79 (d, J=14.0 Hz, 1H, CH), 4.61 (d, J=14.0 Hz, 1H, CH), 1.43 (s, 3H, CH₃).

Mass (ESI, Positive): [M+H]⁺.

HRMS [C₂₁H₁₆F₃N₆O₂⁺]: calcd 441.1287 found 441.1287 [M+H]⁺. Purity: % (HPLC).

### (S)-N-(4-Cyano-3-(Trifluoromethyl)Phenyl)-2-Hydroxy-2-Methyl-3-(4-(4-(Trifluoromethyl)Phenyl)-1H-1,2,3-Triazol-1-Yl)Propenamide (C₁₂H₁₅F₆N₅O₂) (1093)

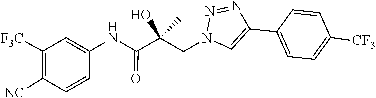 [SMILES: C[C@](O)(Cn1cc(-c2ccc(C(F)(F)F)cc2)nn1)C(=O)Nc1ccc(C#N)c(C(F)(F)F)c1]

To a solution of (S)-3-azido-N-(4-cyano-3-(trifluoromethyl)phenyl)-2-hydroxy-2-methylpropanamide (0.50 g, 0.0015692 mol) in mixture of CAN and water (8 mL+2 mL) was added 1-ethynyl-4-(trifluoromethyl)benzene (0.41 g, 0.0023943 mol), and CuI (30 mg, 0.0001596 mol) as catalyst. The resulting mixture was stirred at room temperature for 3 days (Azide-alkyne Huisgen Cycloaddition, also called Click reaction). The reaction was quenched by water, extracted with ethyl acetate. The organic layer was washed with brine, dried with MgSO₄, filtered, and reduced volume under vacuum. The product was purified by a silica gel column using hexanes and ethyl acetate (1:1 to 1:1.5) as eluent to afford 0.538 g (70%) of the titled compound as white solid.

¹H NMR (400 MHz, DMSO-d₆) δ 10.45 (s, 1H, NH), 8.59 (s, 1H, Pyrazole-H), 8.42 (s, 1H, ArH), 8.24 (d, J=8.2 Hz, 1H, ArH), 8.10 (d, J=8.2 Hz, 1H, ArH), 8.05 (d, J=8.0 Hz, 2H, ArH), 7.80 (d, J=8.0 Hz, 2H, ArH), 6.56 (s, 1H, OH), 4.80 (d, J=14.0 Hz, 1H, CH), 4.61 (d, J=14.0 Hz, 1H, CH), 1.44 (s, 3H, CH₃).

Mass (ESI, Positive): [M+H]⁺.

HRMS [C₂₁H₁₆F₃N₆O₂⁺]: calcd 441.1287 found 441.1287 [M+H]⁺. Purity: % (HPLC).

### Example 2

### Androgen Receptor Binding, Transactivation, Degradation, and Metabolism of SARDs

### Ligand Binding Assay (Ki Values):

Objective: To determine SARDs binding affinity to the AR-LBD.

Method: hAR-LBD (633-919) was cloned into pGex4t.1. Large scale GST-tagged AR-LBD was prepared and purified using a GST column. Recombinant AR-LBD was combined with [³H]mibolerone (PerkinElmer, Waltham, MA) in buffer A (10 mM Tris, pH 7.4, 1.5 mM disodium EDTA, 0.25 M sucrose, 10 mM sodium molybdate, 1 mM PMSF) to determine the equilibrium dissociation constant (Kd) of [³H]mibolerone. Protein was incubated with increasing concentrations of [³H]mibolerone with and without a high concentration of unlabeled mibolerone at 4° C. for 18 h in order to determine total and non-specific binding. Non-specific binding was then subtracted from total binding to determine specific binding and non-linear regression for ligand binding curve with one site saturation to determine the Kd of mibolerone.

Increasing concentrations of SARDs or DHT (range: 10⁻¹² to 10⁻² M) were incubated with [³H]mibolerone and AR LBD using the conditions described above. Following incubation, the ligand bound AR-LBD complex was isolated using Bio Gel HT® hydroxyapatite, washed and counted in a scintillation counter after adding scintillation cocktail. Values are expressed as Ki.

Transactivation Assay with wt AR (IC₅₀ Values)

Objective: To determine the effect of SARDs on androgen-induced transactivation of AR wildtype (wt).

Method: HEK-293 cells were plated at 125,000 cells/well of a 24 well plate in DME+5% csFBS without phenol red. Cells were transfected with 0.25 ug GRE-LUC, 10 ng CMV-renilla LUC, and 50 ng CMV-hAR(wt) using Lipofectamine transfection reagent in optiMEM medium. Medium was changed 24 h after transfection to DME+5% csFBS without phenol red and treated with a dose response of various drugs (1 pM to 10 μM). SARDs and antagonists were treated in combination with 0.1 nM R1881. Luciferase assay was performed 24 h after treatment on a Biotek synergy 4 plate reader. Firefly luciferase values were normalized to renilla luciferase values.

### Plasmid Constructs and Transient Transfection

Human AR cloned into CMV vector backbone was used for the transactivation study. HEK-293 cells were plated at 120,000 cells per well of a 24 well plate in DME+5% csFBS. The cells were transfected using Lipofectamine (Invitrogen, Carlsbad, CA) with 0.25 mg GRE-LUC, 0.01 mg CMV-LUC (renilla luciferase) and 25 ng of the AR. The cells were treated 24 hrs after transfection as indicated in the figures and the luciferase assay performed 48 hrs after transfection. Data are represented as IC₅₀ obtained from four parameter logistics curve.

### LNCaP Gene Expression Assay

Method: LNCaP cells were plated at 15,000 cells/well of a 96 well plate in RPMI+1% csFBS without phenol red. Forty-eight hours after plating, cells were treated with a dose response of SARDs. Twenty four hours after treatment, RNA was isolated using cells-to-ct reagent, cDNA synthesized, and expression of various genes was measured by realtime rtPCR (ABI 7900) using taqman primers and probes. Gene expression results were normalized to GAPDH.

### LNCaP Growth Assay

Method: LNCaP cells were plated at 10,000 cells/well of a 96 well plate in RPMI+1% csFBS without phenol red. Cells were treated with a dose response of SARDs. Three days after treatment, cells were treated again. Six days after treatment, cells were fixed and cell viability was measured by SRB assay.

### LNCaP or AD1 Degradation (AR FL)

Method: LNCaP or AD1 cells expressing full length AR were plated at 750,000-1,000,000 cells/well of a 6 well plate in growth medium (RPMI+10% FBS). Twenty four hours after plating, medium was changed to RPMI+1% csFBS without phenol red and maintained in this medium for 2 days. Medium was again changed to RPMI+1% csFBS without phenol red and cells were treated with SARDs (1 nM to 10 μM) in combination with 0.1 nM R1881. After 24 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

### 22RV1 and D567es Degradation (AR SV)

Method: 22RV1 and D567es cells expressing AR splice variants were plated at 750,000-1,000,000 cells/well of a 6 well plate in growth medium (RPMI+10% FBS). Twenty four hours after plating, medium was changed and treated. After 24-30 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

### 22RV1 Growth and Gene Expression

Methods: Cell growth was evaluated as described before by SRB assay. Cells were plated in a 96 well plate in full serum and treated for 6 days with medium change after day 3. Gene expression studies were performed in 22RV1 cells plated in 96 well plate at 10,000 cells/well in RPMI+10% FBS. Twenty four hours after plating, cells were treated for 3 days and gene expression studies were performed as described before.

Transactivation (IC₅₀): in vitro AR antagonism of indicated compounds in Table 1. COS7 cells were transfected with 0.25 ug GRE-LUC, 0.01 ug CMV-renilla LUC, and 25 ng CMV-hAR using lipofectamine in optiMEM medium. Cells were treated 24 hours after transfection in the presence of 0.1 nM R1881 and luciferase assay performed 48 hours after transfection. Firefly luciferase values were normalized to renilla luciferase values.

Degradation: Table 1 presents FL and SV AR degradation activity for indicated compounds. The numbers under each column represents the % change from vehicle. The bands were quantified using Image software. For each value, the AR band was divided by GAPDH band and the % difference from vehicle was calculated and represented. The numbers shown are 0 (no degradation) or represented as decreases in AR levels normalized for GAPDH levels. For FL AR degradation, LNCaP cells were maintained in charcoal-stripped FBS-containing medium for 2 days. Cells were treated in this medium in the presence of 0.1 nM R1881. Cells were harvested 24 hours after treatment, protein extracted, and Western blot for AR and GAPDH was performed. For SV AR degradation, 22RV1 cells were treated as indicated for LNCaP.

### Determination of Metabolic Stability (In Vitro CLᵢₙₜ) of Test Compounds Phase I Metabolism

The assay was done in a final volume of 0.5 ml in duplicates (n=2). Test compound (1 μM) was pre-incubated for 10 minutes at 37° C. in 100 mM Tris-HCl, pH 7.5 containing 0.5 mg/ml liver microsomal protein. After pre-incubation, reaction was started by addition of 1 mM NADPH (pre-incubated at 37° C.). Incubations were carried out in triplicate and at various time-points (0, 5, 10, 15, 30 and 60 minutes) 100 μL aliquots were removed and quenched with 100 μL of acetonitrile containing internal standard. Samples were vortex mixed and centrifuged at 4000 rpm for 10 minutes. The supernatants were transferred to 96 well plates and submitted for LC-MS/MS analysis. As control, sample incubations done in absence of NADPH were included. From %PCR (% Parent Compound Remaining), rate of compound disappearance is determined (slope) and in vitro CLint (μL/min/mg protein) was calculated.

### Metabolic Stability in Phase I & Phase II Pathways

In this assay, test compound was incubated with liver microsomes and disappearance of drug was determined using discovery grade LC-MS/MS. To stimulate Phase II metabolic pathway (glucuronidation), UDPGA and alamethicin was included in the assay. LC-MS/MS analysis

The analysis of the compounds under investigation was performed using LC-MS/MS system consisting of Agilent 1100 HPLC with an MDS/Sciex 4000 Q-Trap™ mass spectrometer. The separation was achieved using a C18 analytical column (Alltima™, 2.1×100 mm, 3 μm) protected by a C18 guard cartridge system (SecurityGuard™ ULTRA Cartridges UHPLC for 4.6 mm ID columns, Phenomenex). Mobile phase was consisting of channel A (95% acetonitrile+5% water+0.1% formic acid) and channel C (95% water+5% acetonitrile+0.1% formic acid) and was delivered at a flow rate of 0.4 mL/min. The volume ratio of acetonitrile and water was optimized for each of the analytes. Multiple reaction monitoring (MRM) scans were made with curtain gas, collision gas, nebulizer gas, and auxiliary gas optimized for each compound, and source temperature at 550° C. Molecular ions were formed using an ion spray voltage of −4200 V (negative mode). Declustering potential, entrance potential, collision energy, product ion mass, and cell exit potential were optimized for each compound.

### LC-MS/MS Analysis for Determining Rat Serum Concentrations

Serum was collected 24-30 hours after last dose. 100 μL of serum was mixed with 200 μL of acetonitrile/internal standard. Standard curves were prepared by serial dilution of standards in nM with 100 μL of rat serum, concentrations were 1000, 500, 250, 125, 62.5, 31.2, 15.6, 7.8, 3.9, 1.9, 0.97, and 0. Standards were with extracted with 200 μL of acetonitrile/internal standard. The internal standard for these experiments was (S)-3-(4-cyanophenoxy)-N-(3-(chloro)-4-cyanophenyl)-2-hydroxy-2-methylpropanamide.

The instrumental analysis of the analyte SARD was performed using LC-MS/MS system consisting of Agilent 1100 HPLC with an MDS/Sciex 4000 Q-Trap™ mass spectrometer. The separation was achieved using a C18 analytical column (Alltima™, 2.1×100 mm, 3 μm) protected by a C18 guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 mL/min at 70% A and 30% B. The total runtime for analyte SARD was optimized but generally 2-4 minutes, and the volume injected was 10 μL. Multiple reaction monitoring (MRM) scans were made with curtain gas at 10; collision gas at medium; nebulizer gas at 60.0 and auxiliary gas at 60.0 and source temperature at 550° C. Molecular ions were formed using an ion spray voltage (IS) of 4200 (negative mode). Declustering potential (DP), entrance potential (EP), collision energy (CE), product ion mass, and cell exit potential (CXP) were optimized for each analyte SARD for the mass pair observed.

### Log P: Octanol-Water Partition Coefficient (Log P)

Log P is the log of the octanol-water partition coefficient, commonly used early in drug discovery efforts as a rough estimate of whether a particular molecule is likely to cross biological membranes. Log P was calculated using ChemDraw Ultra version is 12.0.2.1016 (Perkin-Elmer, Waltham, Mass. 02451). Calculated Log P values are reported in Table 1 in the column labeled ‘Log P (−0.4 to +5.6)’. Lipinski's rule of five is a set of criteria intended to predict oral bioavailability. One of these criteria for oral bioavailability is that the Log P is between the values shown in the column heading (−0.4 (relatively hydrophilic) to +5.6 (relatively lipophilic) range), or more generally stated <5. One of the goals of SARD design was to improve water solubility. The monocyclic templates of this invention such as the pyrazoles, pyrroles, etc. were more water soluble than earlier analogs.

TABLE 1
In vitro screening of LBD binding (Ki), AR antagonism (IC50), SARD activity, and metabolic stability

| | | | | | | SARD Activity | | DMPK |
|---|---|---|---|---|---|---|---|---|
| | | | | | | | S.V. | (MLM) |
| | | | | Binding/Wt. | | Full Length % | (22RV1) % | T_{1/2} (min) |
| Compd ID | | Log P | | Kᵢ ( [symbol] M) | IC₅₀ | degradation at | degradation | Clᵢₙₜ (μL/ |
| (Scaffold) | Structure | (−0.4 to +5.6) | M.W. | (DHT = 1 [symbol] M) | ( [symbol] M) | 1.10 μM | at 10 μM | min/mg) |
| Enzalut- amide | 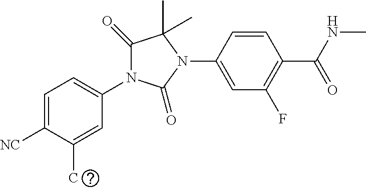 [SMILES: CNC(=O)c1ccc(N2C(=O)N(c3ccc(C#N)c(C)c3)C(=O)C2(C)C)cc1F] | 4.56 | 464.44 | 3641.29 | 216.3 | | | |
| Eno- bosarm | 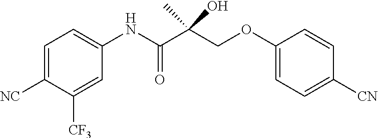 [SMILES: C[C@](O)(COc1ccc(C#N)cc1)C(=O)Nc1ccc(C#N)c(C(F)(F)F)c1] | 3.44 | 389.89 | 20.21 | −20 | | | |
| R-Bicut- amide | 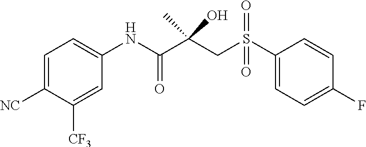 [SMILES: C[C@](O)(CS(=O)(=O)c1ccc(F)cc1)C(=O)Nc1ccc(C#N)c(C(F)(F)F)c1] | 2.57 | 430.37 | 508.84 | 248.2 | | | |
| Enzalut- amide | 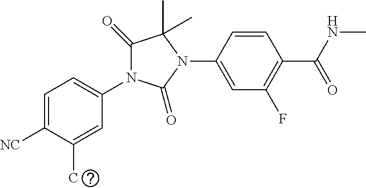 [SMILES: CNC(=O)c1ccc(N2C(=O)N(c3ccc(C#N)c(C)c3)C(=O)C2(C)C)cc1F] | 4.56 | 464.44 | 3641.29 | 216.3 | | | |
| ARN- 509 | 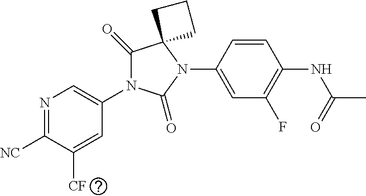 | 3.47 | 477.43 | 1452.29 | | 0 | 0 | |
| 11 | 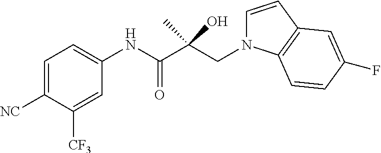 [SMILES: C[C@](O)(Cn1ccc2cc(F)ccc21)C(=O)Nc1ccc(C#N)c(C(F)(F)F)c1] | 3.47 | 405.35 | 267.39 | 85.10 | 65-83 | 60-100 | 12.35 56.14 |
| 15 | 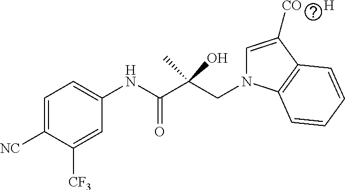 | 2.87 | 431.36 | >10,000 | — | | | 29.79 23.28 |
| 30 | 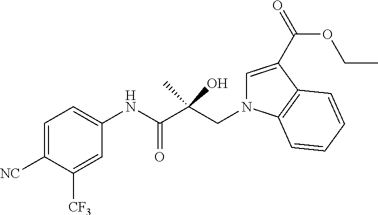 [SMILES: CCOC(=O)c1cn(C[C@](C)(O)C(=O)Nc2ccc(C#N)c(C(F)(F)F)c2)c2ccccc12] | 3.47 | 459.42 | 995.23 | 971.78 | | | 25.78 26.89 |
| 31 | 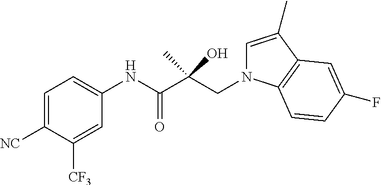 [SMILES: Cc1cn(C[C@](C)(O)C(=O)Nc2ccc(C#N)c(C(F)(F)F)c2)c2ccc(F)cc12] | 3.95 | 419.37 | 547.27 | 157.41 | | | 21.77 31.84 |
| 1002 | 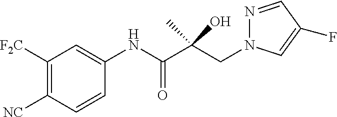 | 203 | 336.27 | No binding | 199.36 | 100 | 100 | 77.96 0.89 |
| 1024 | 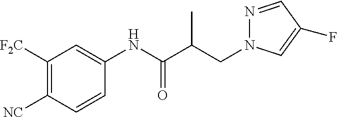 | 2.86 | 340.25 | No binding | 463.9 | 60 | 70 | Infnity 0 |
| 1022 | 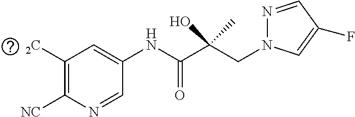 | 1.11 | 357.26 | No binding | 62.2 | 54 | 31 | |
| 1048 | 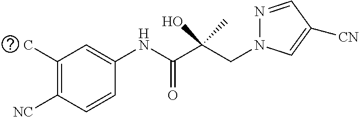 [SMILES: Cc1cc(NC(=O)[C@@](C)(O)Cn2cc(C#N)cn2)ccc1C#N] | 1.90 | 363.29 | 1499 | 44.5 | 90 | 100 | |
| 44 | 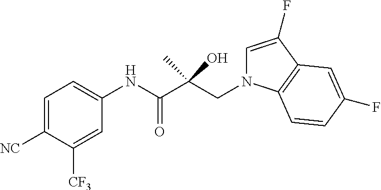 [SMILES: C[C@](O)(Cn1cc(F)c2cc(F)ccc21)C(=O)Nc1ccc(C#N)c(C(F)(F)F)c1] | 3.63 | 423.34 | 317.64 | 274.3 | 72 | 84 | 7.7 |
| 45 | 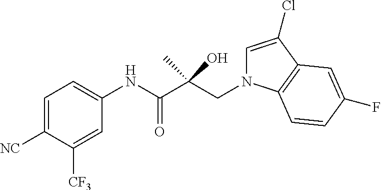 [SMILES: C[C@](O)(Cn1cc(Cl)c2cc(F)ccc21)C(=O)Nc1ccc(C#N)c(C(F)(F)F)c1] | 4.03 | 439.79 | 754.7 | 366.9 | 60 | 80 | 23.44 |
| 47 | 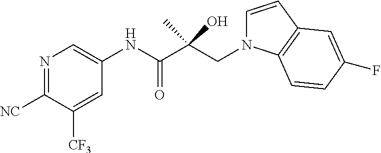 [SMILES: C[C@](O)(Cn1ccc2cc(F)ccc21)C(=O)Nc1cnc(C#N)c(C(F)(F)F)c1] | 2.55 | 406.33 | 757.29032 | 20.68 nM | 57, 97 | | |
| 1065 | 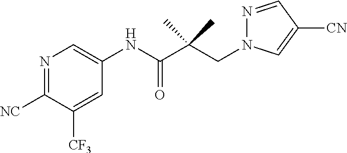 | 0.99 | 451.63 | 89.34 | 59.4 | 17, +15 | | |
| 1072 | 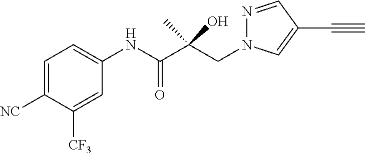 [SMILES: C#Cc1cnn(C[C@](C)(O)C(=O)Nc2ccc(C#N)c(C(F)(F)F)c2)c1] | 2.03 | 362.31 | No binding | 276 | 7, 28 | | |
| 1073 | 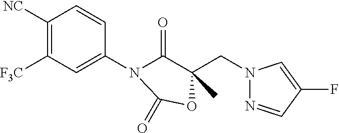 [SMILES: C[C@@]1(Cn2cc(F)cn2)OC(=O)N(c2ccc(C#N)c(C(F)(F)F)c2)C1=O] | 2.83 | 382.27 | | 1131 | 18, 50 | | |
| 1074 | 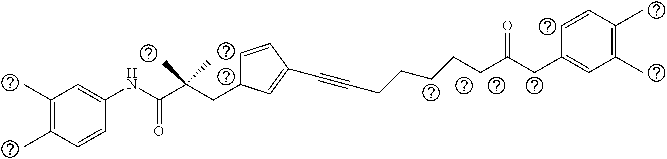 | 3.7 | 676.57 | — | 420.8 | 48, 98 | | |
| 1075 | 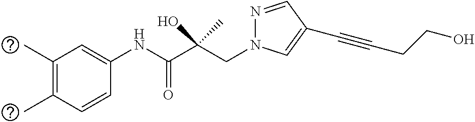 [SMILES: Cc1ccc(NC(=O)[C@@](C)(O)Cn2cc(C#CCCO)cn2)cc1C] | 1.84 | 406.36 | — | 2243 | 51, 80 | | |
| 1076 | 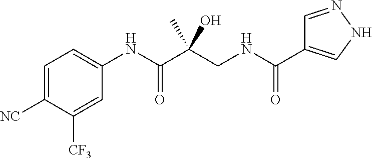 [SMILES: C[C@](O)(CNC(=O)c1cn[nH]c1)C(=O)Nc1ccc(C#N)c(C(F)(F)F)c1] | 0.77 | 381.31 | | Agonist | 16 | 60 | |
| 48 | 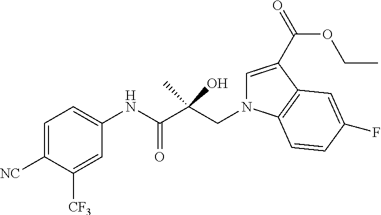 [SMILES: CCOC(=O)c1cn(C[C@](C)(O)C(=O)Nc2ccc(C#N)c(C(F)(F)F)c2)c2ccc(F)cc12] | 3.63 | 477.41 | — | 197.7 | 0 | | |
| 49 | 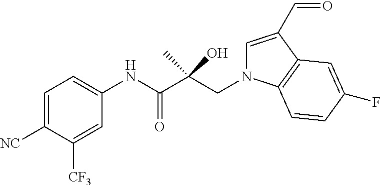 [SMILES: C[C@](O)(Cn1cc(C=O)c2cc(F)ccc21)C(=O)Nc1ccc(C#N)c(C(F)(F)F)c1] | 3.21 | 433.36 | — | 122.8 | 72 | — | 36 19.06 |
| 1078 | 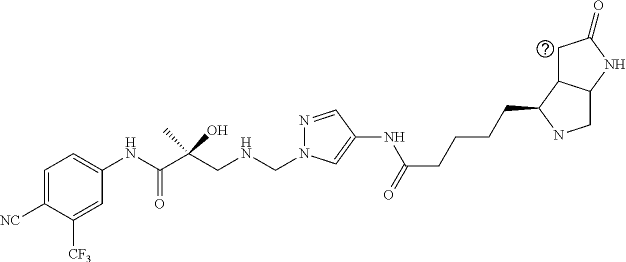 [SMILES: C[C@](O)(CNCn1cc(NC(=O)CCCC[C@@H]2NCC3NC(=O)CC32)cn1)C(=O)Nc1ccc(C#N)c(C(F)(F)F)c1] | 0.96 | 579.59 | | No effect | | | |
| 50 | 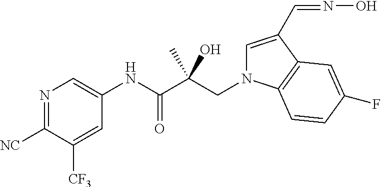 [SMILES: C[C@](O)(Cn1cc(C=NO)c2cc(F)ccc21)C(=O)Nc1cnc(C#N)c(C(F)(F)F)c1] | 3.6 | 448.37 | No binding | 58.25 | 78 | 56 | Infnity 0.000 |
| 1079 | 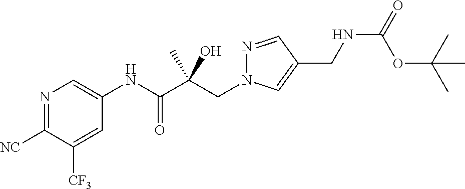 [SMILES: CC(C)(C)OC(=O)NCc1cnn(C[C@](C)(O)C(=O)Nc2cnc(C#N)c(C(F)(F)F)c2)c1] | 1.39 | 468.43 | | No effect | | | |
| 1080 | 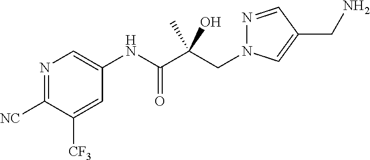 [SMILES: C[C@](O)(Cn1cc(CN)cn1)C(=O)Nc1cnc(C#N)c(C(F)(F)F)c1] | 0 | 368.31 | | Agonist | | | |
| 1082 | 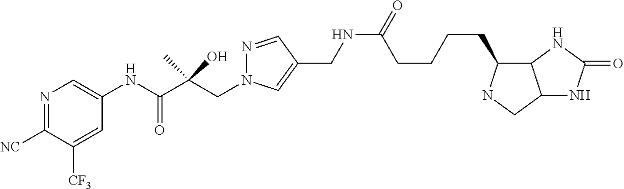 [SMILES: C[C@](O)(Cn1cc(CNC(=O)CCCC[C@@H]2NCC3NC(=O)NC32)cn1)C(=O)Nc1cnc(C#N)c(C(F)(F)F)c1] | 0.12 | 594.61 | | 766 | | | |
| 51 | 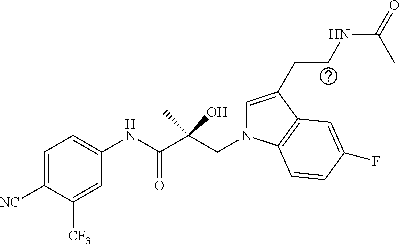 [SMILES: CC(=O)NCCc1cn(C[C@](C)(O)C(=O)Nc2ccc(C#N)c(C(F)(F)F)c2)c2ccc(F)cc12] | 2.90 | 489.42 | | 3434 | | | |
| 53 | 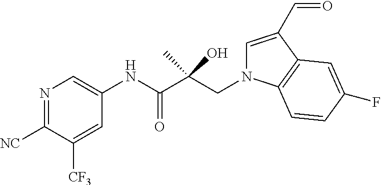 [SMILES: C[C@](O)(Cn1cc(C=O)c2cc(F)ccc21)C(=O)Nc1cnc(C#N)c(C(F)(F)F)c1] | 2.30 | 434.34 | No binding | 746 | 0, 83 | | |
| 1083 | 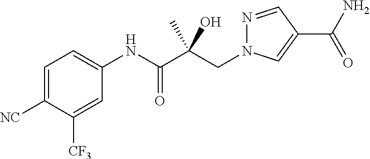 [SMILES: C[C@](O)(Cn1cc(C(N)=O)cn1)C(=O)Nc1ccc(C#N)c(C(F)(F)F)c1] | 0.77 | 381.31 | | — | | | |
| 54 | 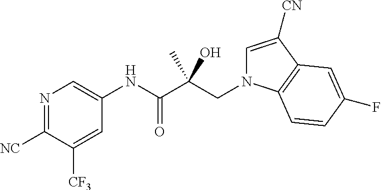 [SMILES: C[C@](O)(Cn1cc(C#N)c2cc(F)ccc21)C(=O)Nc1cnc(C#N)c(C(F)(F)F)c1] | 3.50 | 430.36 | No binding | 160 | 67 | 45 | 17.12 12.63 |
| 55 | 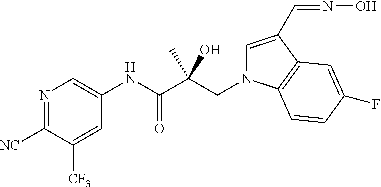 [SMILES: C[C@](O)(Cn1cc(C=NO)c2cc(F)ccc21)C(=O)Nc1cnc(C#N)c(C(F)(F)F)c1] | 2.69 | 449.36 | No binding | 133.5 | 0 | 15 | 45 50.15 |
| 56 | 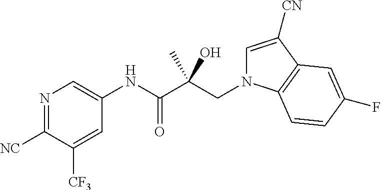 [SMILES: C[C@](O)(Cn1cc(C#N)c2cc(F)ccc21)C(=O)Nc1cnc(C#N)c(C(F)(F)F)c1] | 2.59 | 431.34 | 644 | 487.8 | 0, 70 | 25 | |
| 57 | 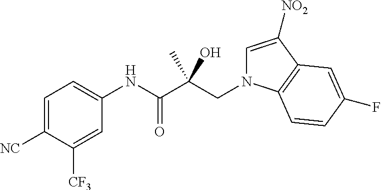 [SMILES: C[C@](O)(Cn1cc([N+](=O)[O-])c2cc(F)ccc21)C(=O)Nc1ccc(C#N)c(C(F)(F)F)c1] | 3.02 | 450.34 | 424 | 62.3 | | | |
| 58 | 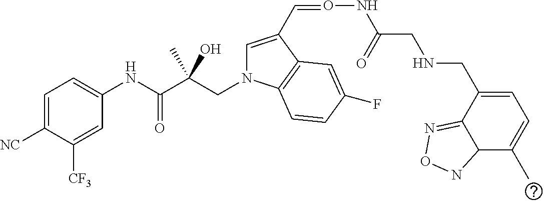 | | 681.55 | | | | | |
| 99A | 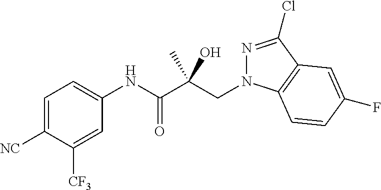 [SMILES: C[C@](O)(Cn1nc(Cl)c2cc(F)ccc21)C(=O)Nc1ccc(C#N)c(C(F)(F)F)c1] | 3.98 | 440.78 | 1342.9 | 165 | | | 34, 23 |
| 99B | 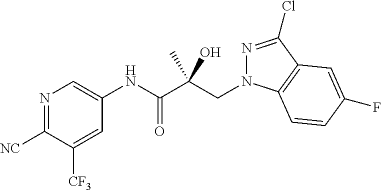 [SMILES: C[C@](O)(Cn1nc(Cl)c2cc(F)ccc21)C(=O)Nc1cnc(C#N)c(C(F)(F)F)c1] | 3.07 | 441.77 | 3006.3 | 183 | | | Infinity, 49 |
| 99C | 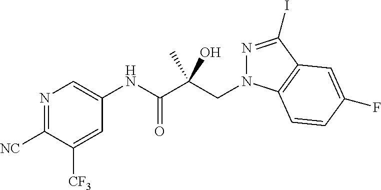 [SMILES: C[C@](O)(Cn1nc(I)c2cc(F)ccc21)C(=O)Nc1cnc(C#N)c(C(F)(F)F)c1] | 3.4 | 533.22 | 3268 | 236.7 | | | |
| 99D | 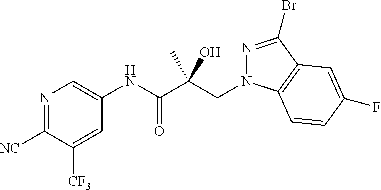 [SMILES: C[C@](O)(Cn1nc(Br)c2cc(F)ccc21)C(=O)Nc1cnc(C#N)c(C(F)(F)F)c1] | 3.4 | 486.22 | | 162 | | | |
| 99E | 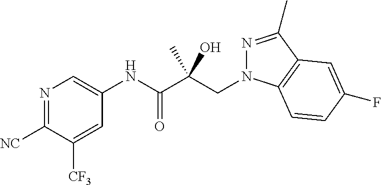 [SMILES: Cc1nn(C[C@](C)(O)C(=O)Nc2cnc(C#N)c(C(F)(F)F)c2)c2ccc(F)cc12] | 2.87 | 421.12 | | 89.1 | | | |
| 1084 | 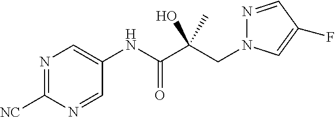 [SMILES: C[C@](O)(Cn1cc(F)cn1)C(=O)Nc1cnc(C#N)nc1] | 0.06 | 290.25 | | — | | | |
| 1085 | 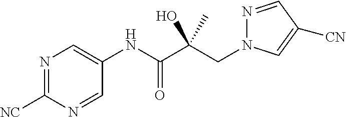 [SMILES: C[C@](O)(Cn1cc(C#N)cn1)C(=O)Nc1cnc(C#N)nc1] | 0.06 | 297.27 | | — | | | |
| 120 | 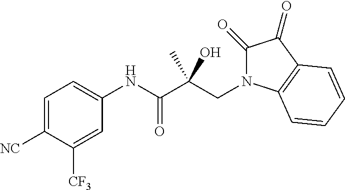 [SMILES: C[C@](O)(CN1C(=O)C(=O)c2ccccc21)C(=O)Nc1ccc(C#N)c(C(F)(F)F)c1] | 2.07 | 417.34 | | — | | | |
| 1086 | 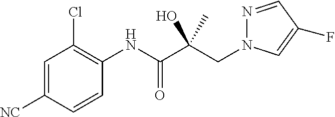 [SMILES: C[C@](O)(Cn1cc(F)cn1)C(=O)Nc1ccc(C#N)cc1Cl] | 1.66 | 322.72 | | 1232 | | | |
| 1087 | 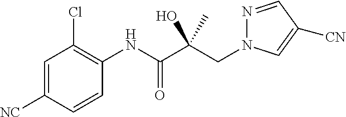 [SMILES: C[C@](O)(Cn1cc(C#N)cn1)C(=O)Nc1ccc(C#N)cc1Cl] | 1.54 | 329.74 | | 1123 | | | |
| 99F | 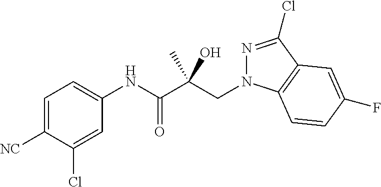 [SMILES: C[C@](O)(Cn1nc(Cl)c2cc(F)ccc21)C(=O)Nc1ccc(C#N)c(Cl)c1] | 3.62 | 407.23 | No binding | 560 | 20, 22 | | 27.4 2.5 |
| 1088 | 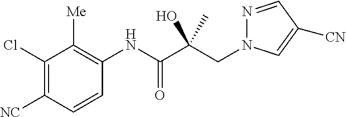 [SMILES: Cc1c(NC(=O)[C@@](C)(O)Cn2cc(C#N)cn2)ccc(C#N)c1Cl] | 2.03 | 343.77 | | 1700 | | | |
| 1089 | 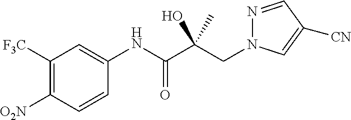 [SMILES: C[C@](O)(Cn1cc(C#N)cn1)C(=O)Nc1ccc([N+](=O)[O-])c(C(F)(F)F)c1] | 1.90 | 383.28 | | 29.2 | 15 | | |
| 99G | 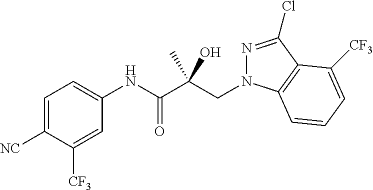 [SMILES: C[C@](O)(Cn1nc(Cl)c2c(C(F)(F)F)cccc21)C(=O)Nc1ccc(C#N)c(C(F)(F)F)c1] | 4.74 | 490.79 | No binding | 48.5 | 11, 20 | | 105 0.66 |
| 99H | 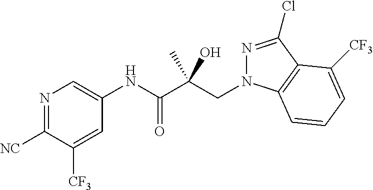 [SMILES: C[C@](O)(Cn1nc(Cl)c2c(C(F)(F)F)cccc21)C(=O)Nc1cnc(C#N)c(C(F)(F)F)c1] | 3.83 | 491.77 | — | 20.97 | −4 | | 120.6 0.6 |
| 1090 | 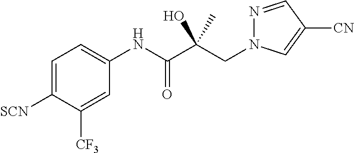 | 2.03 | 395.36 | | — | | | |
| 1091 | 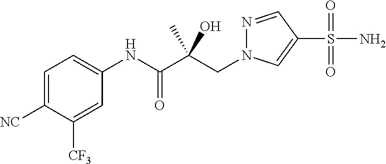 [SMILES: C[C@](O)(Cn1cc(S(N)(=O)=O)cn1)C(=O)Nc1ccc(C#N)c(C(F)(F)F)c1] | 0.61 | 417.36 | | 11.500 | 22 | | |
| 1092 | 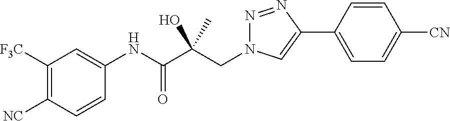 [SMILES: C[C@](O)(Cn1cc(-c2ccc(C#N)cc2)nn1)C(=O)Nc1ccc(C#N)c(C(F)(F)F)c1] | 3.61 | 440.38 | | 62 | | | |
| 1093 | 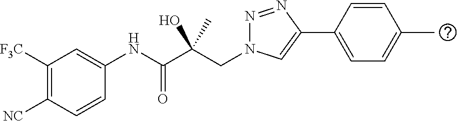 [SMILES: Cc1ccc(-c2cn(C[C@](C)(O)C(=O)Nc3ccc(C#N)c(C(F)(F)F)c3)nn2)cc1] | 4.50 | 483.37 | | 214 | | | |
| 1094 | 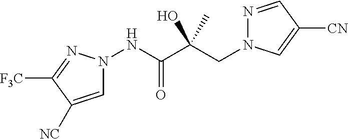 [SMILES: C[C@](O)(Cn1cc(C#N)cn1)C(=O)Nn1cc(C#N)c(C(F)(F)F)n1] | | 353.26 | | — | | | |

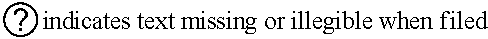

[symbol]

indicates data missing or illegible when filed

### Example 3: AR Inhibition Efficacy

The compounds of the present invention exhibited unexpected improved AR inhibition efficacy. AR inhibition data was collected as described in Example 2 above for ‘Transactivation Assay with wt AR (IC₅₀ values)’. IC₅₀ values will vary as experiments are repeated but rank ordering does not change and enzalutamide was run as a standard agent in all experiments so that relative potencies can be compared. Representative values are shown in Table 1 above and may differ slightly from the individual experiment values shown in some of the figures called out below.

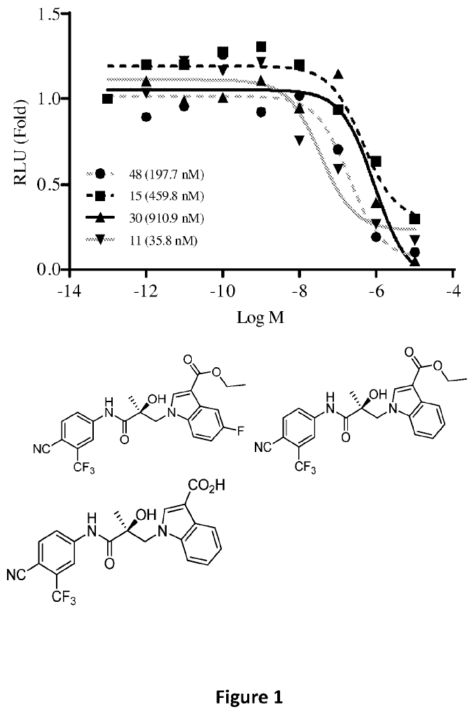

As shown in FIG. 1, compound 48 exhibited 2- to 4-fold unexpectedly improved AR inhibition potency compared to 30 and 15. This suggests that the addition of the 5-fluoro group to 30 or conversion of the 3-COOH of 15 to 3-COOEt significantly improves the wtAR inhibition potency.

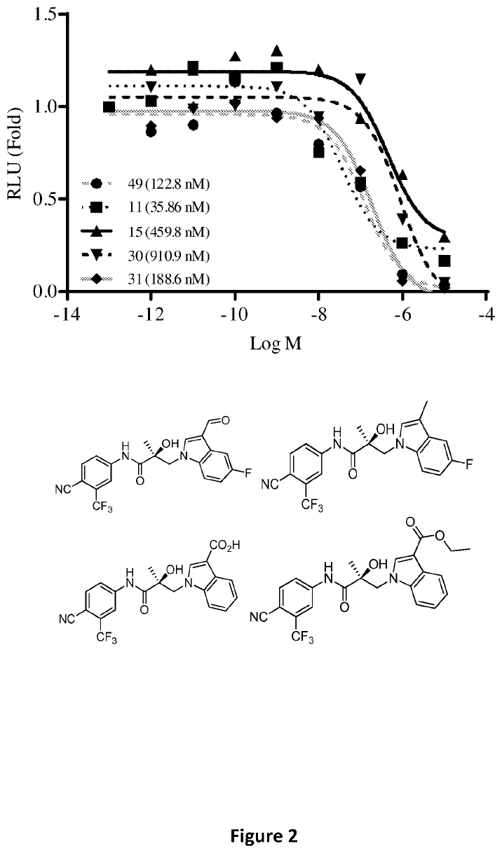

FIG. 2 demonstrates that the potency of 49 (3-formyl) is improved for all but 11. Additionally, the introduction of 3-formyl group into 49 unexpectedly improved metabolic stability as reflected by the 2 to 4-fold longer T_{1/2} in mouse liver microsomes (MLM) compared to close structural analogs 31 (3-CH₃), 11 (3-H), 30 (3-COOEt), and 15 (3-COOH) (Table 2). The combination of retained potency and improved metabolic stability is expected to improve the ability of 49 to exert AR antagonism in vivo.

TABLE 2
Metabolic Stability In Mouse Liver Microsomes

MLM

| Cmpd. | T_{1/2} | Clᵢₙₜ |
|---|---|---|
| 49 | 54.96 | 12.61 |
| 11 | 12.11 | 57.58 |
| 15 | 29.78 | 23.28 |
| 30 | 25.78 | 26.39 |
| 31 | 21.77 | 31.84 |

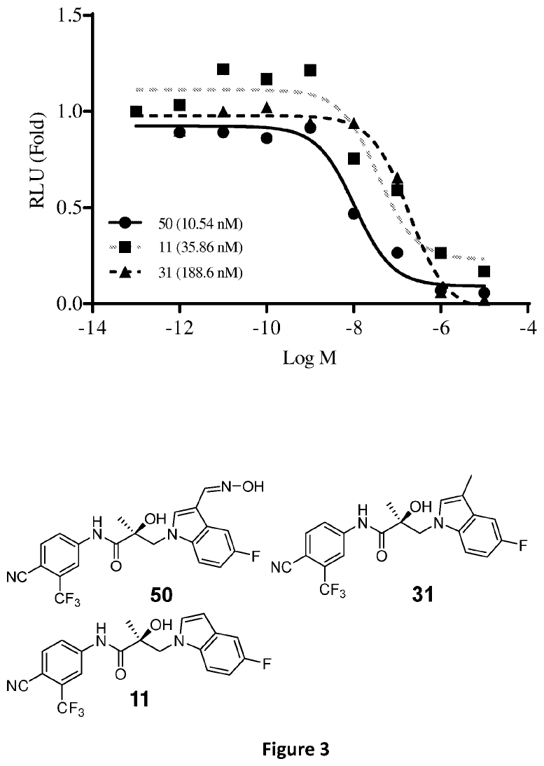

The introduction of the oxime (—CH═N—OH) at the 3-position of 11 (3-H) or 31 (3-methyl) to produce 50 unexpectedly enhanced potency of wtAR inhibition by by 3- and 15-fold, respectively (FIG. 3).

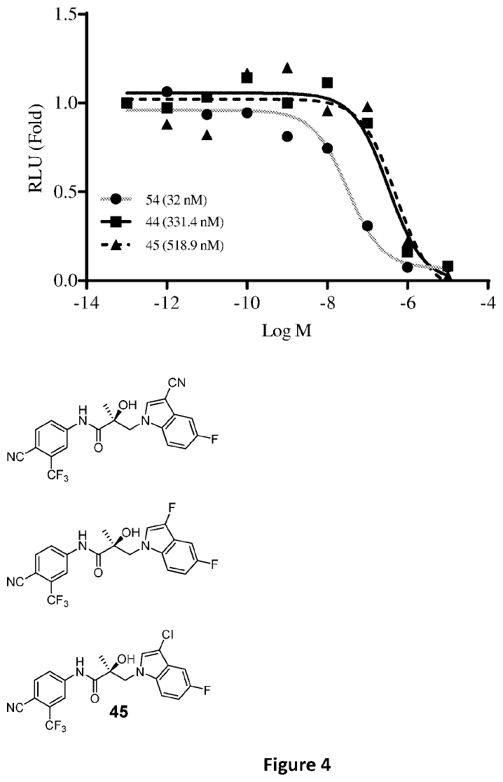

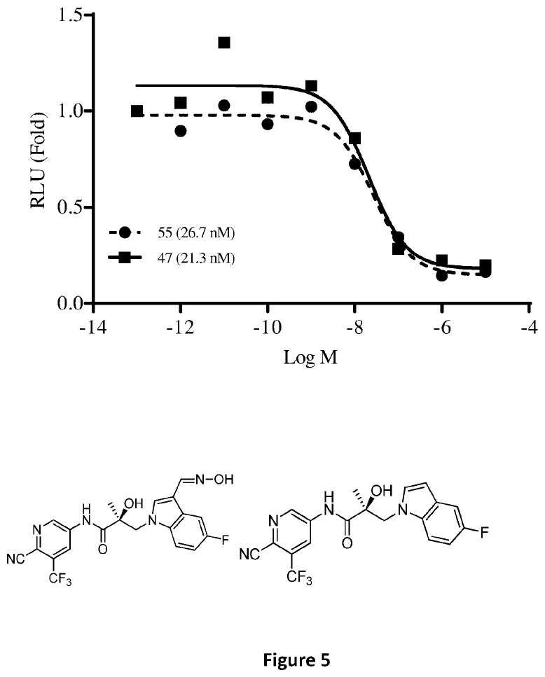

FIG. 4 demonstrates, unexpectedly, that replacement of the 3-F (44) and 3-Cl (45) groups with a 3-CN (54) enhanced the in vitro efficacy by 10- and 15-fold, respectively. Additionally, the introduction of the 3-oxime into 47 (3-H) to produce 55 was tolerated, producing equipotent in vitro potency (FIG. 5), but the 3-oxime is known to increase MLM and RLM stabilities for indole SARDs (see data for 50 above and below).

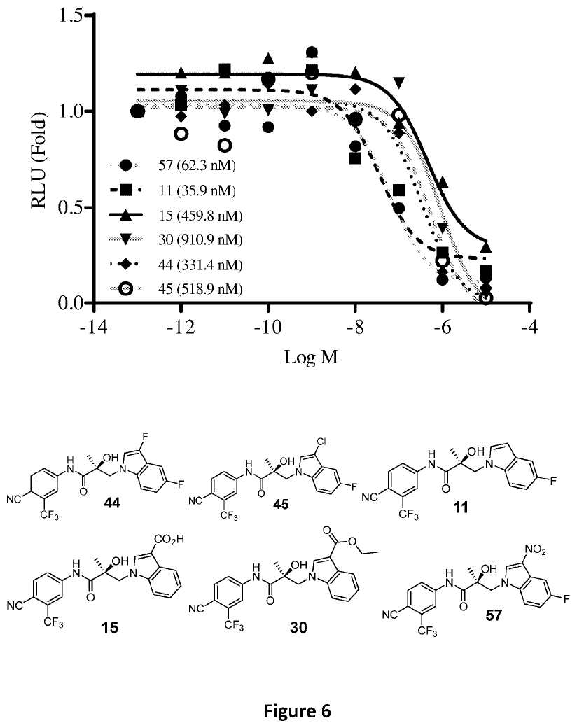

The replacement of the 3-COOH (15), 3-F (44), 3-Cl (45), and 3-COOEt (30) groups with a 3-NO2 (57) increased in vitro efficacy by at least 5-fold (FIG. 6).

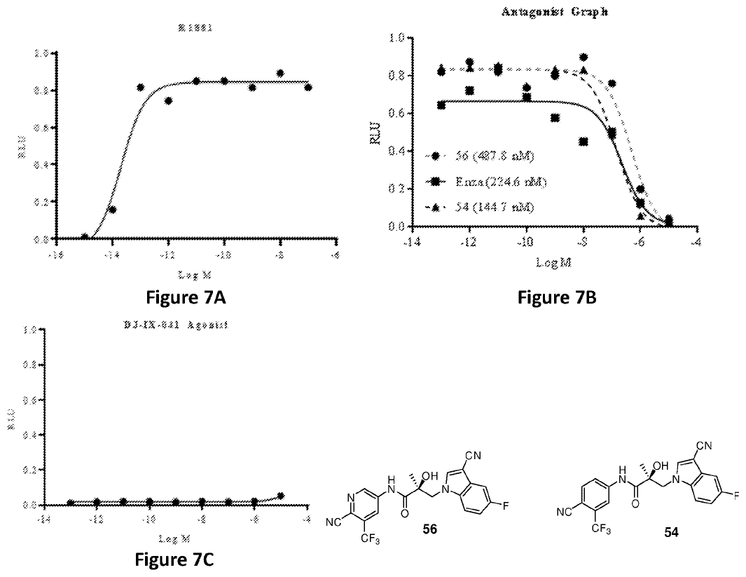

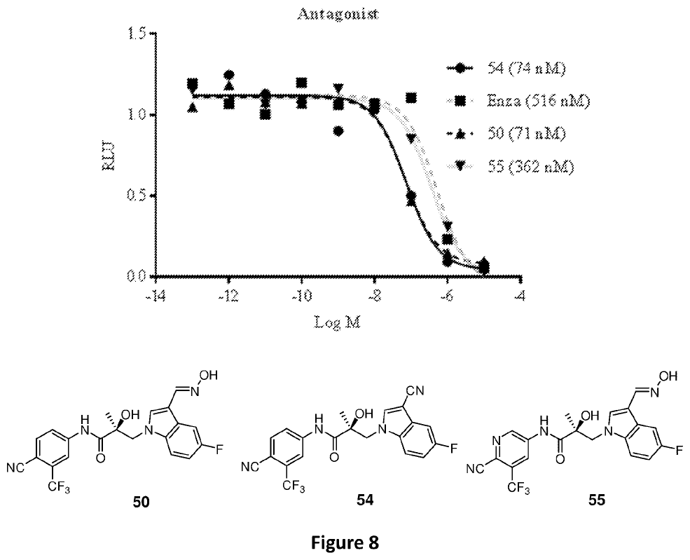

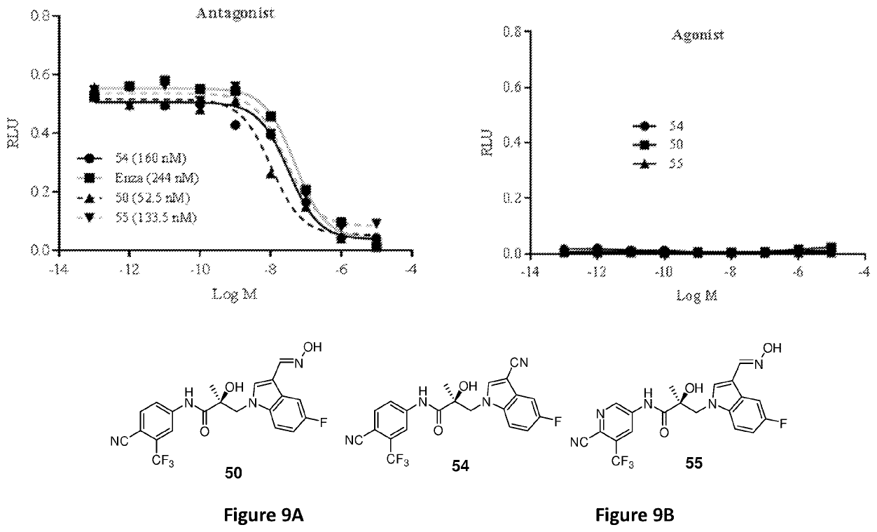

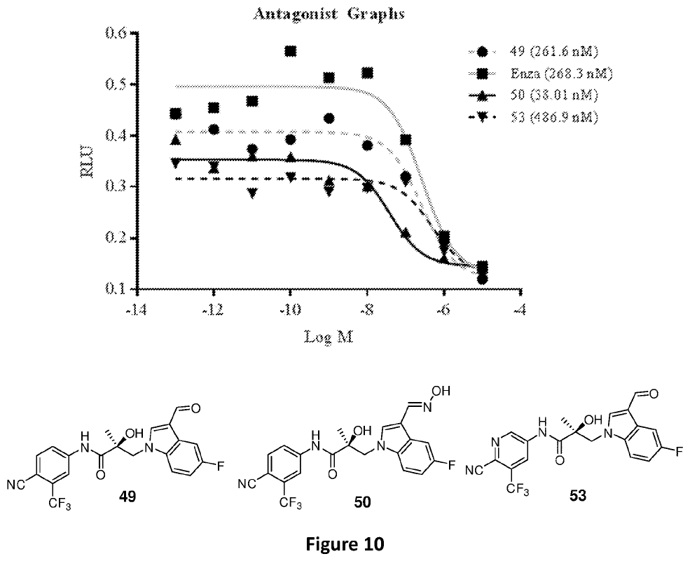

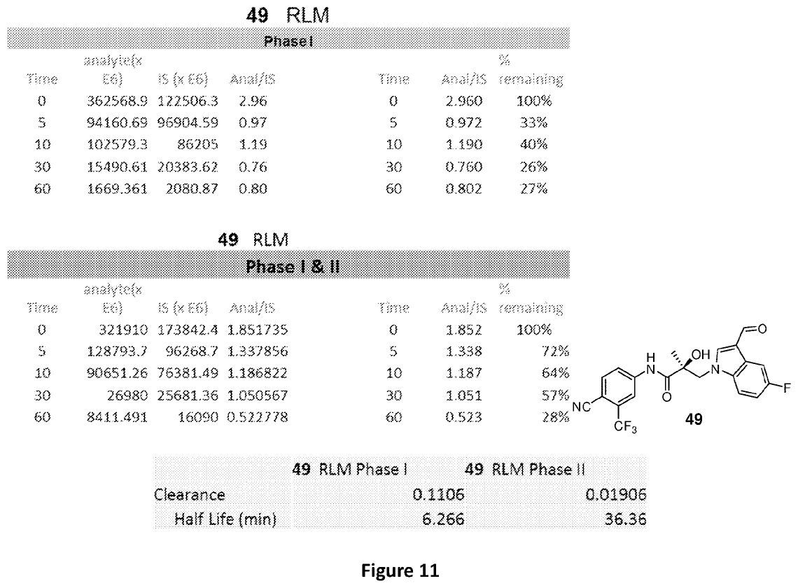

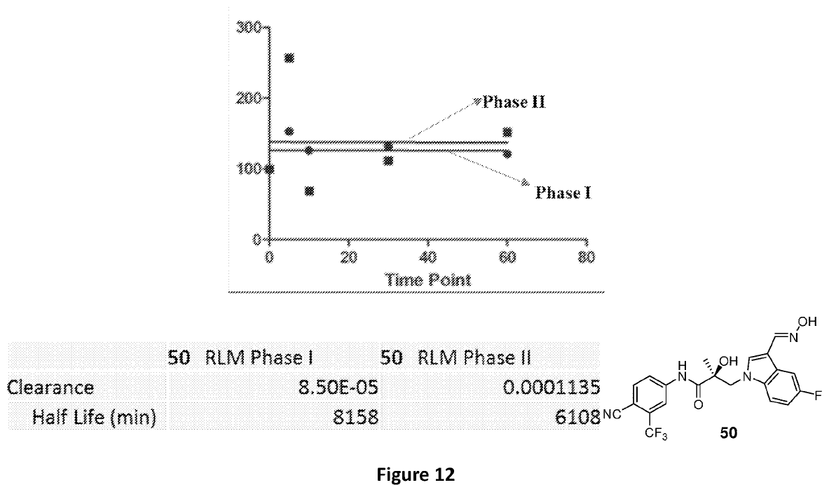

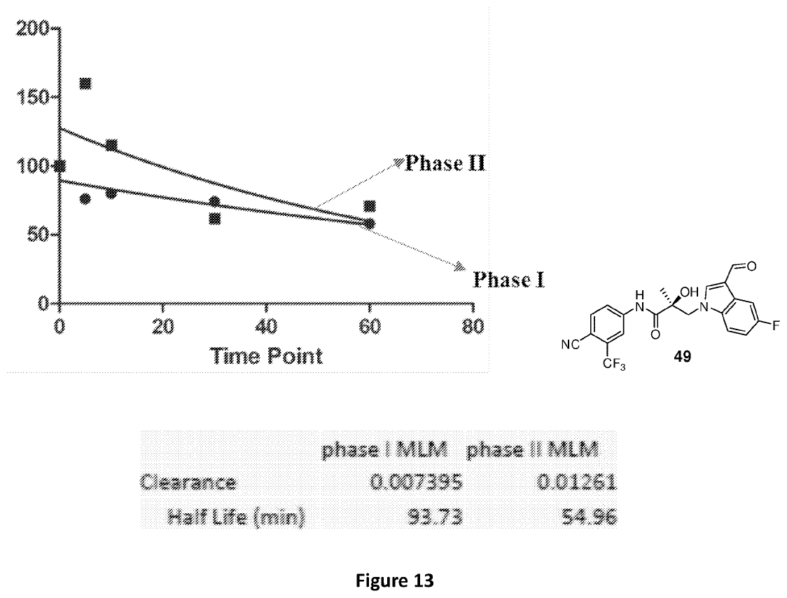

FIGS. 7-10 demonstrate that 56 and 54 (FIGS. 7); 50, 55 and 54 (FIGS. 8 & 9); and 49, 50 and 53 (FIG. 10) exhibited wtAR inhibitory potencies that were comparable (49 and 56) or superior (50, 53-55) to enzalutamide, a known LBD targeted antiandrogen approved for prostate cancer that lacks SARD or AF-1 binding activity. FIGS. 11-13 demonstrate that the 3-formyl (49) or 3-oxime (50) group improves the stability of the indole SARDs of this invention to incubation with MLM or RLM, improving the potential of these compounds to exert an in vivo AR antagonist effect. Half-lives of prior art indoles are found in Table 2 for comparison. These data were collected following the methodology described in Example 2 under the heading “Metabolic stability in Phase I & Phase II pathways” and “LC-MS/MS analysis”.

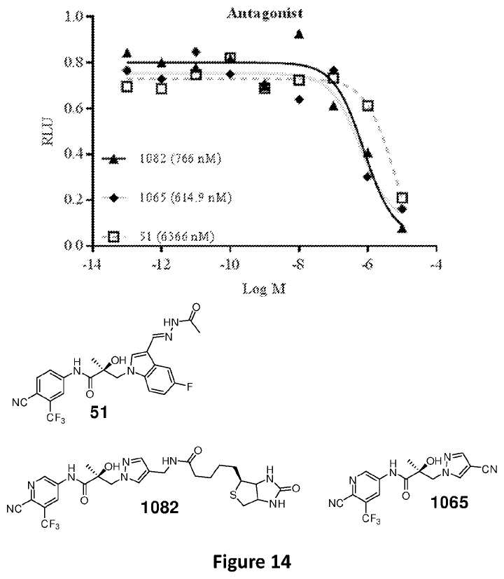

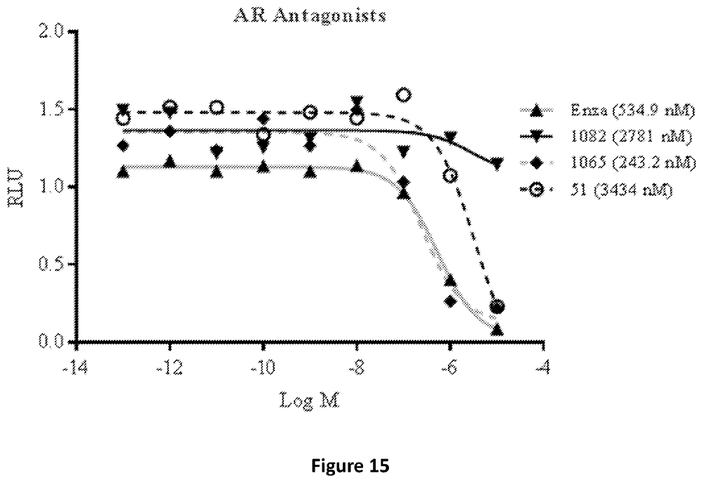

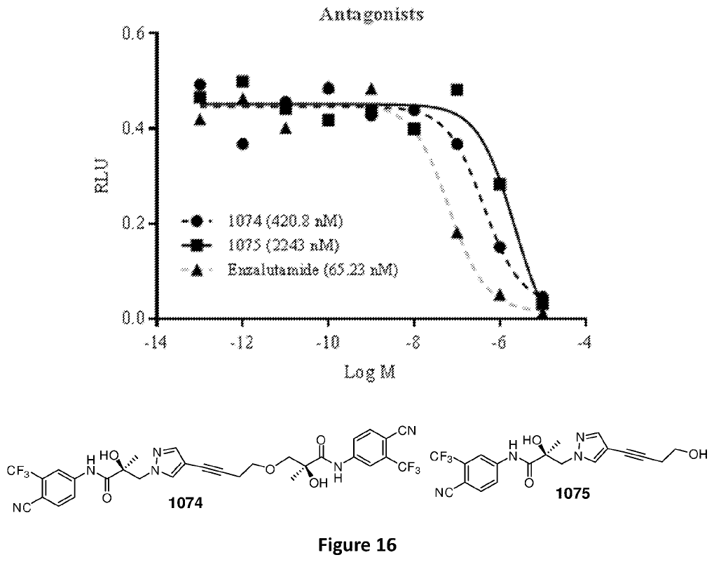

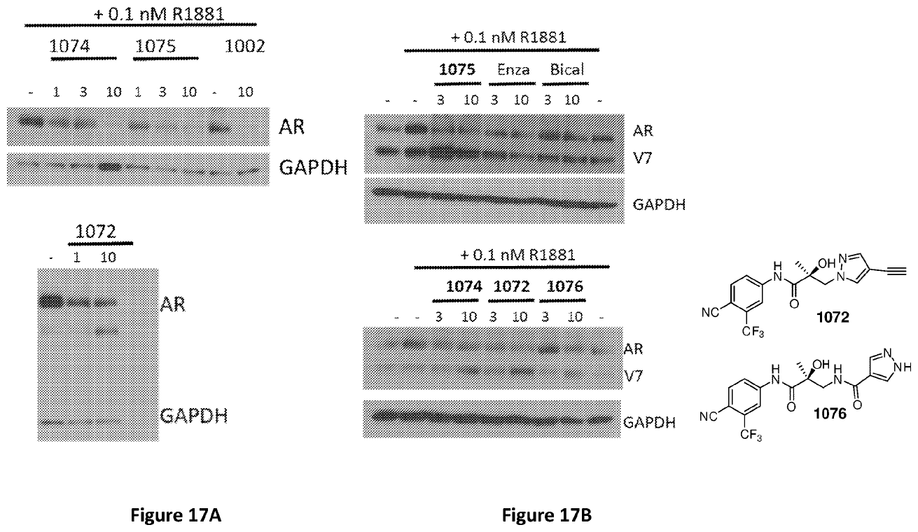

FIGS. 14-16 demonstrate that addition of the 3-acetylhydrazine moiety to the indole (51) produced μM range wtAR inhibitory potency; whereas addition of a biotin side chain (1082) to the 4-position of a pyrazole SARD retained nM to low mM range wtAR inhibitory potency (FIGS. 14 & 15) despite the drastically increased size of the SARD. Similarly, wtAR inhibitory activity was maintained with the 4-position alkyne substituents of a pyrazole SARD. E.g., 1074 also introduces a large 4-position substituent with a second aniline ring and chiral center , whereas 1075 has a smaller alkyne substituent (but-3-yn-1-ol); FIG. 16). This suggests that there is tolerance to steric bulk at the 4-pyrazole position. Further, FIG. 17 demonstrates that 4-alkyne pyrazoles such as 1072 (4-ethinyl), 1074, and 1075 also maintained the ability to degrade AR and AR-V7 (AR SV). 1076 possesses a novel linker element with an extra amide attached to the B -ring pryazole, and also demonstrated some SARD activity despite being an agonist of wtAR (Table 1). FIG. 17 is a degradation experiment as described in Example 2.

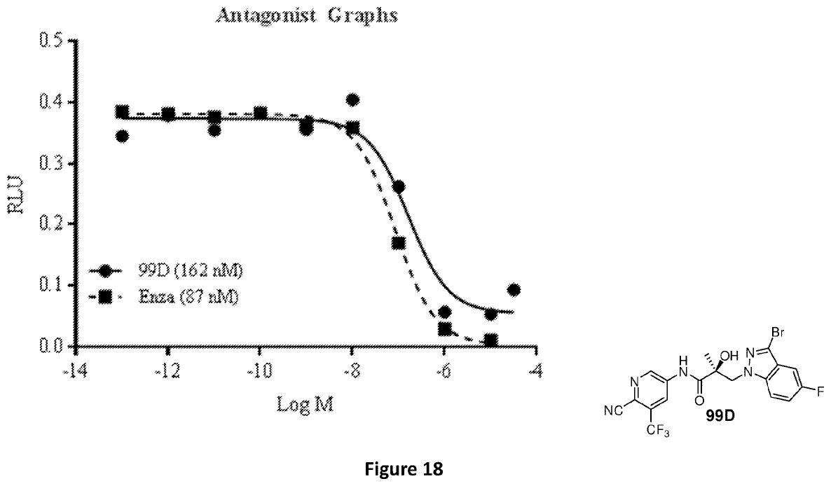

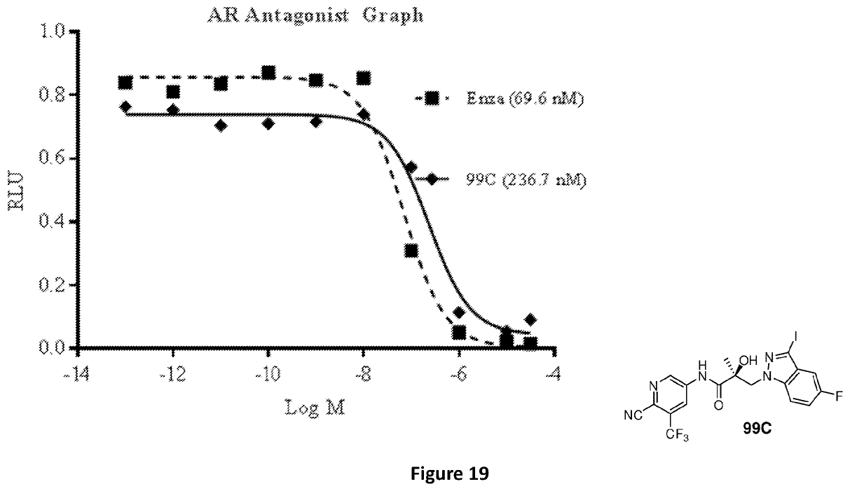

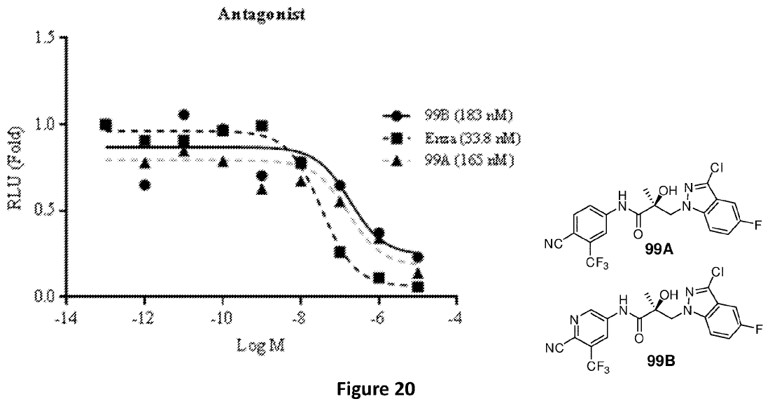

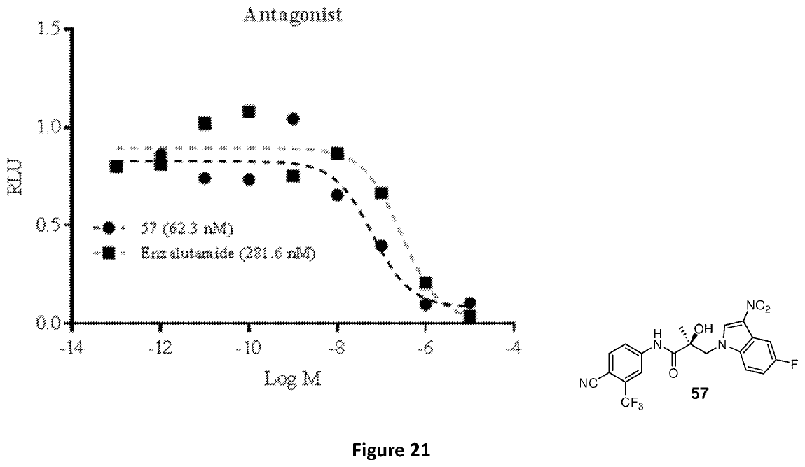

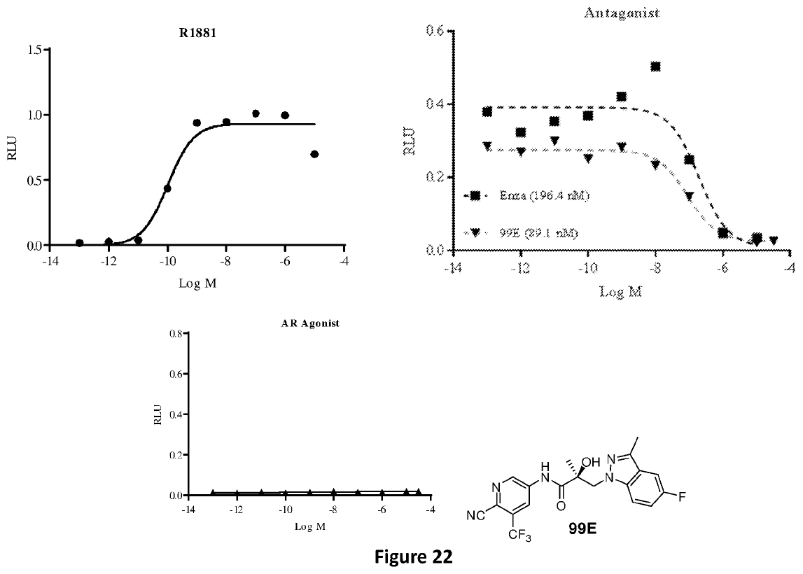

FIGS. 18-22 demonstrate wtAR inhibition for 3-halogen indazoles 99A-99D, 3-methylindazole (99E), and 3-nitro-indole 57. All the indazoles in these figures retained potent nM wtAR inhibition and improved metabolic stability compared to indoles. For example, 3-chloro-5-fluoro indazoles 99A and 99B retained inhibitory potency of 165 nM and 183 nM and the latter was additionally stable in mouse liver microsome (MLM; see Table 1); whereas the 3-chloro-5-fluoro indole 45 was not as potent (367 nM) and had a very short half-life (23.44 min) in MLM (Table 1). In fact, prior to this invention, indoles as a whole have not been stable to in vivo metabolism and correspondingly are limited in their in vivo AR antagonistic effects.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A selective androgen receptor degrader (SARD) compound, or its isomer, optical isomer, or any mixture of optical isomers, pharmaceutically acceptable salt, pharmaceutical product, hydrate or any combination thereof, wherein said SARD compound is represented by a compound of the following structures:

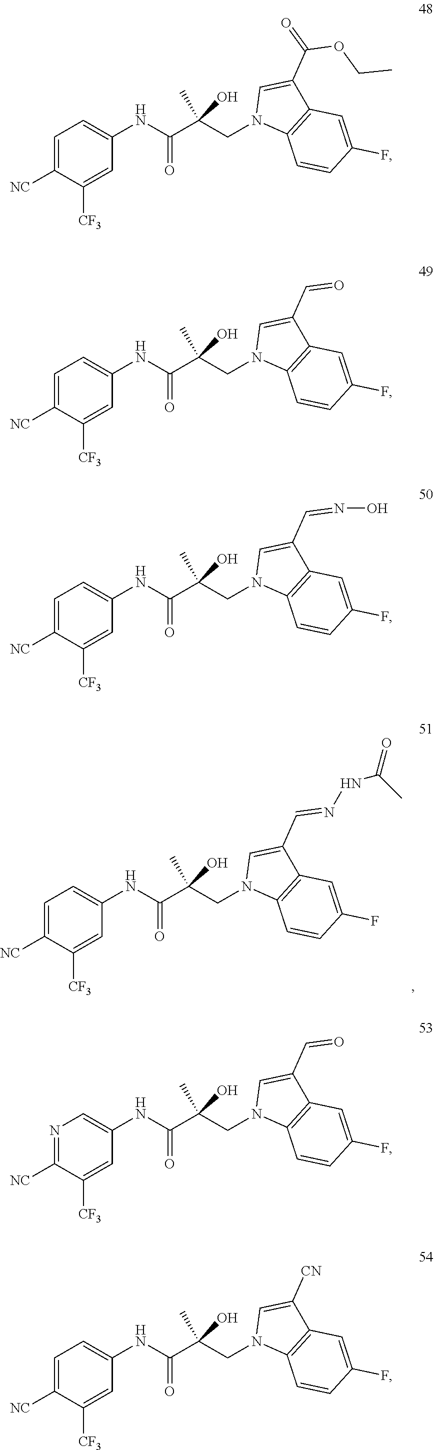 [SMILES: CCOC(=O)c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1ccc(C#N)c(C(F)(F)F)c1 ; C[C@](O)(Cn1cc(/C=N\O)c2cc(F)ccc21)C(=O)Nc1ccc(C#N)c(C(F)(F)F)c1 ; CC(=O)N/N=C/c1cn(C[C@](C)(O)C(=O)Nc2ccc(C#N)c(C(F)(F)F)c2)c2ccc(F)cc12 ; C[C@](O)(Cn1cc(C=O)c2cc(F)ccc21)C(=O)Nc1cnc(C#N)c(C(F)(F)F)c1 ; C[C@](O)(Cn1cc(C#N)c2cc(F)ccc21)C(=O)Nc1ccc(C#N)c(C(F)(F)F)c1]

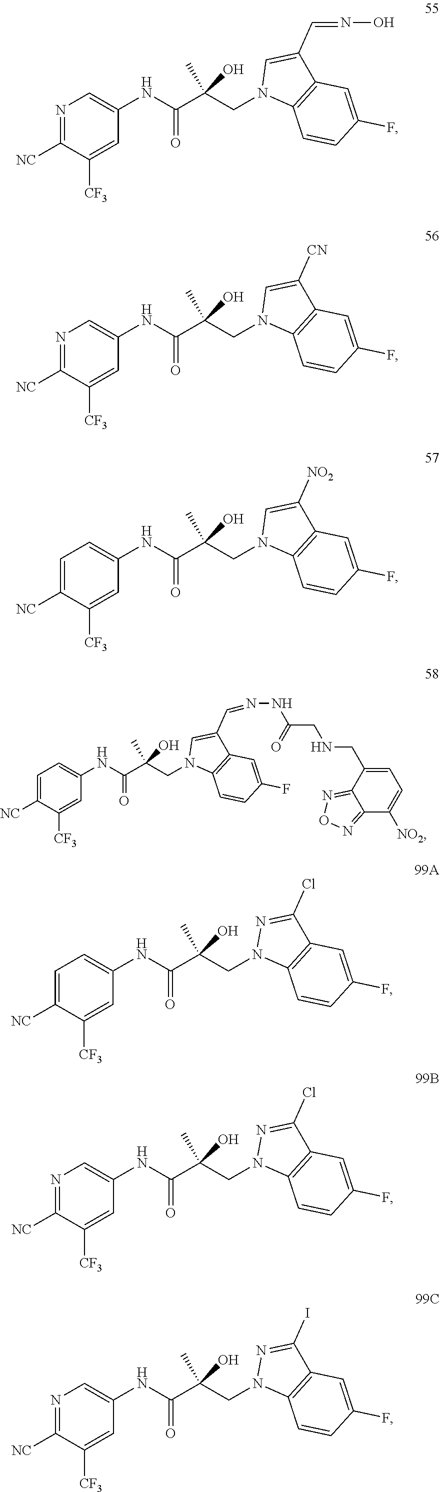

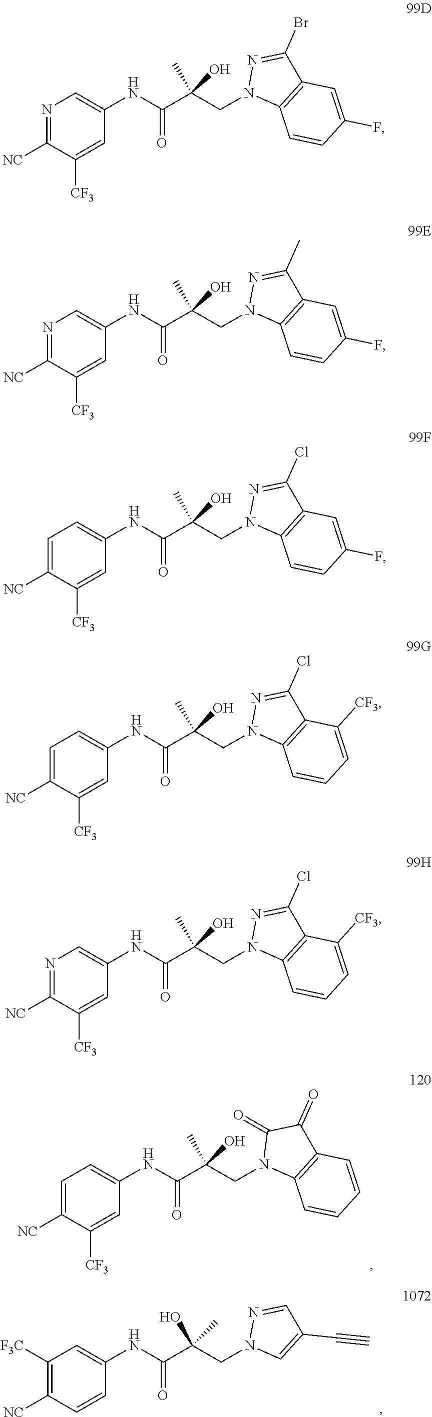

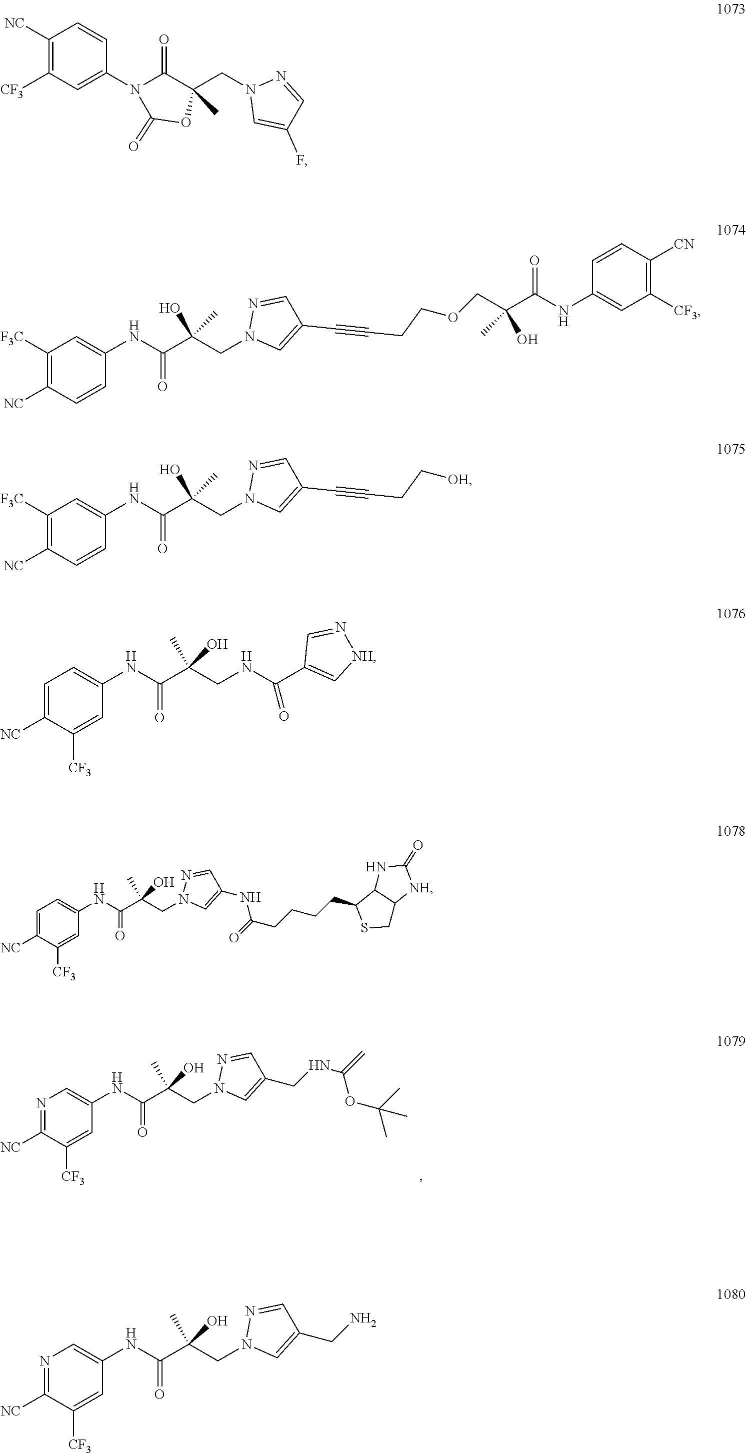

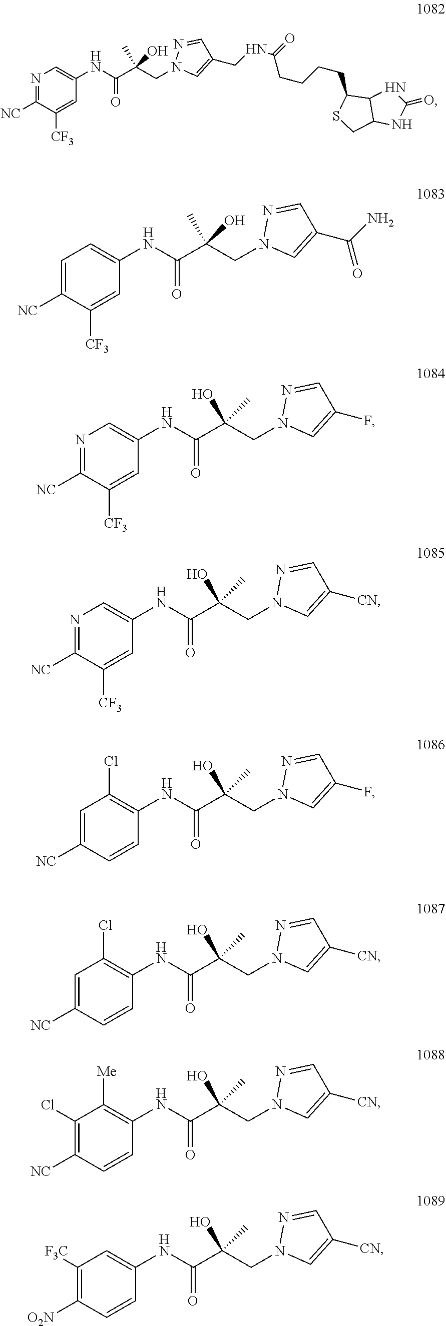

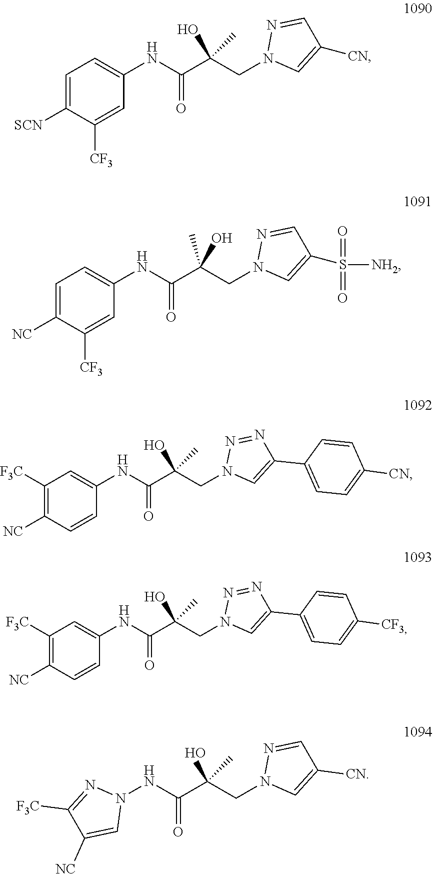

2. The compound according to claim 1, wherein the compound exhibits at least one of binding to the AR through an alternate binding domain in the NTD, AR-splice variant (AR-SV) degradation activity, full length (AR-FL) degradation activity, AR-SV inhibitory activity, AR-FL inhibitory activity, or AR antagonism in vivo of an AR target organ.

3. A pharmaceutical composition comprising a SARD compound according to claim 1, or its isomer, optical isomer, or any mixture of optical isomers, pharmaceutically acceptable salt, pharmaceutical product, hydrate or any combination thereof, and a pharmaceutically acceptable carrier.

4. The pharmaceutical composition according to claim 3, wherein the composition is formulated for topical use.

5. The pharmaceutical composition according to claim 4, wherein the composition is in the form of a solution, lotion, salve, cream, ointment, liposome, spray, gel, foam, roller stick, cleansing soap or bar, emulsion, mousse, aerosol, or shampoo.

6. The pharmaceutical composition according to claim 3, wherein the composition is formulated for oral use.

7. A method of treating an androgen receptor dependent disease or condition in a subject in need thereof, comprising administering to the subject a therapeutically effective amount of a compound of claim 1.

8. The method of claim 7, wherein said androgen receptor dependent disease or condition in said subject responds to at least one of AR-splice variant (AR-SV) degradation activity, full length (AR-FL) degradation activity, AR-SV inhibitory, or AR-FL inhibitory activity.

9. The method of claim 7, wherein said androgen receptor dependent disease or condition is breast cancer in said subject.

10. The method of claim 9, wherein said subject has AR expressing breast cancer, AR-SV expressing breast cancer, and/or AR-V7 expressing breast cancer.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 7, wherein said androgen receptor dependent disease or condition is caused by polyglutamine (polyQ) AR polymorphs in a subject.

32. The method according to claim 31, wherein the polyQ-AR is a short polyQ polymorph or a long polyQ polymorph.

33. (canceled)

34. (canceled)

35. (canceled)

36. A method of treating prostate cancer (PCa) or increasing the survival of a male subject suffering from prostate cancer comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

37. The method according to claim 36, wherein the prostate cancer is at least one of advanced prostate cancer, refractory prostate cancer, castration resistant prostate cancer (CRPC), metastatic CRPC (mCRPC), non-metastatic CRPC (nmCRPC), or high-risk nmCRPC.

38. (canceled)

39. (canceled)

40. (canceled)

41. A method of treating darolutamide resistant prostate cancer in a subject comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

42. A method of treating enzalutamide resistant prostate cancer in a subject comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

43. A method of treating apalutamide resistant prostate cancer in a subject comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

44. A method of treating abiraterone resistant prostate cancer comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

45. A method of treating triple negative breast cancer in a subject comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

46. A method of reducing the levels of AR-splice variants in a subject comprising administering to the subject a therapeutically effective amount of a compound according to claim 1, or its isomer, optical isomer, or any mixture of optical isomers, pharmaceutically acceptable salt, pharmaceutical product, hydrate or any combination thereof.

47. The method according to claim 46, wherein the method further reduces the levels of AR-full length (AR-FL) in the subject.
